SEQUENCE LISTING (108 sequences)

SEQ ID NO: 1 - DNA, 20 bp, Artificial Sequence
  derived from E. coli
ctgctgcatcatgggcgtat

SEQ ID NO: 2 - DNA, 20 bp, Artificial Sequence
  derived from E. coli
ctacatcaatgttaagcggc

SEQ ID NO: 3 - DNA, 20 bp, Artificial Sequence
  derived from E.coli
ggctcaggccgataagaatg

SEQ ID NO: 4 - DNA, 20 bp, Artificial Sequence
  derived from E. coli
agcatcagaacgaccgccag

SEQ ID NO: 5 - DNA, 20 bp, Artificial Sequence
  derived from E. coli
taccaaattgtttctcaatc

SEQ ID NO: 6 - DNA, 20 bp, Artificial Sequence
  derived from E. coli
attgtcgcgcatctccactg

SEQ ID NO: 7 - DNA, 20 bp, Artificial Sequence
  derived from E. coli
ccagtccaaaatgctatgaa

SEQ ID NO: 8 - DNA, 20 bp, Artificial Sequence
  derived from E. coli
gcaagtacgcaaaattgtat

SEQ ID NO: 9 - DNA, 20 bp, Artificial Sequence
  derived from E. coli
gcgtttcgccacttcgccgg

SEQ ID NO: 10 - DNA, 20 bp, Artificial Sequence
  derived from E. coli
agcagtcatgtgttaaattg

SEQ ID NO: 11 - DNA, 20 bp, Artificial Sequence
  derived from E. coli
gagccacatcgaggatgtgt

SEQ ID NO: 12 - DNA, 20 bp, Artificial Sequence
  derived from E. coli
ccgtgatgcggtgcgtcgtc

SEQ ID NO: 13 - DNA, 20 bp, Artificial Sequence
  derived from E. coli
gcaaaagctggataagcagc

SEQ ID NO: 14 - DNA, 20 bp, Artificial Sequence
  derived from E. coli
gttttgttcaatgcgatgca

SEQ ID NO: 15 - DNA, 20 bp, Artificial Sequence
  derived from E. coli
gtgataaaatcgccaacttc

SEQ ID NO: 16 - DNA, 20 bp, Artificial Sequence
  derived from E. coli
ctggcaacgagttaccgata

SEQ ID NO: 17 - DNA, 20 bp, Artificial Sequence
  derived from E. coli
gtatacttgtaattttctta

SEQ ID NO: 18 - DNA, 20 bp, Artificial Sequence
  derived from E. coli
agccctgattaaacatatta

SEQ ID NO: 19 - DNA, 52 bp, Artificial Sequence
  artificial primer
actagtactagtgcaagtacgcaaaattgtatgttttagagctagaaatagc

SEQ ID NO: 20 - DNA, 52 bp, Artificial Sequence
  artificial primer
actagtactagtctgctgcatcatgggcgtatgttttagagctagaaatagc

SEQ ID NO: 21 - DNA, 52 bp, Artificial Sequence
  artificial primer
actagtactagtgcgtttcgccacttcgccgggttttagagctagaaatagc

SEQ ID NO: 22 - DNA, 52 bp, Artificial Sequence
  artificial primer
actagtactagtctacatcaatgttaagcggcgttttagagctagaaatagc

SEQ ID NO: 23 - DNA, 52 bp, Artificial Sequence
  artificial primer
actagtactagtagcagtcatgtgttaaattggttttagagctagaaatagc

SEQ ID NO: 24 - DNA, 52 bp, Artificial Sequence
  artificial primer
actagtactagtggctcaggccgataagaatggttttagagctagaaatagc

SEQ ID NO: 25 - DNA, 52 bp, Artificial Sequence
  artificial primer
actagtactagtgagccacatcgaggatgtgtgttttagagctagaaatagc

SEQ ID NO: 26 - DNA, 52 bp, Artificial Sequence
  artificial primer
actagtactagtagcatcagaacgaccgccaggttttagagctagaaatagc

SEQ ID NO: 27 - DNA, 52 bp, Artificial Sequence
  artificial primer
actagtactagtccgtgatgcggtgcgtcgtcgttttagagctagaaatagc

SEQ ID NO: 28 - DNA, 52 bp, Artificial Sequence
  artificial primer
actagtactagttaccaaattgtttctcaatcgttttagagctagaaatagc

SEQ ID NO: 29 - DNA, 52 bp, Artificial Sequence
  artificial primer
actagtactagtgcaaaagctggataagcagcgttttagagctagaaatagc

SEQ ID NO: 30 - DNA, 52 bp, Artificial Sequence
  artificial primer
actagtactagtattgtcgcgcatctccactggttttagagctagaaatagc

SEQ ID NO: 31 - DNA, 52 bp, Artificial Sequence
  artificial primer
actagtactagtgttttgttcaatgcgatgcagttttagagctagaaatagc

SEQ ID NO: 32 - DNA, 52 bp, Artificial Sequence
  artificial primer
actagtactagtccagtccaaaatgctatgaagttttagagctagaaatagc

SEQ ID NO: 33 - DNA, 50 bp, Artificial Sequence
  artificial primer
agatctagatctaaagaggagaaaggatctatggataagaaatactcaat

SEQ ID NO: 34 - DNA, 32 bp, Artificial Sequence
  artificial primer
ctcgagctcgagttaacgacgaccttcagcaa

SEQ ID NO: 35 - DNA, 33 bp, Artificial Sequence
  artificial primer
gggggggacgtctaagaaaccattattatcatg

SEQ ID NO: 36 - DNA, 32 bp, Artificial Sequence
  artificial primer
gggcccgggcccaagcttcaaaaaaagcaccg

SEQ ID NO: 37 - DNA, 19 bp, Artificial Sequence
  artificial primer
ctattgccagggatggttc

SEQ ID NO: 38 - DNA, 20 bp, Artificial Sequence
  artificial primer
tttcatagaaatgcagcgcc

SEQ ID NO: 39 - DNA, 20 bp, Artificial Sequence
  artificial primer
ggatgttgattctgtcatgg

SEQ ID NO: 40 - DNA, 20 bp, Artificial Sequence
  artificial primer
tatgcattgcagaccttgtg

SEQ ID NO: 41 - DNA, 20 bp, Artificial Sequence
  artificial primer
agtagacgttatcgtcgttg

SEQ ID NO: 42 - DNA, 20 bp, Artificial Sequence
  artificial primer
atggaacgtgagcaggacag

SEQ ID NO: 43 - DNA, 20 bp, Artificial Sequence
  artificial primer
cagccagcgtttcagcatac

SEQ ID NO: 44 - DNA, 20 bp, Artificial Sequence
  artificial primer
tctgctgcgagacataaccc

SEQ ID NO: 45 - DNA, 20 bp, Artificial Sequence
  artificial primer
acttgcaacgaatgttccgc

SEQ ID NO: 46 - DNA, 20 bp, Artificial Sequence
  artificial primer
acgcactaccaccagtaacg

SEQ ID NO: 47 - DNA, 20 bp, Artificial Sequence
  artificial primer
tttgtcttccgggaccagtg

SEQ ID NO: 48 - DNA, 20 bp, Artificial Sequence
  artificial primer
aagcccttcttccagacgtg

SEQ ID NO: 49 - DNA, 20 bp, Artificial Sequence
  artificial primer
aacggcaaagccaaagtgtc

SEQ ID NO: 50 - DNA, 20 bp, Artificial Sequence
  artificial primer
atcgcctggctcatcatacg

SEQ ID NO: 51 - DNA, 20 bp, Artificial Sequence
  artificial primer
gcactggaaatctgtgacgc

SEQ ID NO: 52 - DNA, 20 bp, Artificial Sequence
  artificial primer
ggccagtttgtgattacgcc

SEQ ID NO: 53 - DNA, 20 bp, Artificial Sequence
  artificial primer
ctacgctcccacaaaatgcg

SEQ ID NO: 54 - DNA, 20 bp, Artificial Sequence
  artificial primer
aatcgcgcaaaagctgaagg

SEQ ID NO: 55 - DNA, 20 bp, Artificial Sequence
  artificial primer
gcgattcgccctgcatattg

SEQ ID NO: 56 - DNA, 20 bp, Artificial Sequence
  artificial primer
aacacatgcaagtcgaacgg

SEQ ID NO: 57 - DNA, 20 bp, Artificial Sequence
  artificial primer
aatcccatctgggcacatcc

SEQ ID NO: 58 - DNA, 20 bp, Artificial Sequence
  artificial primer
cgcagtcttgctaccctttg

SEQ ID NO: 59 - DNA, 20 bp, Artificial Sequence
  artificial primer
acgggcaacatcttccacac

SEQ ID NO: 60 - DNA, 20 bp, Artificial Sequence
  artificial primer
gcaagcgtatctctcgtgaa

SEQ ID NO: 61 - DNA, 20 bp, Artificial Sequence
  artificial primer
cgccgaacacacatttatcc

SEQ ID NO: 62 - DNA, 20 bp, Artificial Sequence
  artificial primer
tcggacgcacttttggtcag

SEQ ID NO: 63 - DNA, 20 bp, Artificial Sequence
  artificial primer
caactggaacctttcgggtg

SEQ ID NO: 64 - DNA, 20 bp, Artificial Sequence
  artificial primer
tgatcaaccggttgcggttc

SEQ ID NO: 65 - DNA, 20 bp, Artificial Sequence
  artificial primer
atcttgcagcacttggacgg

SEQ ID NO: 66 - DNA, 20 bp, Artificial Sequence
  artificial primer
aatacgccacccgtgaagac

SEQ ID NO: 67 - DNA, 20 bp, Artificial Sequence
  artificial primer
gacgccccatcatgtaaccg

SEQ ID NO: 68 - DNA, 20 bp, Artificial Sequence
  artificial primer
ggagagcgaattactctggc

SEQ ID NO: 69 - DNA, 20 bp, Artificial Sequence
  artificial primer
ggttagcgaacgtctgaatg

SEQ ID NO: 70 - DNA, 23 bp, Artificial Sequence
  derived from E. coli
ccgatacaattttgcgtacttgc

SEQ ID NO: 71 - DNA, 23 bp, Artificial Sequence
  derived from E. coli
ggctatgttaaaacgcatgaacg

SEQ ID NO: 72 - DNA, 20 bp, Artificial Sequence
  synthetic sequence
atacgcccatgatgcagcag

SEQ ID NO: 73 - DNA, 23 bp, Artificial Sequence
  derived from E. coli
cccatacgcccatgatgcagcag

SEQ ID NO: 74 - DNA, 23 bp, Artificial Sequence
  derived from E. coli
gggtatgcgggtactacgtcgtc

SEQ ID NO: 75 - DNA, 20 bp, Artificial Sequence
  derived from E. coli
gccgcttaacattgatgtag

SEQ ID NO: 76 - DNA, 23 bp, Artificial Sequence
  derived from E. coli
ctacatcaatgttaagcggctgg

SEQ ID NO: 77 - DNA, 23 bp, Artificial Sequence
  synthetic seqeunce
ccagccgcttaacattgatgtag

SEQ ID NO: 78 - DNA, 23 bp, Artificial Sequence
  derived from E. coli
ccccattcttatcggcctgagcc

SEQ ID NO: 79 - DNA, 23 bp, Artificial Sequence
  derived from E. coli
ggctcaggccgataagaatgggg

SEQ ID NO: 80 - DNA, 23 bp, Artificial Sequence
  derived from E. coli
agcatcagaacgaccgccagagg

SEQ ID NO: 81 - DNA, 23 bp, Artificial Sequence
  derived from E. coli
cctctggcggtcgttctgatgct

SEQ ID NO: 82 - DNA, 23 bp, Artificial Sequence
  derived from E. coli
taccaaattgtttctcaatctgg

SEQ ID NO: 83 - DNA, 23 bp, Artificial Sequence
  derived from E. coli
ccagattgagaaacaatttggat

SEQ ID NO: 84 - DNA, 23 bp, Artificial Sequence
  derived from E. coli
ccgcagtggagatgcgcgacaat

SEQ ID NO: 85 - DNA, 23 bp, Artificial Sequence
  derived from E. coli
attgtcgcgcatctccactgcgg

SEQ ID NO: 86 - DNA, 23 bp, Artificial Sequence
  derived from E. coli
ccattcatagcattttggactgg

SEQ ID NO: 87 - DNA, 23 bp, Artificial Sequence
  derived from E. coli
ccagtccaaaatgctatgaatgg

SEQ ID NO: 88 - DNA, 23 bp, Artificial Sequence
  derived from E. coli
cccccggcgaagtggcgaaacgc

SEQ ID NO: 89 - DNA, 23 bp, Artificial Sequence
  derived from E. coli
gcgtttcgccacttcgccggggg

SEQ ID NO: 90 - DNA, 20 bp, Artificial Sequence
  synthetic sequence
ccggcgaagtggcgaaacgc

SEQ ID NO: 91 - DNA, 23 bp, Artificial Sequence
  derived from E. coli
agcagtcatgtgttaaattgagg

SEQ ID NO: 92 - DNA, 23 bp, Artificial Sequence
  derived from E. coli
cctcaatttaacacatgactgct

SEQ ID NO: 93 - DNA, 23 bp, Artificial Sequence
  derived from E. coli
ccaacacatcctcgatgtggctc

SEQ ID NO: 94 - DNA, 23 bp, Artificial Sequence
  derived from E. coli
gagccacatcgaggatgtgttgg

SEQ ID NO: 95 - DNA, 23 bp, Artificial Sequence
  derived from E. coli
cctgacgacgcaccgcatcacgg

SEQ ID NO: 96 - DNA, 23 bp, Artificial Sequence
  derived from E. coli
ccgtgatgcggtgcgtcgtcagg

SEQ ID NO: 97 - DNA, 23 bp, Artificial Sequence
  derived from E. coli
gcaaaagctggataagcagcagg

SEQ ID NO: 98 - DNA, 23 bp, Artificial Sequence
  derived from E. coli
cctgctgcttatccagcttttgc

SEQ ID NO: 99 - DNA, 23 bp, Artificial Sequence
  derived from E. coli
ccttgcatcgcattgaacaaaac

SEQ ID NO: 100 - DNA, 23 bp, Artificial Sequence
  derived from E. coli
gttttgttcaatgcgatgcaagg

SEQ ID NO: 101 - DNA, 164 bp, Artificial Sequence
  partial plasmid vector sequence
agatctttgacagctagctcagtcctaggtataatactagtaactttcagtttagcggtctgttttagagctagaaatagcaagttaaaataaggctagtccgttatcaacttgaaaaagtggcaccgagtcggtgctttttttgaagcttgggcccgaacaaa

SEQ ID NO: 102 - DNA, 164 bp, Artificial Sequence
  partial plasmid vector sequence
tttgttcgggcccaagcttcaaaaaaagcaccgactcggtgccactttttcaagttgataacggactagccttattttaacttgctatttctagctctaaaacagaccgctaaactgaaagttactagtattatacctaggactgagctagctgtcaaagatct

SEQ ID NO: 103 - DNA, 51 bp, Artificial Sequence
  artificial primer
actagtactagtgcaaaagctggataagcagcgttttagagctagaaatag

SEQ ID NO: 104 - DNA, 32 bp, Artificial Sequence; identical to SEQ ID NO: 36
  artificial primer

SEQ ID NO: 105 - DNA, 13 bp, Artificial Sequence
  synthetic sequence
  N_region (1)..(4): n = any nucleic acid
  misc_feature (1)..(4): n is a, c, g, or t
  N_region (10)..(12): n = any nucleic acid
  misc_feature (11)..(13): n is a, c, g, or t
nnnntctagannn

SEQ ID NO: 106 - DNA, 12 bp, Artificial Sequence
  synthetic sequence
  N_region (1)..(3): n = any nucleic acid
  misc_feature (1)..(3): n is a, c, g, or t
  N_region (10)..(12): n = any nucleic acid
  misc_feature (10)..(12): n is a, c, g, or t
nnntctagannn

SEQ ID NO: 107 - DNA, 12 bp, Artificial Sequence
  synthetic sequence
  N_region (1)..(3): n = any nucleic acid
  misc_feature (1)..(3): n is a, c, g, or t
  N_region (10)..(12): n = any nucleic acid
  misc_feature (10)..(12): n is a, c, g, or t
nnncctaggnnn

SEQ ID NO: 108 - DNA, 12 bp, Artificial Sequence
  synthetic sequence
  N_region (1)..(3): n = any nucleic acid
  misc_feature (1)..(3): n is a, c, g, or t
  N_region (10)..(12): n = any nucleic acid
  misc_feature (10)..(12): n is a, c, g, or t
nnncctaggnnn

(19) United States
(12) Patent Application Publication
(10) Pub. No.: US 2020/0385715 A1
(43) Pub. Date: Dec. 10, 2020

(54) COMPOSITIONS AND METHODS FOR ALTERING BACTERIA FITNESS

(71) Applicant: THE REGENTS OF THE UNIVERSITY OF COLORADO, A BODY CORPORATE, Denver, CO (US)

(72) Inventors: Anushree Chatterjee, Lafayette, CO (US); Peter Otoupal, Boulder, CO (US)

(73) Assignee: THE REGENTS OF THE UNIVERSITY OF COLORADO, A BODY CORPORATE, Denver, CO (US)

(21) Appl. No.: 16/300,372

(22) PCT Filed: May 11, 2017
(86) PCT No.: PCT/US2017/032298

§ 371 Date: Nov. 9, 2018

Related U.S. Application Data
(60) Provisional application No. 62/334,967, filed on May 11, 2016.

(51) Int. Cl. C12N 15/113; C07K 14/00; C12N 1/36; C12N 15/10; C12N 15/63; C12N 9/22

(52) U.S. Cl. CPC C12N 15/113; C07K 14/003; C12N 1/36; C12N 2310/20; C12N 15/63; C12N 9/22; C12N 15/102

(57) ABSTRACT

Embodiment disclosed herein concern compositions and methods for altering bacterial fitness. In certain aspects, altering bacterial fitness slows or prevents development of adaptive resistance to antibiotics in bacteria. In certain embodiments disclosed herein, bacterial fitness is altered by perturbing expression of a group of target genes.

### CROSS-REFERENCE TO RELATED APPLICATIONS

This application claims priority to U.S. Provisional Application No. 62/334,967, filed on May 11, 2016, the entire disclosure of which is expressly incorporated herein by reference for all purposes.

### STATEMENT REGARDING FEDERALLY SPONSORED RESEARCH

This invention was made with government support under grant number CMMI1235532 awarded by the National Science Foundation, and grant number DGE1144083, awarded by the National Science Foundation. The government has certain rights in the invention.

### SEQUENCE LISTING

The instant application contains a Sequence Listing which has been submitted via EFS-web and is hereby incorporated by reference in its entirety. The ASCII copy, created on May 11, 2017, is named SL_466888.54_ST25.txt and is 22545 bytes in size.

### FIELD

Embodiments herein provide methods, compositions, and uses for altering bacterial fitness. Certain embodiments concern slowing or preventing bacteria from developing adaptive resistance to antibiotics by altering bacterial fitness. In certain embodiments, these effects can be through controlled negative epigenetic epistasis. In some embodiments, development of adaptive resistance to antibiotics can be inhibited in a subject undergoing an antibiotic therapy. Other embodiments are directed to CRISPR/Cas systems or artificial nucleic acid-based systems for altering bacterial fitness through controlled negative epigenetic epistasis. In some embodiments, the CRISPR/Cas system or PNA-based system modifies expression levels of one or more bacterial stress response genes, bacterial conserved genes, and/or bacterial essential genes.

### BACKGROUND

Antibiotic resistance is one of the world's most pressing health problems. Drug-resistant bacteria infect more than 2 million Americans every year, and are responsible for 23,000 deaths annually in the United States alone. Increasing rates of antibiotic-resistant bacterial infections observed in clinical settings is a result of the misuse and overuse of antibiotics prescribed in veterinary and human medicine. This high volume is largely due to inappropriate prescribing of antibiotics. This is problematic, as use of antibiotics can increase selective pressure in a population of bacteria, resulting in the survival of drug resistant bacteria. These selective pressures and a resulting drug resistant bacterium can result from a single regimen of antibiotics.

While antibiotic resistance continues to be a major global public health concern, antibiotic development continues to stagnate; drug screens for new antibiotics tend to rediscover the same lead compounds, antibiotics represent a relatively poor return on research and development investment compared to other classes of drugs, and antibiotic approval through the U.S. FDA has become confusing and generally infeasible over the past decade. The number of new drugs to replace antibiotics rendered ineffective due to the emergence of antibiotic resistant bacteria is not adequate to meet demand.

### SUMMARY

The present disclosure provides compositions and methods for altering bacterial fitness. In certain aspects, altering bacterial fitness slows or prevents development of adaptive resistance to antibiotics in bacteria. In certain embodiments disclosed herein, bacterial fitness is altered by perturbing expression of a group of target genes.

In some embodiments, methods for altering bacterial fitness of a bacterium comprise modulating gene expression of at least 3 genes in the bacterium. In some embodiments, the method kills the bacterium. In other embodiments, the method slows or prevents development of adaptive resistance to antibiotics in the bacterium. In some embodiments, the at least 3 genes comprise bacterial stress response genes, bacterial essential genes, or a combination of bacterial stress response genes and bacterial essential genes.

In some embodiments, stress response genes comprise mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ifs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsD, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and the bacterial essential genes comprise dfp, topA, zwf, and frr. In other embodiments, the at least 3 genes are selected from the group of mutS, soxS, tolC, recA, dfp, zwf, topA, frr, and rfp.

In some embodiments, the at least 3 genes comprise a group of genes selected from the group of mutS, soxS, and to/C; mutS, soxS, and recA; mutS, tolC, and recA; soxS, to/C, and recA; dfp, zwf, and topA; dfp, zwf, and frr; dfp, topA, and frr; mutS, soxS, tolC, and recA; dfp, zwf, topA, and frr; and mutS, soxS, topA, and frr.

In some embodiments, modulating the gene expression of the at least 3 genes comprises modulating the gene expression at a transcriptional level, at a post-transcriptional level, or at both a transcriptional and a post-transcriptional level. In some embodiments, modulating the gene expression comprises modulating the gene expression at a transcriptional level by delivering to the bacterium at least one of a CRISPR/Cas system, a transcription activator-like effector (TALE) system, a zinc-finger protein system, a synthetic polyamide system, and a meganuclease system. In other embodiments, modulating the gene expression comprises modulating the gene expression at a post-transcriptional level by delivering to the bacterium at least one of a morpholino-based system, a peptide nucleic acid-based system, and a locked nucleic acid-based system.

In some embodiments, modulating the gene expression of the at least 3 genes comprises delivering a CRISPR/Cas system to the bacterium. In some embodiments, the CRISPR/Cas system comprises a catalytically dead CRISPR-associated (dCas) protein and at least three guide RNA (gRNA) molecules, wherein each of the at least three gRNA molecules comprise a CRISPR-associated (Cas) protein binding site and a targeting RNA sequence. In some embodiments, the targeting RNA sequence of each of the at least three gRNA molecules targets one gene of the at least 3 genes. In some embodiments, the targeting RNA sequence comprises a nucleic acid sequence that is complementary to a nucleic acid sequence of the one gene. In some embodiments, the nucleic acid sequence of the one gene comprises a regulatory region of the one gene.

In some embodiments, the CRISPR/Cas system further comprises a transcriptional effector molecule associated with the dCas protein. In some embodiments, the transcriptional effector molecule is selected from the group of a DNA methylase, a histone acetylase, and an RNA polymerase ω-subunit.

In some embodiments, components of the CRISPR/Cas system are delivered to the bacterium as naked components, or as encapsulated components. In some embodiments, encapsulated components are encapsulated in one or more nanoparticles. In some embodiments, a surface of the one or more nanoparticles comprises at least one cell-specific targeting ligand for the bacterium selected from the group of an antibody, a cell-penetrating peptide, or a combination thereof.

In some embodiments, the dCas protein is encoded by a first nucleic acid sequence and each of the at least three gRNA molecules is encode by an additional nucleic acid sequence, and wherein at least one expression vector comprises the first nucleic acid sequence and the additional nucleic acid sequences.

In some embodiments, the dCas protein and the transcriptional effector molecule are encoded by a first nucleic acid sequence and each of the at least three gRNA molecule is encoded by an additional nucleic acid sequence, and wherein at least one expression vector comprises the first nucleic acid sequence and the additional nucleic acid sequences.

In some embodiments, a single expression vector comprises the first nucleic acid sequence and the additional nucleic acid sequences. In other embodients, two or more expression vectors each comprise one of, or a combination of, the first nucleic acid sequence and one or more of the additional nucleic acid sequences.

In some embodiments, the at least one expression vector is delivered to the bacterium by a bacteriophage, a donor cell, or as one or more encapsulated expression vectors.

In some embodiments, modulating the gene expression of the at least 3 genes comprises delivering at least three peptide nucleic acids to the bacterium, wherein each of the at least three peptide nucleic acids comprises a sequence of 5 to 20 nucleic acids capable of hybridizing to a target sequence of one of the at least three genes.

In some embodiments, wherein the at least three peptide nucleic acids are encapsulated in one or more nanoparticles.

In some embodiments, a method provided herein is carried out in vivo.

In other embodiments, a method provided herein is carried out in vitro.

Certain embodiments described herein provide a CRISPR/Cas system for altering bacterial fitness of a bacterium comprising: a catalytically-dead CRISPR-associated (dCas) protein; and at least three guide RNA (gRNA) molecules, wherein each of the at least three gRNA molecules comprise a CRISPR-associated (Cas) protein binding site and a targeting RNA sequence specific for a unique gene of the bacterium. In some embodiments, the at least three gRNA molecules target stress response genes, bacterial essential genes, or a combination of bacterial stress response genes and bacterial essential genes.

Other embodiments described herein provide a CRISPR/Cas system for altering bacterial fitness of a bacterium, comprising at least one expression vector, the at least one expression vector comprising: a first nucleic acid sequence encoding a catalytically-dead CRISPR-associated (dCas) protein; and at least three additional nucleic acid sequences, wherein each of the at least three additional nucleic acid sequences encodes a unique guide RNA (gRNA) molecule, wherein each unique gRNA molecule comprise a CRISPR-associated (Cas) protein binding site and a targeting RNA sequence specific for a unique gene of the bacterium. In some embodiments, the at least three additional nucleic acid sequences each encode a unique gRNA that targets a stress response gene or a bacterial essential gene.

Some embodiments described herein provide a peptide nucleic acid system for altering bacterial fitness of a bacterium, comprising at least three peptide nucleic acids, wherein each of the at least three peptide nucleic acids comprises a sequence of 5 to 20 nucleic acids capable of hybridizing to a target sequence of a unique gene of the bacterium. In some embodiments, the at least three peptide nucleic acids each target a stress response gene or a bacterial essential gene.

Other embodiments described herein provide a pharmaceutical composition comprising a CRISPR/Cas system, a vector-expressible CRISPR/Cas system, or a peptide nucleic acid system described herein, or a combination thereof, and a pharmaceutically acceptable vehicle.

In some embodiments, the pharmaceutical composition of claim 57, further comprising at least one antibiotic. In some embodiments, the pharmaceutical composition of claim 58, wherein the at least one antibiotic is selected from the group of penicillins, cephalosporins, carbacephems, cephamycins, carbapenems, monobactams, aminoglycosides, glycopeptides, quinolones, tetracyclines, macrolides, and fluoroquinolones.

In some embodiments, the pharmaceutical composition can be used in a method of killing bacteria in a subject, or slowing or preventing development of adaptive resistance to antibiotics in bacteria in a subject, the method comprising administering an effective amount of the pharmaceutical composition to the subject. In some embodiments, at least one antibiotic to the subject prior to, concurrently with, or both prior to and concurrently with the pharmaceutical composition of claim 57.

### BRIEF DESCRIPTION OF THE DRAWINGS

The following drawings form part of the instant specification and are included to further demonstrate certain aspects of particular embodiments herein. The embodiments may be better understood by reference to one or more of these drawings in combination with the detailed description presented herein.

FIGS. 1A-1D represent examples of design and characterizations of synthetic CRISPR constructs perturbing gene expression according to some embodiments described herein.

FIGS. 2A-2D represent altered growth characteristics induced by modulating gene expression during stress exposure according to some embodiments described herein.

FIGS. 3A-3F represent competition assay results, illustrating changes in fitness resulting from modulation of gene expression according to some embodiments described herein.

FIGS. 4A-4E represent a schematic of experimental design to investigate reversibility of phenotypic changes observed during growth under stress (FIG. 4A), and experimental results (FIGS. 4B-4E) according to some embodiments described herein.

FIGS. 5A-5F illustrate schematic representations of utilization of CRISPR constructs to simultaneously modulate expression of multiple genes according to some embodiments described herein.

FIGS. 6A-6B represent induction of negative epistasis by simultaneous modulation of gene expression according to some embodiments described herein.

FIGS. 7A-7B are schematics representing inhibition target sequences (FIG. 7A) and activation target sequences (FIB. 7B) for the bacterial stress response genes soxS, tolC, acrA, recA, dinB and marA according to some embodiments described herein.

FIG. 8 is a plot illustrating impacts of CRISPR/Cas gene expression modulation constructs as relative fold changes in gene expression from the wild type for genes upstream (nudF, ygbA, lafU) and downstream (ygiB, pphB, yafN) of the intended CRISPR modulation targets to/C, mutS and dinB according to some embodiments described herein.

FIG. 9 represents growth rates of MG1655 strains carrying sgRNA and dCas9 (or dCas9-ω) constructs according to some embodiments described herein.

FIG. 10 is a schematic representing one experimental design for determination of minimum inhibitory concentrations (MIC) according to some embodiments described herein.

FIG. 11 is a graphical representation of the linear fit of normalized lag time (τ-1) and growth rate (μ) from all 14 single-gene modulation strains grown under the five stress conditions according to some embodiments described herein.

FIG. 12 represents 3 replications of an FACS competition assay with MG1655-acrAi and MG1655-mCherry according to some embodiments described herein.

FIGS. 13A-13B are schematic representations of the experimental design for constructing single (FIG. 13A) and multi-target (FIG. 13B) plasmids according to some embodiments described herein.

FIG. 14 is a histogram plot illustrating epistatic interactions on normalized growth rate (μₙₒᵣₘ) of multiple-gene targeting strains according to some embodiments described herein.

FIG. 15 is a histogram plot illustrating epistatic interactions on normalized lag time (norm) of multiple-gene targeting strains according to some embodiments described herein.

FIG. 16 is a box plot illustrating the distribution of estimated epistatic impacts on normalized lag time (τ⁻¹ₙₒᵣₘ) and growth rate (μₙₒᵣₘ), clustered by stress according to some embodiments described herein.

FIG. 17 is a schematic depicting stratagem for assembling multiple-targeting sgRNA plasmids according to some embodiments described herein.

FIG. 18 represents all known genetic interactions to date of gene targets investigated in Example 8.

FIG. 19 represents a bar graph indicating qPCR results of CRIPSR perturbation on gene expression, as quantified by changes in mRNA concentration in relation to the control 1-target RFPi strain according to some embodiments described herein.

FIG. 20 is a schematic depicting experimental procedure for determining fitness values according to some embodiments described herein.

FIG. 21 represents fitness impacts of gene expression perturbations according to some embodiments described herein.

FIG. 22 represents growth curves of control strains under various concentrations of aTc according to some embodiments described herein.

FIG. 23 represents growth curves of select strains under various levels of induction of the CRISPR perturbation system of Example 8 according to some embodiments described herein.

FIG. 24 represents normalized growth rates of strains under different levels of induction of the CRISPR perturbation system of Example 8 according to some embodiments described herein.

FIGS. 25A-25B represent epistasis resulting from two or more gene perturbations according to some embodiments described herein.

FIG. 26 represents average absolute Pearson Correlation Coefficient (PCC) of all sets of statistically significant genetic interactions determined from a previous study according to some embodiments described herein.

FIG. 27 represents the change in ciprofloxacin MIC on adapting populations over three days of continuous exposure according to some embodiments described herein.

FIG. 28 represents average MICs of strain dzTf compared to the control in a separate experiment to validate the results of FIG. 27 according to some embodiments described herein.

FIG. 29 is a histogram plot illustrating mutation rates of strains as determined by a mutation fluctuation assay according to some embodiments described herein.

### DEFINITIONS

“Pharmaceutically acceptable” refers to approved or approvable by a regulatory agency of a government, such as the US FDA or the EMA, or listed in the U.S. Pharmacopoeia or other generally recognized pharmacopoeia for use in mammals and/or animals, and more particularly in humans.

“Pharmaceutically acceptable vehicle” can mean to a pharmaceutically acceptable diluent, a pharmaceutically acceptable adjuvant, a pharmaceutically acceptable excipient, a pharmaceutically acceptable carrier, or a combination of any of the foregoing with which one or more opioid antagonists disclosed by the present disclosure may be administered to a subject, which does not destroy the pharmacological activity thereof and which is non-toxic when administered in doses sufficient to provide a therapeutically effective amount of the opioid antagonist(s).

“Pharmaceutical composition” can include, for example, a therapeutically active CRISPR/Cas system, vector-expressible CRISPR/Cas system, artificial nucleic acid-based system (e.g., PNA system), or other gene regulatory system described herein, and at least one pharmaceutically acceptable vehicle, with which a gene regulatory system can be administered to a subject.

“Subject” refers to a human, domesticated animal such as a dog, cat or horse, or food animal, such as cattle, sheep and goats, pigs, poultry, honey bees, and fish.

“Therapeutically effective amount” refers to number of copies of a gene regulatory system described herein that, when administered to a subject, is sufficient to slow or halt development of antibiotic resistance in bacteria or kill bacteria. The “therapeutically effective amount” may vary depending, for example, on efficient delivery of the gene regulatory system to a target cell, specificity to target sequences, half-life, route of administration, the age, weight, and/or health of the subject to be treated, and the judgment of the prescribing physician. An appropriate amount in any given instance may be ascertained by those skilled in the art or capable of determination by routine experimentation.

“Therapeutically effective dose” refers to a dose that provides effective slowing or halting of development of antibiotic resistance in bacteria or killing of bacteria. A therapeutically effective dose may vary from compound to compound, and from subject to subject, and may depend upon factors such as the condition of the subject and the route of delivery. A therapeutically effective dose may be determined in accordance with routine procedures known to those skilled in the art.

### DETAILED DESCRIPTION

In the following sections, various representative methods and compositions are described in order to detail various embodiments. It will be obvious to one skilled in the art that practicing the various embodiments does not require the employment of all or even some of the specific details outlined herein, but rather that vector backbones, cell-targeting antibodies, cell-penetrating peptides and other specific details may be modified through routine experimentation. In some embodiments, well-known methods or components have not been included in the description.

Methods for identifying genes and combinations of genes resulting in reduced bacterial fitness and/or negative epigenetic epistasis.

Embodiments of the present disclosure provide methods for identifying individual combinations of bacterial genes, that when perturbed, result in reduced bacterial fitness, and in a group of genes, negative epigenetic epistasis. In some embodiments, reducing bacterial fitness can slow or prevent the development of antibiotic resistance in bacteria, or can kill the bacteria. Other embodiments provide novel methods, compositions, and methods of use for altering bacterial fitness of a bacterium. In some embodiments, the methods, compositions, and methods of use can be used to slow or prevent development of adaptive resistance to antibiotics in a bacterium. In some embodiments, the methods, compositions and methods of use can be used to kill a bacterium or prevent it from reproducing (i.e, as an antibiotic).

In some embodiments, methods are provided for identifying combinations of bacterial genes that when perturbed (i.e., expression is modified), result in negative epigenetic epistasis and a reduction in bacterial fitness. In certain embodiments, altering bacterial fitness can reduce or prevent development of adaptive resistance of bacteria to antibiotics. As described herein, relatively small manipulations in expression patterns of bacterial genes, such as stress-response genes and conserved genes, can be sufficient to alter bacterial fitness and growth characteristics during the early stages of stress exposure.

In some embodiments, the individual genes and combinations of bacterial genes can be identified by screening combinations of genes using one or more biological tools capable of inhibiting or activating expression of the genes in a single bacterium, resulting in a bacterial strain with a perturbed transcriptome. In some embodiments, modulation of the expression of a single gene is tested to determine whether the modulation of expression of the gene affects bacterial fitness. In some embodiments, modulation (e.g., increasing or decreasing) of expression of the single gene can slow or prevent the development of antibiotic resistance in a bacterium. In some embodiments, modulation (e.g., increasing or decreasing) of the expression of two or more genes is tested to determine whether the modulation of expression of the two or more genes results in negative epigenetic epistasis, as determined by the difference between a measured bacterial fitness (ω) of the perturbed strain and a calculated expected fitness (ω_{E}) for the perturbed strain. In some embodiments, the expression of at least 3 genes or of at least 4 genes is modulated.

In some embodiments, fitness for each perturbed strain can be calculated by competing the perturbed strain against a non-perturbed control strain. In some embodiments, the control strain can be a wild-type strain with no gene expression modulation. In other embodiments, the control strain can include a non-functional version of the biological tool used to modulate the expression of the two or more genes in the perturbed strain. Using a competition growth assay, colony counts of the perturbed strain and control strain can be used to determine bacterial fitness. In some embodiments, the competition growth assay can include exposing the perturbed strain and control strain to a stressor, such as an antibiotic. In some embodiments, fitness values (w) can be calculated using the standard Malthusian Fitness Equation using the formula ω=ln(N_{E1}*100²/N_{E0})/ln(N_{C1}*100²/N_{C0}), where the variables are defined as follows: “N”—Colony Forming Units (CFU), “E”—experimental strain, “C”—control strain, “1”—after exposure to the stressor, and “0”—before exposure to the stressor. Other known methods for calculating bacterial fitness can be used.

In some embodiments, expected fitness values (ω_{E}) for strains with perturbation of two or more genes can be calculated, assuming a multiplicative model as follows:

$${ω}_{E} = {\prod }_{i = 1}^{n} {ω}_{i}$$

where n expands to all sets of genes perturbed. For example, ω_{E} of perturbed strainMG1655-dzf (E. coli with inhibited expression of genes dfp, zwf, and frr) can be calculated as the product of fitness from each individual gene perturbation (ω_{d}*ω_{z}*ω_{f}). In some embodiments, epigenetic epistasis can be calculated as the difference between measured fitness (ω) and expected fitness (ω_{E}). In some embodiments, it is then determined whether any calculated epigenetic epistasis is significant. For example, it can be determined whether epigenetic epistasis values deviate from the null hypothesis (no epistasis) using standard error to determine the 95% confidence interval and subsequently performing a z-test (assuming two-tailed distribution) to obtain P-values. It will be apparent that certain modifications can be made to this epistasis calculation, which is provided merely as an example.

In some embodiments, the one or more biological tools capable of modulating expression of the gene or group of genes in a single bacterium can be, but are not limited to, a CRISPR/Cas (Clustered Regularly Interspaced Short Palindromic Repeat/CRISPR associated protein) system, a transcription activator-like effector nuclease (TALEN) system, a zinc-finger protein system, a synthetic polyamide system, a meganuclease system, a morpholino-based system, a peptide nucleic acid-based system, a locked nucleic acid-based system, and RNA interference-based system. These tools are discussed in detail herein, although other biological tools known in the art capable of modulating gene expression in bacteria can also be used and are contemplated herein.

In some embodiments, the biological tools of use herein can be designed to specifically target a single gene or a group of two or more genes, resulting in modulation of gene expression. In some embodiments, expression of the single gene or group of genes can be inhibited. In other embodiments, expression of the single gene or group of genes can be activated. In embodiments where expression of a group of genes is modulated, expression of some genes in the group can be inhibited while expression of other genes in the group can be activated.

In some embodiments, a single bacterial response gene can be targeted. In some embodiments, combinations of bacterial stress response genes and/or bacterial essential genes can be targeted. Bacterial stress response genes can include, but are not limited to, E. coli genes mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and analogous or homologous genes to these found in other bacteria. In certain embodiments, targeting RNA sequences can be designed to target one or more of mutS, soxS, tolC, acrA, recA, dinB, and marA, or analogous of homologous genes thereof. The major functions for the gene products of each of mutS, soxS, tolC, acrA, recA, dinB, and marA are listed in Table 1. Other genes associated with bacterial fitness can also be targeted. Bacterial essential genes are those genes that are indispensable to support bacterial cell life, and constitute a minimal gene set for a living cell. Such genes are well known, and can be identified for many organisms in various databases, such as the Database of Essential Genes (available at essentialgene.org; Zhang, R., et al. (2004) Nucleic Acids Res, 32:D271-271), the Cluster of Essential Gene Database (available at cefg.uestc.edu.cn/ceg; Ye, Y N, et al. (2013) BMC Genomics. 14:769), and OGEE (Online Gene Essentiality database; available at ogee.medgenius.info; Chen et al. (2017) Nucleic Acids Res, 45(D1):D940-D944). In some embodiments, the bacterial genes can include, but are not limited to, the E. coli genes dfp (essential for Coenzyme A synthesis), topA (essential for relaxing DNA supercoiling), zwf (a key glycolysis enzyme) and frr (ribosome recycling factor), and analogous or homologous genes to these found in other bacteria. In some embodiments, the bacterial conserved genes are highly preserved across all bacterial genomes.

In some embodiments, those genes of other bacteria that are either analogous or homologous to the E. coli genes described herein can be targeted for modulation in the gene's host.

In some embodiments, a high-throughput approach can be employed to identify a single gene or a group of genes whose expression affects bacterial fitness and/or adaptation to antibiotics.

TABLE 1
Functions of select bacterial stress response gene products.

| Gene | Protein | Function |
|---|---|---|
| mutS | MutS | Combines with MutH and MutL to form the MutHLS complex, which is directed by |
| | | methylation to repair DNA-DNA mismatches. MutS binds to mismatched DNA and |
| | | directs MutH to cleave the unmethylated strand, allowing for other enzymes to repair the |
| | | mismatch. In this way, MutS serves to maintain the genomes' status quo. |
| soxS | SoxS | Dual transcriptional regulator of the superoxide stress response. Binds to common |
| | | recognition motifs to regulate expression of genes involved in the superoxide regulon. |
| | | SoxS and MarA share 49% homology, and bind to similar DNA elements such as the Mar- |
| | | Sox-Rob box. SoxS also regulates expression of some genes controlled only by the Sox box |
| | | independent of MarA |
| tolC | TolC | Part of multiple multidrug efflux pump systems, including TolC-AcrAB. The TolC |
| | | trimeric protein acts as an outer membrane porin to shuttle hydrophobic and amphipathic |
| | | molecules outside of the cell. TolC also binds to the periplasmic component of AcrAB |
| acrA | AcrA | Part of the TolC-AcrAB multidrug efflux pump. AcrA is a periplasmic protein which |
| | | complexes with the inner membrane protein AcrB. It acts as a secondary transporter of |
| | | molecules from AcrB to TolC as they are shuttled outside the cell. |
| recA | RecA | Induces the SOS response by cleaving LexA dimers. These dimers bind to the SOS box |
| | | to constitutively inhibit genes involved in the SOS response. DNA damage (and double |
| | | strand breaks in particular) activates RecA to cleave LexA, thus freeing repression of the |
| | | SOS response. |
| dinB | DNA | Polymerase which lacks proofreading capacity, making it more prone to introducing errors |
| | polymerase IV | during replication and thus creates spontaneous mutations. The polymerase has particular |
| | | affinity towards misalignments and DNA lesions. |
| marA | MarA | Multiple Antibiotic Resistance protein which acts as a dual transcriptional regulator |
| | | of at least 60 other genes which play roles in protection against antimicrobial stressors. |
| | | Shares homology with SoxS, and binds to similar DNA elements such as the Mar-Sox- |
| | | Rob box. MarA also regulates expression of some genes controlled only by the Mar box |
| | | independent of SoxS. |

In some embodiments, combinations of genes that result in negative epigenetic epistasis can be identified utilizing a high-throughput screening approach. In some embodiments, a high-throughput screening approach utilizes a large number of biological tools, each specific for a unique (i.e., single) gene, to cause modulations in gene expression of two or more genes in various combinations, resulting a range of perturbed strains being generated simultaneously. In some embodiments, the range of perturbed strains can then be tested for any changes in epigenetic epistasis.

In some embodiments, gene combinations resulting in reduced bacterial fitness and negative epigenetic epistasis are selected. In accordance with these embodiments, the genes of the selected combinations can serve as targets for antibiotic drug discovery. In certain embodiments, a goal is to to identify a single antibiotic or a group of antibiotics that target each gene of the selected combination. In other embodiments, the genes of the selected combination can be targeted using the methods, compositions, and methods of use described herein.

### Methods for Altering Bacterial Fitness.

In one aspect, methods for altering bacterial fitness of a bacterium are contemplated. In some embodiments, an alteration in bacterial fitness results in negative epistasis. In some embodiments, bacterial fitness of a bacterium is altered by modulating gene expression of a single gene. In some embodiments, bacterial fitness of a bacterium is altered by modulating gene expression of a group of at least 2 genes in the bacterium. In other embodiments, bacterial fitness of a bacterium is altered by modulating a group of at least three genes in the bacterium. In yet other embodiments, bacterial fitness of a bacterium is altered by modulating a group of at least four genes in the bacterium. In some embodiments, methods for altering bacterial fitness of a bacterium provided herein lead to death of the bacterium. In other embodiments, the methods for altering bacterial fitness of a bacterium provided herein lead to the slowing or prevention of development of adaptive resistance to antibiotics in the bacterium. In some embodiments, slowing or prevention of development of adaptive resistance to antibiotics results from a reduced rate or halt of evolution of adaptive resistance in the bacterium caused by an negative epigenetic epistatic effect caused by the modulation of gene expression of a group of genes. In some embodiments, a group of genes includes at least two, at least three, or at least four genes.

In some embodiments, the group of at least two, at least three, or at least four genes includes, but is not limited to, at least one bacterial stress response gene, at least one bacterial essential gene, or a combination of both at least one bacterial stress response gene and at least one bacterial essential gene. For example, in a group of three genes, all three targeted genes can be stress response genes, or two genes can be stress response genes and one gene can be an essential gene.

In some embodiments, expression of genes can be modulated by either activating expression of the genes, inhibiting expression of the genes, or activating expression of at least one of the genes in a group of genes while inhibiting expression of the others genes in the group, or vice versa. For example, in a group of three genes, expression of one may be inhibited, while expression of the other two is activated. This can occur whether the genes are bacterial response genes, conserved genes, or bacterial essential genes.

In some embodiments, the group of genes includes at least one bacterial stress response gene selected from the group of E. coli genes mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, although other bacterial stress response genes may also be targeted for modulation, and are contemplated herein.

In some embodiments, the group of genes includes at least one bacterial essential gene selected from lexA, recA, dfp, zwf, topA, frr, and rfp although other bacterial essential genes may also be targeted for modulation, and are contemplated herein.

In some embodiments, at least three genes selected from mutS, soxS, tolC, recA, dfp, zwf, topA, frr, and rfp are targeted for modulation of gene expression. In other embodiments, a group of at least three genes targeted for modulation of gene expression is selected from mutS, soxS, and to/C; mutS, soxS, and recA; mutS, tolC, and recA; soxS, to/C, and recA; dfp, zwf, and topA; dfp, zwf, and frr; dfp, topA, and frr; mutS, soxS, tolC, and recA; dfp, zwf, topA, and frr; and mutS, soxS, topA, and frr. In other embodiments, a group of at least two genes targeted for modulation of gene expression is selected from mutS and soxS; soxS and recA; dfp and tolC; zwf and topA; zwf and frr; and topA and frr.

In some embodiments, at least some genes of a group of genes are not known to interact with one another. In other embodiments, no genes of the group of genes are known to interact with one another.

In some embodiments, gene expression is modulated at the transcriptional level. In other embodiments, gene expression is modulated at the post-transcriptional level. In yet other embodiments, some genes of a group of genes can be modulated at the transcriptional level while other genes of the group can be modulated at the post-translational level. This includes a single gene of the group being modulated at one level, while all other genes of the group are modulated on another level.

In some embodiments, gene expression is modulated at the transcriptional level by delivering to a bacterium one or more of a CRISPR/Cas system, a transcription activator-like effector (TALE) system, a zinc-finger protein system, a synthetic polyamide system, and a meganuclease system. In some embodiments, these systems are engineered to specifically target a single gene or each gene of a group of genes.

### CRISPR/Cas to Modulate Gene Expression.

In some embodiments, gene expression is modulated by a CRISPR/Cas system. The Clustered Regularly Interspaced Short Palindromic Repeat/CRISPR-Associated (CRISPR/Cas) nuclease system is an engineered nuclease system based on a bacterial system. The CRISPR/Cas system can be used for genome editing, gene inhibition (CRISPRi), and gene activation (CRISPRa). It is based part of the adaptive immune response present in many bacteria and archaea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 nuclease to a region homologous to the crRNA in the target DNA called a “protospacer.” Cas9 cleaves the DNA to generate blunt ends at the DSB at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single-guide RNA,” or sgRNA, or gRNA), and the crRNA equivalent portion of the single guide RNA can be engineered to guide the Cas9 nuclease to target any desired sequence. Thus, the CRISPR/Cas system can be engineered to create a double strand break at a desired target in a genome, and repair of the DSB can be influenced by the use of repair inhibitors to cause an increase in error prone repair.

More recently, the Cas9 has been modified to render both catalytic domains (RuVC and HNH) of the protein inactive, resulting in a catalytically-dead Cas9 (dCas9). The dCas9 is unable to cleave DNA, but maintains its ability to specifically bind to DNA when guided by a guide RNA (gRNA). This allows the CRISPR/dCas system to be used as a sequence-specific, non-mutagenic gene regulation tool.

In some embodiments, the CRISPR/Cas system effective to modulate gene expression includes a catalytically-dead CRISPR-associated (dCas) protein and at least two, at least three, or at least for guide RNA (gRNA) molecules. In some embodiments, each of the gRNA molecules includes a CRISPR-associated (Cas) protein binding site and a targeting RNA sequence. In some embodiments, each of the gRNA molecules specifically target one gene of the group of genes. This is possible by designing a gRNA to include a targeting nucleic acid sequence that is complementary to a target sequence on the target gene. In some embodiments, the target sequence of the target gene can be a regulatory region of the gene. Methods for designing and generating gRNAs are known. In some embodiments, this specificity allows for species and even strain specificity, allowing for targeting gen modulation only in the desired bacterial species or strain.

In some embodiments, each of the gRNA molecules specifically binds to its target sequence in the single gene, or in each of the genes in the group of genes, which then guide the dCas9 to the target sequence, where it can interfere with transcription elongation by blocking RNA polymerase or transcription initiation by blocking RNA polymerase binding and/or transcriptions factor binding. This CRISPR/dCas interference (CRISPRi) system is highly efficient in suppressing genes, as it is specific, with minimal off-target effects, and is multiplexable, thus allowing for the interference with multiple genes.

In some embodiments, the targeting nucleic acid sequence can be from 20 to 30 base pairs in length. In some embodiments, the targeting nucleic acid sequence can be about 20 base pairs in length. In some embodiments, the targeting nucleic acid sequence can be complementary to the target nucleic acid sequence and bind specifically to the target nucleic acid sequence.

The binding of the gRNA to the target sequence of the target gene localizes the dCas to the target gene via the Cas protein binding site.

In some embodiments the gRNA molecules include a targeting nucleic acid sequence that is the complement of a target nucleic acid sequence of a target gene. In some embodiments, the target nucleic acid sequence of a target gene is selected from SEQ ID NO: 1-18. Target nucleic acid sequences and their associated target genes are presented in Table 2.

In some embodiments, the CRISPR/Cas system also includes a transcriptional effector molecule. In some embodiments, the transcriptional effector is selected from DNA methylase, histone acetylase, and RNA polymerase ω-subunit, although this list is not exclusive, and other effector molecules are contemplated herein. In some embodiments, the transcriptional effector molecule is the RNA polymerase ω-subunit. By attaching, or fusing, the ω-subunit to the dCas9, the CRISP/dCas system can activate gene expression (CRISPRa).

TABLE 2
Target nucleic acid sequences and
their associated target genes.

| Target | Activation/ | | SEQ ID |
|---|---|---|---|
| Gene | Inhibition | Target Sequence | NO: |
| mutS | Inhibition | ctgctgcatcatgggcgtat | 1 |
| soxS | Inhibition | ctacatcaatgttaagcggc | 2 |
| tolC | Inhibition | ggctcaggccgataagaatg | 3 |
| acrA | Inhibition | agcatcagaacgaccgccag | 4 |
| recA | Inhibition | taccaaattgtttctcaatc | 5 |
| dinB | Inhibition | attgtcgcgcatctccactg | 6 |
| marA | Inhibition | ccagtccaaaatgctatgaa | 7 |
| mutS | Activation | gcaagtacgcaaaattgtat | 8 |
| soxS | Activation | gcgtttcgccacttcgccgg | 9 |
| tolC | Activation | agcagtcatgtgttaaattg | 10 |
| acrA | Activation | gagccacatcgaggatgtgt | 11 |
| recA | Activation | ccgtgatgcggtgcgtcgtc | 12 |
| dinB | Activation | gcaaaagctggataagcagc | 13 |
| marA | Activation | gttttgttcaatgcgatgca | 14 |
| dfp | Inhibition | gtgataaaatcgccaacttc | 15 |
| topA | Inhibition | ctggcaacgagttaccgata | 16 |
| zwf | Inhibition | gtatacttgtaattttctta | 17 |
| frr | Inhibition | agccctgattaaacatatta | 18 |

In some embodiments, a CRISPR/Cas system that specifically targets a group of bacterial genes and causes modulation of their expression, altering bacterial fitness in a bacterium can be delivered without any carrier, for example, naked. In other embodiments, the system can be incorporated into one or more nanoparticles for delivery to a subject. In some embodiments, the one or more nanoparticles can include on its surface one or more target bacteria-specific antibodies, one or more target bacteria specific cell-penetrating peptides, or a combination thereof. In some embodiments, a single nanoparticle includes synthetic polynucleotide analogs specific for each target gene. In other embodiments, polynucleotides targeting two or more target genes can be included in two or more nanoparticles.

Some embodiments provide expression modulated by a CRISPR/Cas system expressible from one or more vectors. In some embodiments, components of the CRISPR/Cas system are expressed from a dual CRISPR/Cas vector system including a first vector that encodes a Cas protein (e.g., dCas9), and at least one additional vector that encodes the gRNAs. In some embodiments, the dCas protein is encoded by a first nucleic acid sequence, and each of the gRNA molecules are encoded by a separate nucleic acid sequence. In some embodiments, a single additional vector encodes all gRNAs. In another embodiment, two or more additional vectors encode all gRNAs. For example, where the group of target genes includes two genes, two additional vectors encode one gRNA each; where the group of target genes includes three genes, two gRNAs can be encoded by one additional vector, and the third gRNA can be encoded by a second additional vector; or, where the group of target genes includes three genes, three additional vectors encode one gRNA each. In some embodiments, the first vector encoding the Cas protein also encodes at least one gRNA, while at least one additional vector encodes the remaining gRNAs.

In some embodiments, the components of the CRISPR/Cas system are expressed from a single vector, where a single vector encodes a Cas protein (e.g., dCas9) and all gRNAs.

In some embodiments, the vector that encodes the Cas protein, whether a single vector system or a dual vector system, also encodes a transcriptional effector molecule. In some embodiments, the nucleic acid sequence encoding the Cas protein and nucleic acid sequence encoding the transcriptional effector molecule are arranged in the vector so that when expressed, the Cas protein and transcriptional effector molecule form a fusion polypeptide. In some embodiments, the transcriptional effector is selected from DNA methylase, histone acetylase, and RNA polymerase subunit, although this list is not exclusive, and other effector molecules are contemplated herein In some embodiments, the transcriptional effector molecule is the RNA polymerase ω-subunit.

Methods for producing both single vector and dual vector CRISPR/Cas systems are known. Representative examples of such methods, and vectors that may be used in such methods, are described in the Examples section.

In some embodiments described throughout this disclosure, the Cas protein is Cas9. In other embodiments, the dCas protein is dCas9.

In some embodiments, a vector-expressible CRISPR/Cas system is encapsulated in one or more nanoparticles. In some embodiments, nanoparticles carrying the CRISPR/Cas system include at least one cell-cell-specific targeting ligand for the bacterium selected from an antibody and a cell-penetrating peptide. In some embodiments, a vector-expressible CRISPR/Cas system is incorporated into a bacteriophage capable of transferring the vector expressible CRISPR/Cas system to a target bacterium. In some embodiments, a vector-expressible CRISPR/Cas system is incorporated into a donor cell capable of transferring the vector expressible CRISPR/Cas system to a target bacterium. Donor cells can include but are not limited to other bacteria.

### Other Translational Control Systems

In some embodiments provided herein, expression of the group of genes is modulated by a transcription-activator-like effector (TALE) system. The TALE Nuclease (TALEN) system originated from the study of bacteria of the Xanthomonas genus. The bacteria, which are pathogens of crop plants, were found to secrete effector proteins (transcription activator-like effectors; TALEs) to the cytoplasm of plant cells, which then bind DNA and activate the expression of their target genes via mimicking the plan cell's transcription factors. TALE proteins are composed of a central domain responsible for DNA binding, a nuclear localization signal, and a domain that activates the target gene transcription. The DNA-binding domain includes monomers, each of which binds one nucleotide in the target nucleotide sequence. Monomers are tandem repeats of 34 amino acid residues, two of which are located at positions 12 and 13 and are highly variable (repeat variable diresidue, RVD), and it is these RVDs that are responsible for the recognition of a specific nucleotide. In some embodiments, a TALEs is designed to specifically target a single gene of the group of genes being targeted. In some embodiment, a TALE is designed to recognize 15-20 base pairs of the target gene. In some embodiments, a TALE system includes two or more TALE proteins that each selectively bind to a single target gene of the group of target genes. In some embodiments, a transcriptional effector molecule is fused to each of the TALE proteins, allowing for activation or inhibition of gene expression, depending on the nature of the transcriptional effector molecule.

In some embodiments, a zinc-finger protein system, a synthetic polyamide system, or a meganuclease system can be used to modulate target gene expression. In some embodiments, each system includes two or more, three or more, or four or more components, each specifically targeting a single gene of a group of genes. These systems are known in the art, and it will be recognized by one of skill in the art how to design and prepare such systems to alter bacterial fitness in light of this disclosure.

In some embodiments, gene expression is modulated at the post-transcriptional level by delivering to a bacterium one or more of a morpholino-based system, a peptide nucleic acid-based system, and a locked nucleic acid-based system. In some embodiments, these systems are engineered to specifically target each gene of a group of genes.

### Synthetic DNA Analogs

In some embodiments, gene expression of a group of genes in a bacterium is modulated by a peptide nucleic acid (PNA)-based system. PNAs are DNA analogs in which the phosphate backbone has been replaced by (2-aminoethyl) glycine carboyl units that are linked to the nucleotide bases by the glycine amino nitrogen and methylene carbonyl linkers. The backbone is thus composed of peptide bonds linking the nucleobases. Because the PNA backbone is composed of peptide linkages, the PNA is typically referred to as having an amino-terminal and a carboxy-terminal end. However, a PNA can be also referred to as having a 5′ and a 3′ end in the conventional sense, with reference to the complementary nucleic acid sequence to which it specifically hybridizes. The sequence of a PNA molecule is described in conventional fashion as having nucleotides G, U, T, A, and C that correspond to the nucleotide sequence of the PNA molecule. Such polynucleotides can be synthesized, for example, using an automated DNA synthesizer. Typically, PNAs are synthesized using either Boc or Fmoc chemistry. PNAs and other polynucleotides can be chemically derivatized by methods known to those skilled in the art. For example, PNAs have amino and carboxy groups at the 5′ and 3′ ends, respectively, that can be further derivatized. Custom PNAs can also be synthesized and purchased commercially.

In other embodiments, gene expression of a group of genes in a bacterium is modulated by a polynucleotide analog that is not a PNA, such as LNAs, morpholinos, bridged nucleic acids (BNAs), phosophorothioate oligonucleotides, phosphorodiamidate oligonucleotides, and 2′-O-methyl-substituted RNA, although other synthetic polynucleotide analogs can also be used. In some embodiments, the synthetic polynucleotide analogs can be LNAs. LNA polynucleotides are modified RNA nucleotides. The ribose moiety of an LNA polynucleotide is modified with an extra bridge connecting the 2′ and 4′ carbons. The bridge “locks” the ribose the 3′-endo structural conformation, which is often found in the A-form of DNA or RNA. The locked ribose conformation enhances base stacking and backbone pre-organization. This significantly increases the thermal stability (melting temperature) of oligonucleotides. Due to their constrained backbone, LNA polynucleotides have a high affinity for single-stranded DNA or RNA. LNA bases can be included in an LNA backbone, 2′-O-methyl RNA backbone, 2′-methoxyethyl RNA backbone, 2′-fluoro RNA DNA backbone, or a DNA backbone. LNA polynucleotides can utilize either a phosphodiester or phosphorothioate backbone. In addition to high affinity, LNA polynucleotides display high in vivo stability and slower renal clearance.

In other embodiments, the synthetic polynucleotide analog can be a BNA polynucleotide. BNA monomers can contain a five-, six-, or even a seven-membered bridged structure with a fixed C3′-endo sugar puckering. The bridge is synthetically incorporated at the 2′, 4′-position of the ribose to afford a 2′, 4′-BNA monomer. An increased conformational inflexibility of the sugar moiety in BNA oligonucleotides results in a gain of high binding affinity with complementary single-stranded RNA and/or double-stranded DNA. BNAs are useful for the detection of short DNA and RNA targets, are capable of single nucleotide discrimination, and are resistant to exo- and endonucleases, resulting in high stability for in vivo and in vitro applications.

In certain embodiments, the synthetic polynucleotide analog can be a 2′-O-methly polynucleotide. In a 2′-O-methly polynucleotide, a methyl group replaces a hydrogen atom in the 2′-hydroxyl group in the ribose ring of RNA, imparting nuclease resistance and inhibiting RNAse-H activation, leaving target RNA intact. Although the 2′-O-methyl modification is insensitive to endonucleases, it is still partially susceptible to exonuclease degradation. By combining PS linkages and 2′-O-methyl nucleotides, much greater in vivo stability can be achieved.

In some embodiments, the synthetic polynucleotide analog can be a peptide nucleic acid (PNA) polynucleotide. A PNA polynucleotide is a polypeptide with N-(2-aminoethyl)glycine as the unit backbone. Like DNA and RNA, PNA also selectively binds to complementary nucleic acid. Having a neutral backbone due to the replacement of the phosphates in the backbone, the binding between PNA and RNA is stronger than that between DNA and RNA or RNA and RNA due to the lack of electrostatic repulsion. The neutral backbone also results in the binding being practically independent of salt concentration. In addition to having increased binding affinity, PNAs are known to bind RNA with increased specificity, with sensitivities capable of discriminating against a single base pair mismatch. This is a significant improvement over strategies such as RNAi utilizing siRNA or miRNA. Since PNA is structurally markedly different from DNA, PNA is very resistant to both proteases and nucleases, and is not recognized by the hepatic transporter(s) recognizing DNA.

In some embodiments, a synthetic polynucleotide analog system provided herein comprises at least two, at least three, or at least four synthetic polynucleotide analog molecules, each being an antisense synthetic polynucleotide analog capable of specifically targeting and hybridizing with a target sequence of a target gene. In some embodiments, each synthetic polynucleotide analog of the polynucleotide analog system specifically targets a unique target gene in a group of genes. In some embodiments, the target domain, and thus the antisense (complementary) synthetic polynucleotide analog, can be about 5 to about 20 nucleotides in length. The length of the synthetic polynucleotide analog can be optimized for the specific intended use and target domain. In some embodiments, the target sequence and its complementary synthetic polynucleotide can have a length of 5 to 20 nucleotides.

In some embodiments, the antisense synthetic polynucleotide can be a peptide nucleic acid (PNA).

In some embodiments, a synthetic polynucleotide analog can be covalently coupled to a cell penetrating peptide (CPP). Coupling a CPP to the synthetic polynucleotide analog can improve cytosolic delivery of the synthetic polynucleotide analog. CPPs represent short polypeptide sequences of about 10 to about 30 amino acids which can cross the plasma membrane of bacterial cells.

In some embodiments, a synthetic polynucleotide analog system that specifically targets a group of bacterial genes and causes modulation of their expression, and thus alters bacterial fitness in a bacterium's can be incorporated into one or more nanoparticles for delivery to a subject. In some embodiments, the one or more nanoparticles can include on its surface one or more target bacteria-specific antibodies, one or more target bacteria specific cell-penetrating peptides, or a combination thereof. In some embodiments, a single nanoparticle includes synthetic polynucleotide analogs specific for each target gene. In other embodiments, polynucleotides targeting two or more target genes can be included in two or more nanoparticles.

In vitro methods.

In some embodiments, a method described herein can be performed in vitro using any one or combination of the methods described herein, such as modulating gene expression of a group of at least two, at least three, or at least four genes to alter bacterial fitness in a bacterium at the transcriptional level by delivering to the bacterium a CRISPR/Cas system, a transcription activator-like effector (TALE) system, a zinc-finger protein system, a synthetic polyamide system, or a meganuclease system, or at the post-transcriptional level by delivering to the bacterium a synthetic polynucleotide analog system such as a morpholino-based system, a peptide nucleic acid-based system, or a locked nucleic acid-based system. Such in vitro methods can be useful in the study of epigenetic epistasis, for example.

In vivo methods.

In other embodiments, a method described herein can be performed in vivo using any one or combination of the methods described herein, such as modulating gene expression of a group of at least two, at least three, or at least four genes to alter bacterial fitness in a bacterium at the transcriptional level by delivering to the bacterium a CRISPR/Cas system, a transcription activator-like effector (TALE) system, a zinc-finger protein system, a synthetic polyamide system, or a meganuclease system, or at the post-transcriptional level by delivering to the bacterium a synthetic polynucleotide analog system such as a morpholino-based system, a peptide nucleic acid-based system, or a locked nucleic acid-based system. Such in vivo methods can be useful, for example, in killing bacteria, or slowing or preventing development of adaptive resistance to antibiotics in a subject. Doing so can help slow or halt the evolution of antibiotic resistance in certain bacteria, prolonging the effective life existing antibiotics.

In some embodiments, the in vivo and in vitro methods can further include delivering at least one antibiotic to the bacterium. In some embodiments, the at least one antibiotic is selected from Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.; 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefinetazole (CAS Registry No.: 56796-20-4); Cefotetan (CAS Registry No.: 69712-56-7); Cefprozil (CAS Registry No.: 92665-29-7); Lincomycin (CAS Registry No.: 154-21-2); Linezolid (CAS Registry No.: 165800-03-3); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives. This list is not intended to be limiting, and other antibiotics can be selected and are contemplated herein.

### CRISPR/Cas Systems

Certain embodiments provide a CRISPR/Cas system for altering bacterial fitness of a bacterium. Such a system works to alter bacterial fitness and cause negative epigenetic epistasis as described supra. In some embodiments, a CRISPR/Cas system for altering bacterial fitness of a bacterium includes a catalytically-dead CRISPR-associated (dCas) protein, and at least two, and least three, or at least four guide RNA (gRNA) molecules, wherein each of the gRNA molecules include a CRISPR-associated (Cas) protein binding site and a targeting RNA sequence specific for a unique gene of the bacterium. In some embodiments, each of the gRNA molecules of the system specifically target a single (i.e., unique) gene. In some embodiments, the gRNA molecules of the CRISPR/Cas system target bacterial stress response genes, bacterial essential genes, or a combination thereof. These groups of genes are generally described supra.

In some embodiments, a target stress response gene can be selected from mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and the bacterial essential genes can be selected from dfp, topA, zwf, and frr and any combination thereof.

In other embodiments, the target gene is selected from mutS, soxS, tolC, recA, dfp, zwf, topA, frr, and rfp and any combination thereof.

In some embodiments, the target genes include a group of genes selected from mutS, soxS, and to/C; mutS, soxS, and recA; mutS, tolC, and recA; soxS, tolC, and recA; dfp, zwf, and topA; dfp, zwf, and frr; dfp, topA, and frr; mutS, soxS, tolC, and recA; dfp, zwf, topA, and frr; and mutS, soxS, topA, and frr.

In some embodiments, a CRISPR/Cas system also includes a transcriptional effector molecule attached to the Cas protein. In some embodiments, the Cas protein and the transcriptional effector molecule form a fusion protein. In some embodiments, the Cas protein and the transcriptional effector molecule are linked via a peptide linker. In some embodiments, the transcriptional effector molecule can be selected from a DNA methylase, a histone acetylase, and an RNA polymerase subunit. In some embodiments, the transcriptional effector molecule can be an RNA polymerase subunit.

In some embodiments, a CRISPR/Cas system is encapsulated in one or more nanoparticles or microparticles. In some embodiments, nanoparticles carrying the CRISPR/Cas system include at least one cell-cell-specific targeting ligand for the bacterium selected from an antibody and a cell-penetrating peptide. Compositions of use as nanoparticles are known in the art and contemplated of use in certain embodiments disclosed herein.

### Vector—Expressible CRISPR/Cas Systems

Certain embodiments disclosed herein provide a vector-expressible CRISPR/Cas system for altering bacterial fitness of a bacterium. In accordance with these embodiments, a system can function by expressing the system in the target bacterium and altering bacterial fitness and causing negative epigenetic epistasis as described supra. In some embodiments, a vector-expressible CRISPR/Cas system for altering bacterial fitness of a bacterium includes a first nucleic acid sequence encoding a catalytically-dead CRISPR-associated (dCas) protein, and at least two, at least three, or at least four additional nucleic acid sequences. Each of the additional nucleic acid sequences encodes a unique guide RNA (gRNA) molecule, and each unique gRNA molecule comprise a CRISPR-associated (Cas) protein binding site and a targeting RNA sequence specific for a unique gene of the bacterium.

In some embodiments, the additional nucleic acid sequences of the vector-expressible CRISPR/Cas system encode a unique gRNA that targets a stress response gene or a bacterial essential gene.

In some embodiments, a target stress response gene can be selected from mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, ispA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and the bacterial essential genes can be selected from dfp, topA, zwf, and frr or a combination thereof.

In other embodiments, the target gene is selected from mutS, soxS, tolC, recA, dfp, zwf, topA, frr, and rfp, or a combination thereof.

In some embodiments, the target genes include a group of genes selected from mutS, soxS, and to/C; mutS, soxS, and recA; mutS, tolC, and recA; soxS, tolC, and recA; dfp, zwf, and topA; dfp, zwf, and frr; dfp, topA, and frr; mutS, soxS, tolC, and recA; dfp, zwf, topA, and frr; and mutS, soxS, topA, and frr.

In some embodiments, the first nucleic acid of a vector-expressible CRISPR/Cas system, in addition to encoding a Cas protein, also encodes a transcriptional effector molecule.

In some embodiments, the first nucleic acid molecule encodes a Cas-transcriptional effector molecule. In some embodiments, the transcriptional effector molecule can be selected from a DNA methylase, a histone acetylase, and an RNA polymerase ω-subunit. In some embodiments, the transcriptional effector molecule can be an RNA polymerase ω-subunit. In some embodiments, the Cas protein is dCas9. In some embodiments, the first nucleic acid molecule encodes a dCas9-RNA polymerase ω-subunit fusion protein. This can be accomplished by methods known in the art, such as expressing both polypeptides under the control of the same promoter.

In some embodiments, a vector-expressible CRISPR/Cas system is encapsulated in one or more nanoparticles. In some embodiments, nanoparticles carrying the CRISPR/Cas system include at least one cell-cell-specific targeting ligand for the bacterium selected from an antibody and a cell-penetrating peptide. In some embodiments, a vector-expressible CRISPR/Cas system is incorporated into a bacteriophage capable of transferring the vector expressible CRISPR/Cas system to a target bacterium. In some embodiments, a vector-expressible CRISPR/Cas system is incorporated into a donor cell capable of transferring the vector expressible CRISPR/Cas system to a target bacterium. Donor cells can include but are not limited to other bacteria.

### PNA Systems

Certain embodiments provide a peptide nucleic acid (PNA) system for altering bacterial fitness of a bacterium. In accordance with these embodiments, a system can function to alter bacterial fitness and cause negative epigenetic epistasis as described supra. In some embodiments, a PNA system for altering bacterial fitness of a bacterium includes at least two, at least three, or at least four PNAs, where each PNA includes a sequence of 5 to 20 nucleic acids capable of hybridizing to a target sequence of a single (i.e., unique) gene of the bacterium. In some embodiments, the PNA molecules of the PNA system target bacterial stress response genes, bacterial essential genes, or a combination thereof. These groups of genes are generally described supra.

In some embodiments, a target stress response gene can be selected from mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ifs, lepB, ispA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsD, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and the bacterial essential genes can be selected from dfp, topA, zwf, and frr, or a combination thereor.

In other embodiments, the target gene is selected from mutS, soxS, tolC, recA, dfp, zwf, topA, frr, and rfp, and combinations thereof.

In some embodiments, the target genes include a group of genes selected from mutS, soxS, and to/C; mutS, soxS, and recA; mutS, tolC, and recA; soxS, tolC, and recA; dfp, zwf, and topA; dfp, zwf, and frr; dfp, topA, and frr; mutS, soxS, tolC, and recA; dfp, zwf, topA, and frr; and mutS, soxS, topA, and frr.

In some embodiments, the PNAs of a PNA system can be covalently coupled to a cell penetrating peptide (CPP). Coupling a CPP to a PNA can improve cytosolic delivery of the synthetic polynucleotide analog. CPPs represent short polypeptide sequences of about 10 to about 30 amino acids which can cross the plasma membrane of bacterial cells.

In some embodiments, the PNAs of a PNA system that specifically targets a group of bacterial genes and causes modulation of their expression, and thus alters bacterial fitness in a bacterium's can be incorporated into one or more nanoparticles for delivery to a subject. In some embodiments, the one or more nanoparticles can include on its surface one or more target bacteria-specific antibodies, one or more target bacteria specific cell-penetrating peptides, or a combination thereof.

### Pharmaceutical Compositions and Method of Use.

Some embodiments provide a pharmaceutical composition comprising a CRISPR/Cas system, a vector-expressible CRISPR/Cas system, a PNA system, or other gene regulatory system described herein, or a combination thereof together with a suitable amount of one or more pharmaceutically acceptable vehicles so as to provide a composition for suitable administration to a subject. Suitable pharmaceutically acceptable vehicles are well-known and described in the art

In accordance with these embodiments, pharmaceutical compositions described herein are capable modulating gene expression of a group of genes to alter bacterial fitness in a bacterium. In some embodiments, the pharmaceutical composition in includes a cell-penetrating peptide or a nanoparticle.

In some embodiments, the pharmaceutical composition disclosed herein can further include at least one antibiotic. In some embodiments, the at least one antibiotic is selected from penicillins, cephalosporins, carbacephems, cephamycins, carbapenems, monobactams, aminoglycosides, glycopeptides, quinolones, tetracyclines, macrolides, and fluoroquinolones. In some embodiments, the antibiotic is selected based on the group of genes being target by the CRISPR/Cas system, vector-expressible CRISPR/Cas system, or PNA system of the pharmaceutical composition. In some embodiments, a group of target genes will result in negative epigenetic epistasis resulting in a slowed or halted development of adaptive resistance to a particular antibiotic or family of antibiotics. The antibiotic to be included in the pharmaceutical can be an antibiotic or from a family of antibiotics to which adaptive resistance by the bacterium has been slowed or prevented. In some embodiments, the antibiotic can be a last-resort antibiotic.

In some embodiments, the antibiotic can be administered to the subject separately from the pharmaceutical composition described here, and delivered before or concurrently with the described pharmaceutical composition, or both.

In some embodiments, a pharmaceutical composition described herein can be used to kill bacteria in a subject, or to slow or prevent development of adaptive resistance to antibiotics in bacteria in a subject. In such methods, the pharmaceutical composition is administered at a pharmaceutically effective dose to the subject. A pharmaceutically effective amount, or dose, is an amount of the pharmaceutical composition sufficient to slow or prevent development of adaptive resistance to antibiotics in bacteria in a subject, or to kill bacteria in a subject. In some embodiments, the pharmaceutical composition can be administered by any method known in the art. In certain embodiments, pharmaceutical compositions disclosed herein can be administered orally, intravenously, rectally, vaginally, or intranasally.

### EXAMPLES

The materials, methods, and embodiments described herein are further defined in the following Examples. Certain embodiments are further defined in the Examples herein. It should be understood that these Examples, while indicating certain embodiments, are given by way of illustration only. From the disclosure herein and these Examples, one skilled in the art can ascertain the essential characteristics of this disclosure, and without departing from the spirit and scope thereof, can make various changes and modifications to the disclosure to adapt it to various usages and conditions.

### Example 1—Design and Characterization of Single-Target Gene Modulation Systems

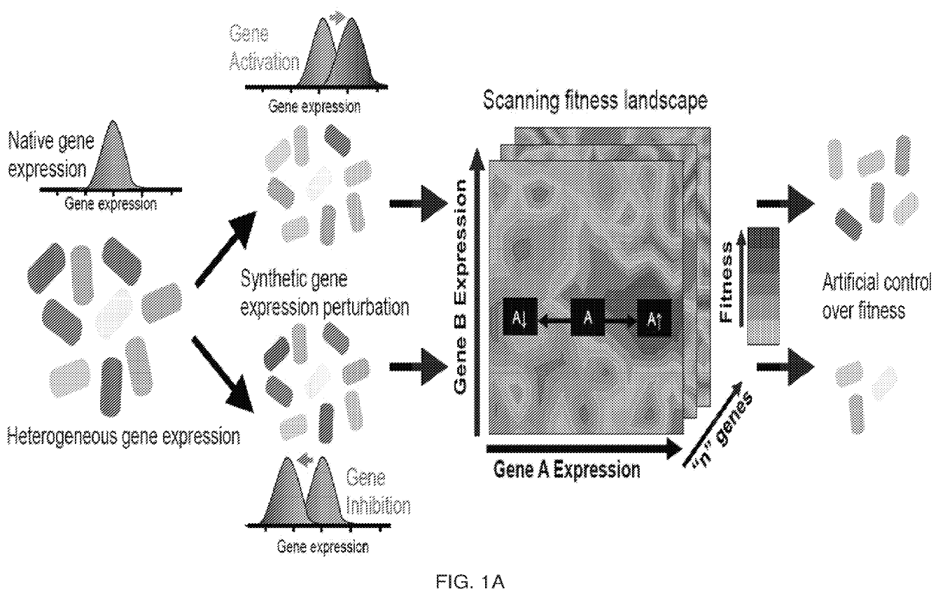

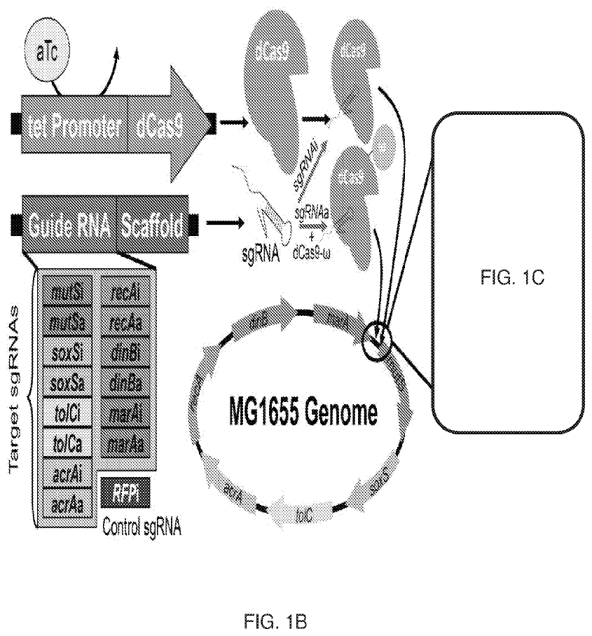

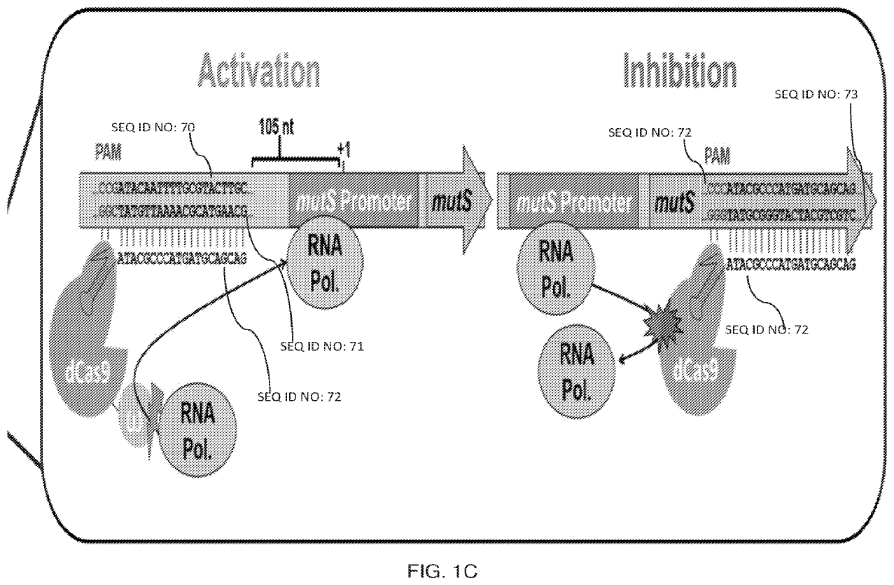

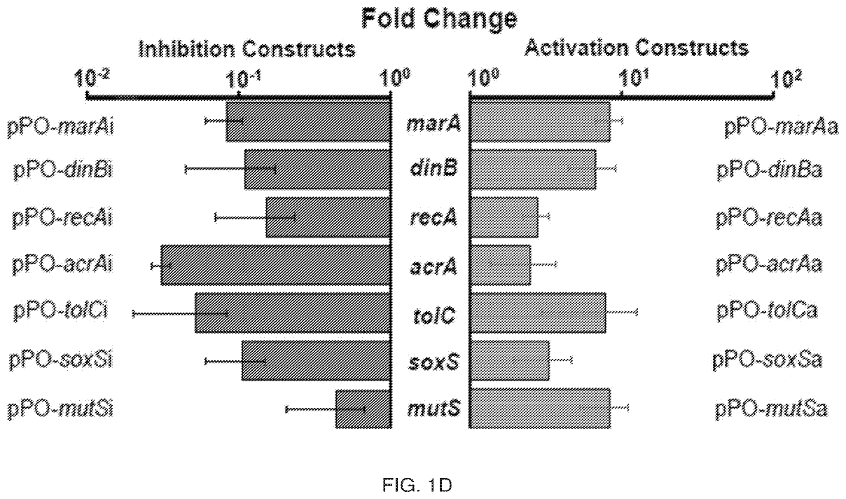

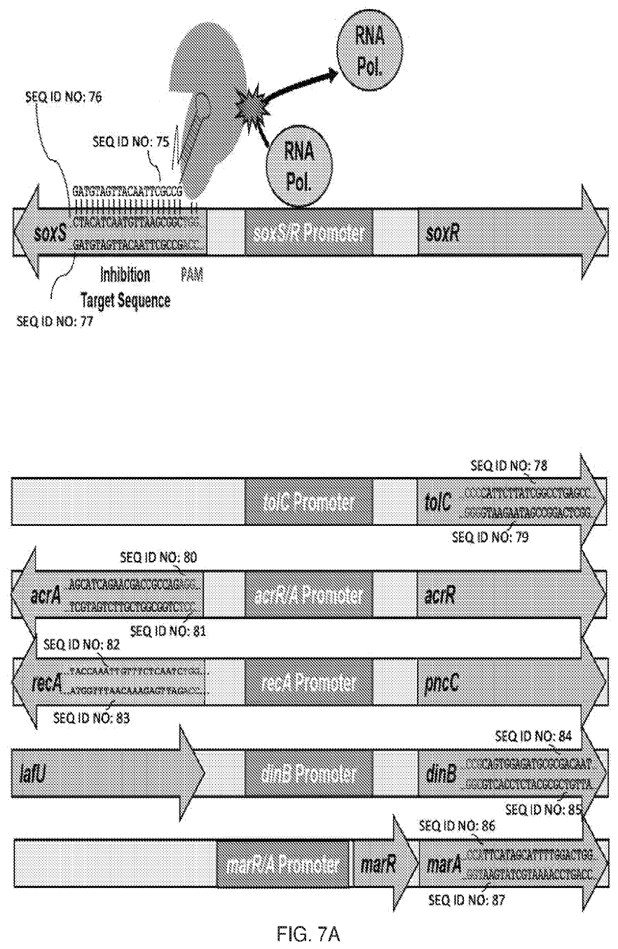

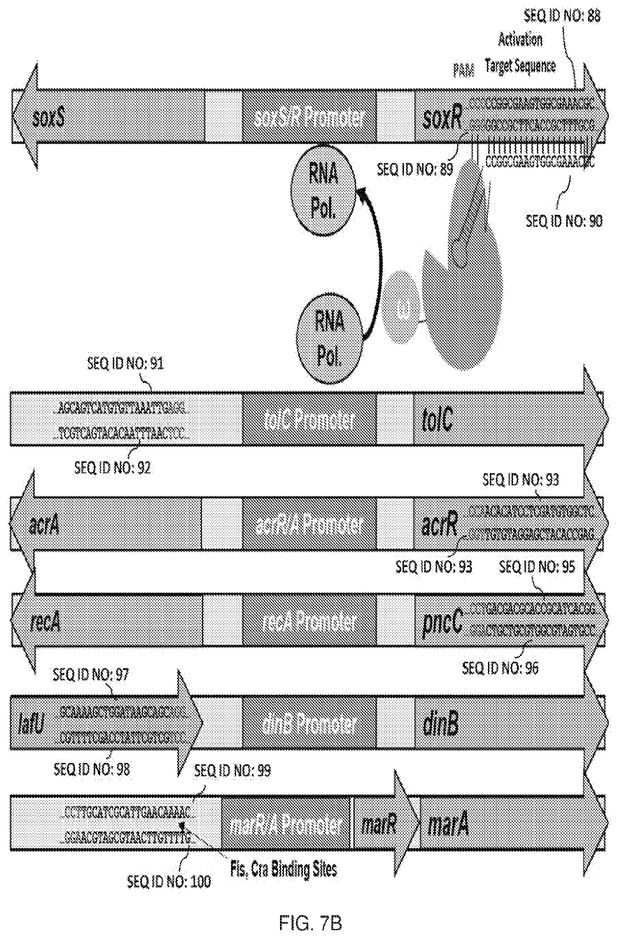

In one example, a set of 14 sgRNA plasmid constructs were designed and synthesized (see Example 7) to inhibit or activate transcription of seven stress-response genes in E. coli, chosen for their known influence on adaptation (FIGS. 1B-1C and FIG. 7). FIGS. 1B-1C illustrate the approach used to modulated gene expression in E. coli. A similar approach can be used to target other stress response genes Similar constructs can be designed and synthesized by any method known in the art.

The sgRNA constructs were named pPO-genei or pPO-genea for inhibition and activation respectively of each given gene (Table 3), and were co-transformed alongside a separate plasmid containing anhydrotetracycline (aTc) inducible dCas9 or dCas9-ω into E. coli strain MG1655. This produced 14 unique experimental modulation strains, designated MG1655-genei or MG1655-genea. Two control strains harboring dCas9 or dCas9-ω plasmids, as well as the control sgRNA construct sgRNA-RFPi (targeting the rfp coding sequence not present in MG1655) were also created (Table 4).

All sgRNAs utilized common promoter and scaffolding elements, but differed in their unique 20 nucleotide (nt) sequence-specific DNA-binding domain. Other similar promoter and scaffolding elements can also be used. Inhibition and activation sgRNAs were coupled in vivo with dCas9 or dCas9-ω respectively to form the final protein-RNA hybrid construct with inherent DNA-binding affinity for the 20 nt sequences of each sgRNA, allowing for specific control of gene expression (FIG. 1C). Activation sgRNAs targeting ≈80-110 nt upstream of the +1 promoter sequences of each gene provided optimal spacing for RNA polymerase to bind to the transcription start site and increase gene expression. Inhibition sgRNAs targeted within the first nt of the genes' open reading frame (ORF) to inhibit transcriptional read-through via a roadblock mechanism. Each sequence was flanked by an “NGG” Protospacer-Adjacent-Motif (PAM) on the 3′end for proper binding of the protein-RNA complex with the target DNA.

FIG. 1C illustrates the binding positions of mutS inhibition and activation constructs. Inhibition constructs prevented RNAP read-through of the target's ORF, while activation constructs recruited RNAP to the promoter region by binding upstream of the +1 sequence.

FIGS. 7A-7B illustrate the inhibition and activation target sequences. FIG. 7A depicts construct-induced gene inhibition (via exclusion of RNA polymerase) for soxS. Corresponding inhibition target sequences are indicated for tolC, acrA, recA, dinB and marA. On the right, a demonstration of construct-induced gene activation (via recruitment of RNA polymerase) is indicated for soxS. Corresponding activation target sequences are depicted for tolC, acrA, recA, dinB and marA. Potential genomic regions of interest are also included. Downstream genes within the same operon include: acrB for acrA, ygiB-D for tolC, recX for recA, yafN-P for dinB and marR/B for marA. The genes mutS and soxS have no other known genes within their operon, although the pphB start site is located 105 nt downstream of mutS indicating potential transcriptional overlap.

TABLE 3
Plasmids used.

| Name | Purpose | Reference or source |
|---|---|---|
| pdCas9 | dCas9 | Addgene Plasmid 44249¹ |
| sgRNA-RFP | | |
| pWJ66 | sgRNA targeting RFP (control) | Addgene Plasmid 44251¹ |
| pPO-dCas9ω | dCas9-ω | Addgene Plasmid 46570² |
| pPO-mCherry | dCas9-ω in 44249 vector | This study |
| | sgRNA targeting RFP (control) expressing aTc | This study |
| | inducible mCherry | |
| pPO-mutSi | sgRNA inhibiting mutS | This study |
| pPO-soxSi | sgRNA inhibiting soxS | This study |
| pPO-tolCi | sgRNA inhibiting tolC | This study |
| pPO-acrAi | sgRNA inhibiting acrA | This study |
| pPO-recAi | sgRNA inhibiting recA | This study |
| pPO-dinBi | sgRNA inhibiting dinB | This study |
| pPO-marAi | sgRNA inhibiting marA | This study |
| pPO-mutSa | sgRNA activating mutS | This study |
| pPO-soxSa | sgRNA activating soxS | This study |
| pPO-tolCa | sgRNA activating tolC | This study |
| pPO-acrAa | sgRNA activating acrA | This study |
| pPO-recAa | sgRNA activating recA | This study |
| pPO-dinBa | sgRNA activating dinB | This study |
| pPO-marAa | sgRNA activating marA | This study |
| pPO-tolCi-acrAi | sgRNA inhibiting tolC and acrA sgRNA | This study |
| pPO-tolCi-acrAi-soxSi | inhibiting tolC, acrA and soxS sgRNA | This study |
| pPO-recAa-dinBa | activating recA and dinB | This study |
| pPO-mutSa-dinBi | sgRNA activating mutS and inhibiting dinB | This study |
| pPO-tolCi-dinBi | sgRNA inhibiting tolC and dinB | This study |

Qi, L. S. et al. (2013) Cell, 152: 1173-1183
Bikard, D. et al. (2013) Nucleic Acids Res., 41: 7429-7437

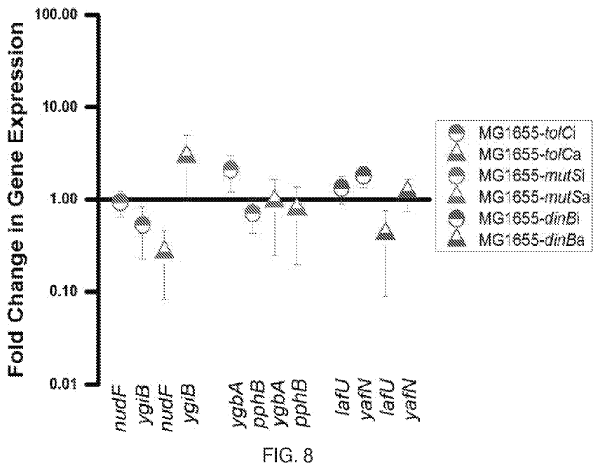

The impact of a subset of these constructs on neighboring genes' expression was quantified, and is depicted in FIG. 8. Perturbing each of these genes can induce changes in expression of additional genes within the transcriptome of E. coli, as governed by the connections through respective gene regulatory networks. As depicted in FIG. 8, qPCR analysis was performed on genes upstream (nudF, ygbA, lafU) and downstream (ygiB, pphB, yafN) of the intended CRISPR perturbation targets to/C, mutS and dinB. Relative fold changes in gene expression from the wild type were quantified using the same approach outlined for FIG. 1D. Some off-target effects were expected; for instance, the dinB activation target overlapped with the 3′ end of the lafU ORF. Consequently, MG1655-dinBa exhibited decreased lafU expression (0.43±0.34 fold) with respect to the control MG1655 strain. Similarly, the activation target for to/C overlapped with the promoter of nudF, making its decrease in expression in MG1655-tolCa (0.27±0.19 fold) expected. Inhibition and activation of to/C caused decreased and increased expression respectively of the downstream gene ygiB by 0.53±0.31 and 2.93±1.97 fold respectively. The slight increase of both ygbA in MG1655-mutSi (2.10±0.90 fold) and yafN in MG1655-dinBi (1.81±0.47 fold) were unexpected, and could indicate potential regulatory overlap of these gene's transcriptional regions (both ygbA and yafN have yet to be fully characterized). Negligible effects were observed in the remainder of the samples. Fold change values are an average of three biological replicates, and represented as Average±Standard deviation.

TABLE 4
Experimental E. coli strains used.

| Name | Plasmids Harbored |
|---|---|
| MG1655 | None |
| MG1655-i MG1655-a | pdCas9 |
| MG1655-rfpi | pPO-dCas9ω |
| MG1655-rfpa | pdCas9 + sgRNA-RFP |
| MG1655-Control | pPO-dCas9ω + sgRNA-RFP |
| MG1655-mCherry | sgRNA-RFP + pdCas9 or pPO-dCas9ω |
| | pPO-sgRNA-mCherry + pdCas9 |
| MG1655-mutSi | pdCas9 + pPO-mutSi |
| MG1655-mutSa | pPO-dCas9ω + pPO-mutSa |
| MG1655-soxSi | pdCas9 + pPO-soxSi |
| MG1655-soxSa | pPO-dCas9ω + pPO-soxSa |
| MG1655-tolCi | pdCas9 + pPO-tolCi |
| MG1655-tolCa | pPO-dCas9ω + pPO-tolCa |
| MG1655-acrAi | pdCas9 + pPO-acrAi |
| MG1655-acrAa | pPO-dCas9ω + pPO-acrAa |
| MG1655-recAi | pdCas9 + pPO-recAi |
| MG1655-recAa | pPO-dCas9ω + pPO-recAa |
| MG1655-dinBi | pdCas9 + pPO-dinBi |
| MG1655-dinBa | pPO-dCas9ω + pPO-dinBa |
| MG1655-marAi | pdCas9 + pPO-marAi |
| MG1655-marAa | pPO-dCas9ω + pPO-marAa |
| MG1655-tolCi-acrAi MG1655- | pdCas9 + pPO-tolCi-acrAi pdCas9 + |
| tolCi-acrAi-soxSi MG1655- | pPO-tolCi-acrAi-soxSi pPO-dCas9ω + |
| recAa-dinBa MG1655-mutSa- | pPO-recAa-dinBa pPO-dCas9ω + pPO- |
| dinBi | mutSa-dinBi |
| MG1655-tolCi-dinBi | pdCas9 + pPO-tolCi-dinBi |

The ability of bacteria to evolve resistance depends on the accessibility of higher-fitness states within a hypothetical “adaptive landscape”, which can be visualized as a multi-dimensional space comprised of the variable expression states of n-by-n genes (analogous to similar adaptive landscapes based on gene mutations) (FIG. 1A). FIG. 1A is a schematic demonstrating the approach to engineer control over the theoretical bacterial fitness landscape. By synthetically modulating an individual gene's native expression by increasing (A↑) or decreasing (A↓) expression using synthetic CRISPR-Cas9 based genetic devices, unique fitness responses can be derived. This approach can further be applied to modulate multiple genes simultaneously to explore this adaptive landscape in n dimensions. Cloning the library of synthetic CRISPR systems described herein into MG1 655 E. coli enabled the engineering of a set of strains in which this adaptive landscape was modulated.

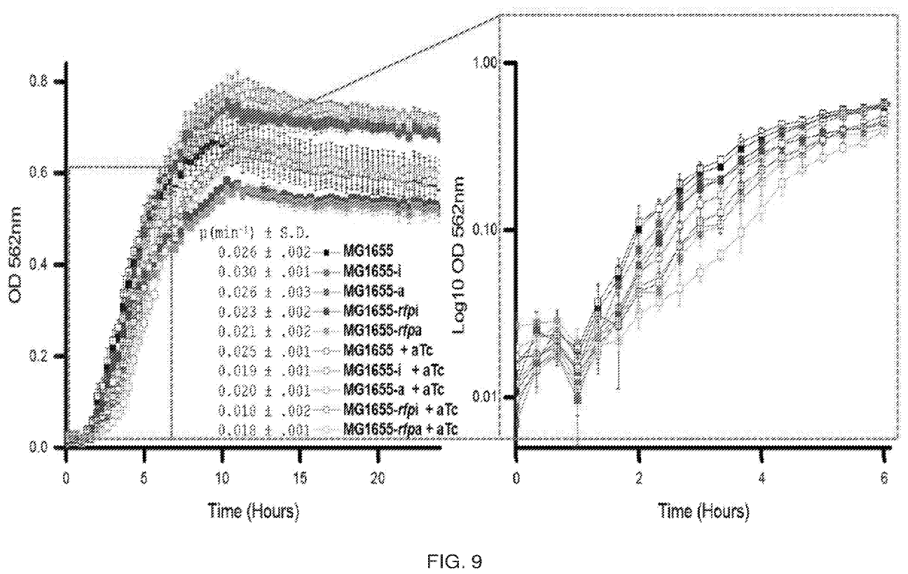

To measure the effects of gene modulation, RT-qPCR was used to quantify the gene expression of each of these strains relative to wild-type MG1655. The results indicate that the strains' expression profiles were modulated, with a range of 32-fold reduction to 8-fold increase in gene expression (FIG. 1D). Optimization of expression modulation was influenced by native gene orientation; for instance, binding of dCas9-ω upstream of the +1 soxS promoter site necessitated overlap with the ORF of soxR, an activator of soxS. Growth tests were also performed to analyze the viability of these strains. No loss of viability that is not intrinsic to growth with two plasmids was observed (FIG. 9). Since MG1655-rfpi and MG1655-rfpa strains demonstrated similar growth characteristics, MG1655-rfpa was used as the control strain in subsequent stress-exposure experiments (referred to hereafter as the MG1655-Control).

For FIG. 1D, gene expression in MG1655 strains harboring dCas9 (inhibition constructs) or dCas9-ω (activation constructs) and sgRNA plasmids (pPO-genei/a) were normalized to housekeeping gene rrsA and calculated relative to wild-type MG1655. genei and gena indicate inhibition and activation respectively of the specific gene. Error bars represent standard deviation (s.d.) of biological triplicates.

Turning to FIG. 9, individual dCas9 (MG1655-i) and dCas9-ω (MG1655-a) plasmids were transformed into MG1655 cells, and were also transformed along with rfp-targeting sgRNA controls (MG1655-rfpi and MG1655-rfpa). Cultures were grown overnight with (dotted curves) and without (solid curves) aTc induction. Growth rates of each strain (presented left of the legend) were calculated using GrowthRates version 1.8, which excludes the estimated lag phase. As expected, a slight decrease in growth was observed for MG1655 strains carrying two plasmids (blue and orange lines), as well as during aTc induction. However, under similar conditions, dCas9 and dCas9-ω plasmids induced no discernable differences in growth rates. A two-tailed t-test indicated no statistically significant differences between the MG1655-i and MG1655-a in the absence (P=0.17) and presence (P=0.36) of aTc. Similarly, no statistically significant difference was found between MG1655-r/Pi and MG1655-rfpa in the absence (P=0.33) and presence (P=0.90) of aTc. Error bars (and s.d.) represent s.d. of biological triplicates. An average growth rate of 0.018 min-1 of MG1655-rfpa strain (which was used as the MG1655-Control strain) corresponds to 38.5 min of doubling time, which gives rise to approximately 7.9 generations during the 5 hours of exponential growth (right panel).

### Example 2—Modulation of Gene Expression Influences Bacterial Growth Characteristics During stress exposure

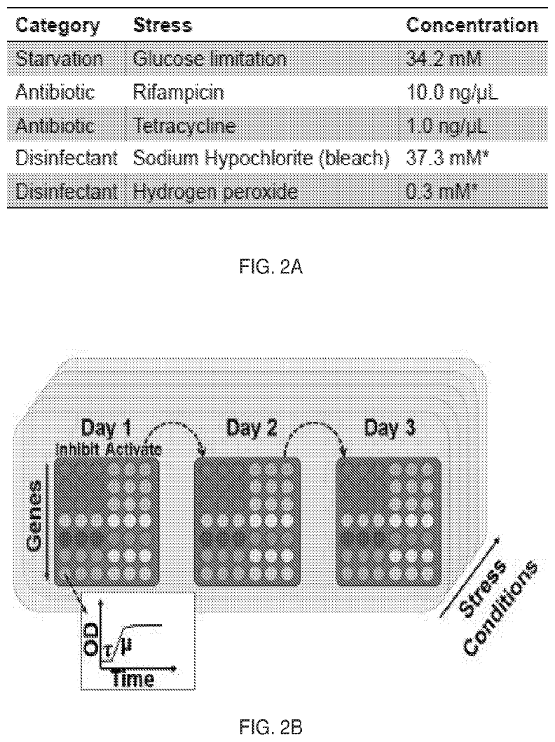

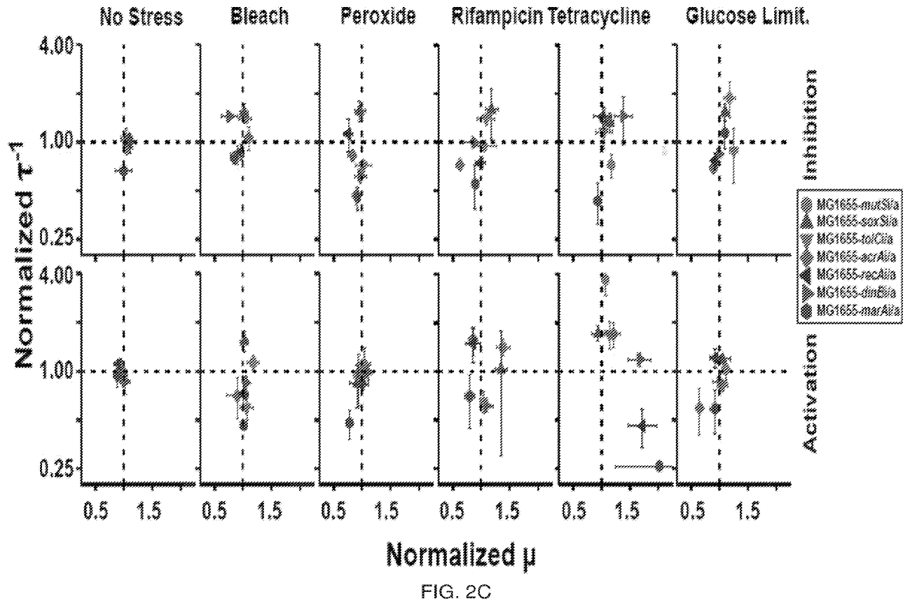

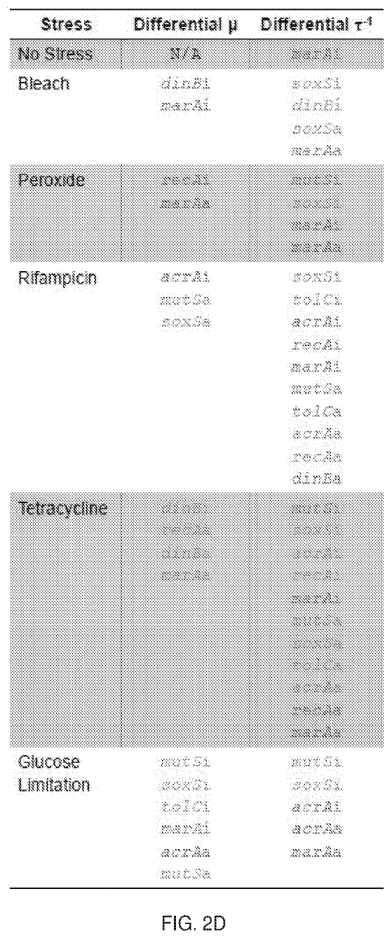

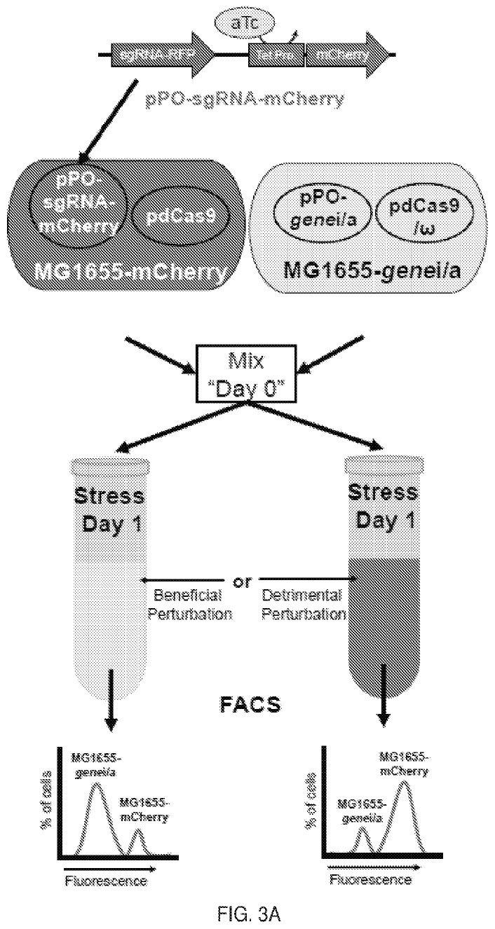

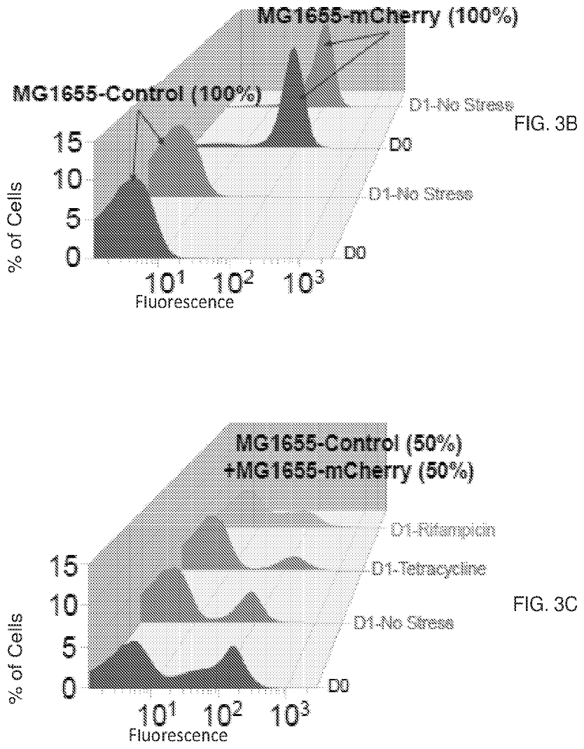

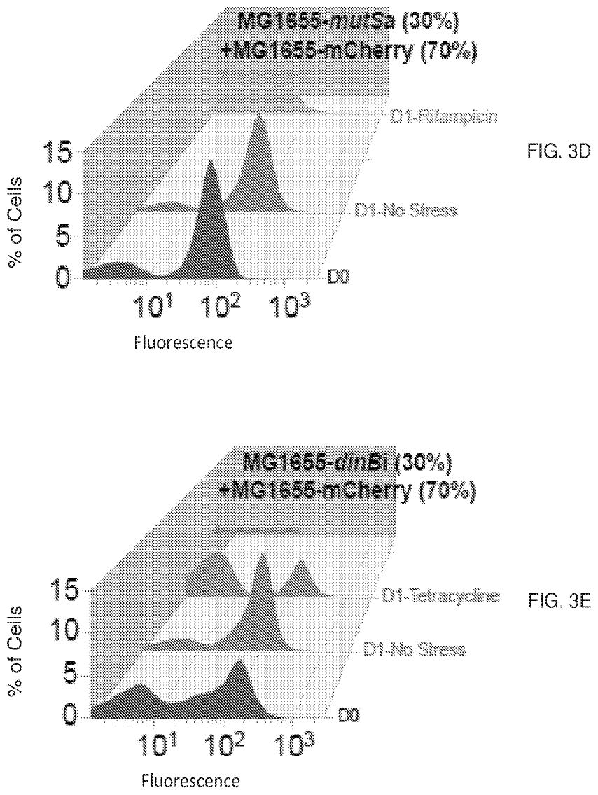

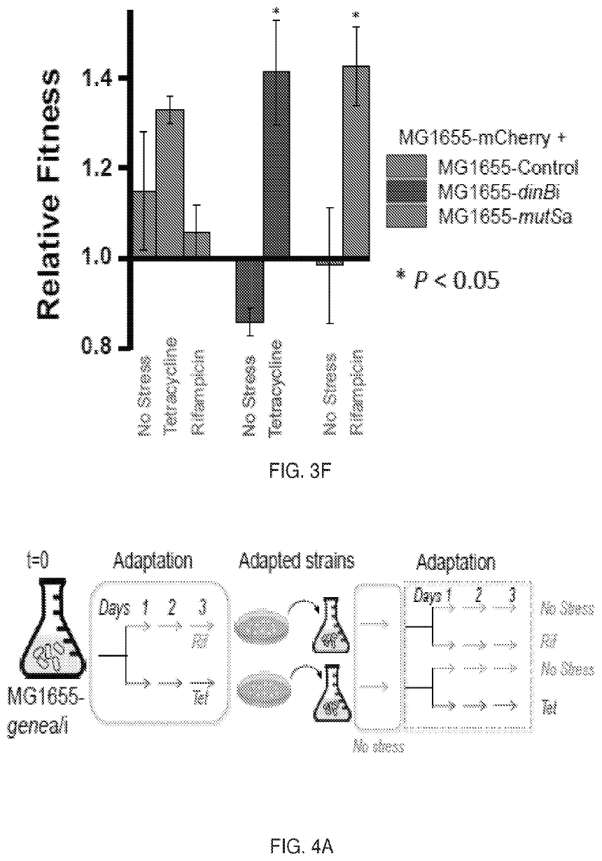

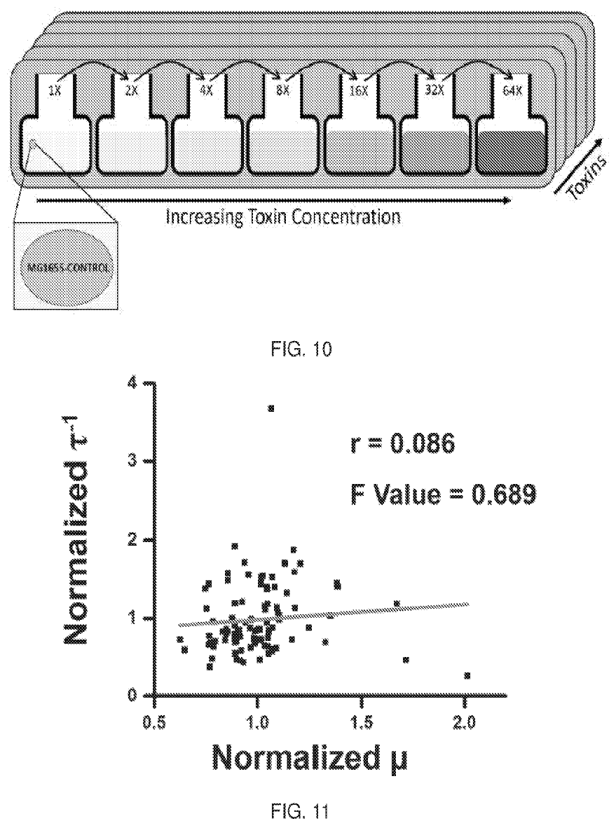

The fitness of strains harboring the CRISPR constructs under various environmental stresses to which infectious bacteria are commonly exposed were examined to determine whether artificial modulation of gene expression enabled control over bacterial fitness (and thus adaptive potential). To achieve this, five stress conditions were chosen, representing oxidizing agents (household bleach and hydrogen peroxide), antibiotics (tetracycline and rifampicin), and nutrient limitation (M9 minimal media supplemented with 0.4% glucose). The Minimum Inhibitory Concentration (MIC) was determined using MG1655-Control to estimate the appropriate starting concentrations for growth under each stress condition (FIG. 10). The MIC of each toxin or nutrient was determined using the MG1655-rfpi strain harboring the RFP-targeting sgRNA control plasmid, along with the dCas9 plasmid induced with 10 ng/μL aTc. A 2-fold serial dilution in concentration was used to test seven concentrations for each toxic compound used in the final adaptation experiments represented in FIG. 2 and FIG. 3. The MIC was determined to be the lowest concentration that prevented a 0.1 increase in OD562 nm after overnight growth in each stress.

The sub-MIC levels were used as starting points for stress exposure experiments (FIG. 2A, see Methods).

E. coli strains harboring the CRISPR constructs were exposed to each stress over a course of 72 hours (FIG. 2B), transferring biological triplicates every 24 hours into fresh media supplemented with aTc and antibiotics to maintain plasmid selection (see Methods). During this time, optical densities were monitored to track changes in growth rate (μ) and inverse lag phase (τ⁻¹) on each day of the experiment (FIG. 2B; Tables 7-9). This data was normalized to MG1655-Control by dividing μ and by the average performance of biological triplicates of MG1655-Control from the experimental day (creating μₙₒᵣₘ and μₙₒᵣₘ). Normalized data was averaged over three experimental days.

Adapting bacterial populations have been demonstrated to exhibit significant heterogeneity in growth rates and lag times, and thus these serve as useful metrics to quantitatively compare adaptive trends between strains. Lag times were kept in their reciprocal format, as larger lag times (smaller inverse lag time, τ⁻¹ₙₒᵣₘ<1.0) indicate that cells are stalling longer before growth and are thus considered detrimental. It was found that the overall correlation between τ⁻¹ₙₒᵣₘ and μₙₒᵣₘ was negligible (Pearson's correlation coefficient, r=0.09, F-value=0.69), indicating that these independently provided insight into changes in growth caused by gene modulation (FIG. 11).

A two-dimensional analysis of normalized τ⁻¹ and μ revealed greater diversity during stress exposure than was observed under no stress (FIG. 2C). FIG. 2C illustrates comparison of μₙₒᵣₘ and τ⁻¹ₙₒᵣₘ averaged over three days, normalized to MG1655-Control. Deviations from intersecting dotted lines (control) indicate impacts on growth characteristics induced by perturbing gene expression with respect to MG1655-Control. The top row illustrates results from inhibition strains, while the bottom illustrates results from activation strains. The y-axis uses Log 2 scaling.

From this data, 17 of the 84 growth rates (14 modulation constructs×6 growth conditions) and 35 of the 84 lag times demonstrated statistically significant shifts from MG1655-Control (FIG. 2D; improvements in growth characteristics (μₙₒᵣₘ, and τ⁻¹ₙₒᵣₘ) are denoted in green; impairments are denoted in red. P-values were calculated using a two-tailed type II t-test. Error bars represent s.d. of biological triplicates). With the exception of MG1655-marAi, none of these shifts occurred in the absence of stress exposure, indicating that modulations of these genes did not inherently diminish or enhance bacterial growth in absence of stress. Calculating the sum of distances (Di and Da for inhibition and activation constructs respectively) from the expected performance under no modulation (μₙₒᵣₘ=τ⁻¹ₙₒᵣₘ=1.0) revealed relatively minor changes under no stress (Di=0.87, Da=0.63) than under the exposure to bleach (Di=2.11, Da=2.46), peroxide (Di=2.84, Da=1.24), glucose limitation (Di=2.60, Da=1.82), and especially the antibiotics rifampicin (Di=2.40, Da=3.12) and tetracycline (Di=2.81, Da=7.70). These results indicate that shifts in growth characteristics from the control strain (deviations from the dotted lines) increased significantly during the presence of stress, demonstrating the potential that synthetically engineered gene modulations have to artificially control the adaptive response.

Performance of modulation strains during exposure to oxidizing agents resulted in reduced growth rates than was observed under other conditions, accounting for four of the six statistically lower μ phenotypes. Under bleach exposure, MG1655-dinBi demonstrated reduced μₙₒᵣₘ (0.76±0.14), P=0.014) and increased τ⁻¹ₙₒᵣₘ (1.44±0.05, P=0.019), while under peroxide exposure MG1655-marAa demonstrated both reduced τ⁻¹ₙₒᵣₘ (0.78±0.04, P=0.033) and τ⁻¹ₙₒᵣₘ (0.48±0.10, P=0.006). Lag times in particular were impacted by gene modulations during exposure to antibiotics, affecting 10 of 14 strains grown in rifampicin and 11 of 14 strains grown in tetracycline. Under rifampicin stress, MG1655-acrAi demonstrated impaired growth characteristics (μₙₒᵣₘ=0.62±0.07, P=0.023 and τ⁻¹ₙₒᵣₘ=0.72±0.05, P=0.008), while MG1655-mutSa presented opposite effects (μₙₒᵣₘ=1.38±0.12, P=0.032 and τ⁻¹ₙₒᵣₘ=1.40±0.16, P=0.011). Under tetracycline stress, both MG1655-recAa and MG1655-marAa demonstrated improved growth rates (μₙₒᵣₘ=1.72±0.26, P=0.0008 and μₙₒᵣₘ=2.01±0.77, P=0.007 respectively) and extended lag times (τ⁻¹ₙₒᵣₘ=0.46±0.12, P=0.001 and τ⁻¹ₙₒᵣₘ=0.26±0.02, P=0.00 respectively). Under glucose limitation, MG1655-soxSi displayed improved growth characteristics (μₙₒᵣₘ=1.09±0.02, P=0.029 and τ⁻¹ₙₒᵣₘ=1.52±0.19, P=0.006), while MG1655-acrAa demonstrated the opposite effect (μₙₒᵣₘ0.65±0.06, P=0.0001 and τ⁻¹ₙₒᵣₘ=0.59±0.19, P=0.017). These results indicate that small artificial modulations in gene expression during stress exposure significantly influence native bacterial adaptive responses.

### Example 3—Competition Assays Confirm Fitness Impact of Gene Modulation

Competition assays between a select subset of MG1655-genei/a strains based on their phenotypic performances under stress, as well as a new control strain “MG1655-mCherry”, to determine whether induced gene modulation provides a competitive advantage (or disadvantage) impacting bacterial fitness. The MG1655-mCherry strain was analogous to MG1655-Control, but also included the coding sequence for mCherry on the sgRNA-RFPi plasmid. By mixing MG1655-mCherry with strains of interest, the relative fitness impacts of gene modulation during stress exposure were determined utilizing flow-activated cell sorting (FACS) (FIG. 3A). A mixture of the two strains grown under stress was analyzed before (D0) and after one day (D1) of stress exposure. The fluorescence of the total population was used to determine the relative ratios of the control strain with basal levels of gene expression (which fluoresced red due to the presence of mCherry) to the strain with perturbed gene expression (which did not fluoresce due to the absence of mCherry). Pure (100%) MG1655-mCherry and MG1655-Control populations distributed into two distinct fluorescence intervals both on D0 and D1 (FIG. 3B). When mixed equally (50% by OD), statistically significant selection for either MG1655-mCherry or MG1655-Control was not observed after one day of exposure to tetracycline or rifampicin when compared to no stress condition (FIG. 3C).

To demonstrate that the MG1655-genei/a strains impacted bacterial fitness during stress exposure, this competition assay approach was used to compare the fitness of MG1655-mutSa under rifampicin and MG1655-dinBi under tetracycline against MG1655-mCherry. These strains were selected for their measured impacts on μ depicted in FIG. 2. Because these strains improved μ in rifampicin or tetracycline, a reduced starting concentration was chosen (30%) relative to MG1655-mCherry (70%) on D0. Activation of mutS and inhibition of dinB caused a shift in the relative population density away from MG1655-mCherry and towards MG1655-mutSa (FIG. 3D) or MG1655-dinBi (FIG. 3E) after one day of exposure to their respective stresses, but not during the absence of stress. These results were reproduced across biological triplicates (FIG. 3F).

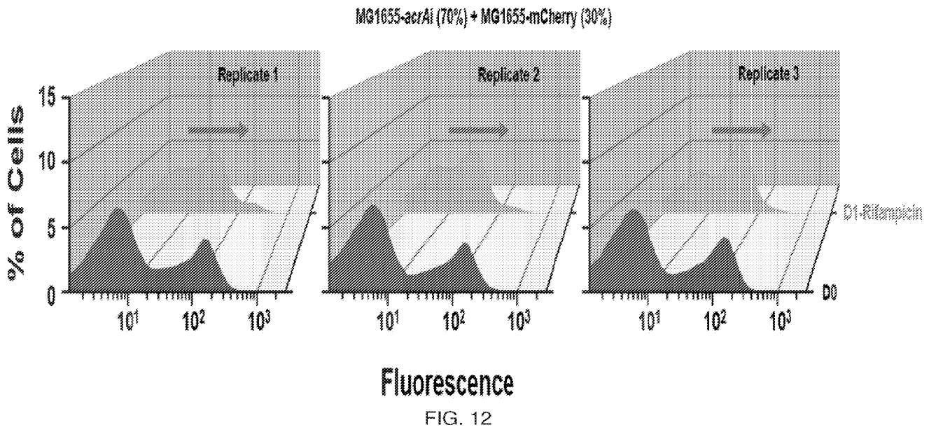

The opposite effect was also observed; when MG1655-acrAi (70%), which exhibited reduced μ under rifampicin stress (FIGS. 2C-2D), was competed against MG1655-mCherry (30%), the latter was selected for despite having a lower starting concentration (30%) when exposed to rifampicin stress (FIG. 12).

This method was utilized to estimate the fitness of each strain relative to MG1655-mCherry (FIG. 3F). FACS data was used to estimate the proportion of red and non-red cells before and after one day of stress exposure, from which Malthusian (m) parameters were calculated for each of the two competing strains. The m ratios were used to calculate relative fitness (see methods) from three biological replicates. The relative fitness of MG1655-Control was not statistically different between antibiotic exposure and no stress exposure conditions. However, the relative fitness of MG1655-dinBi was greater under tetracycline stress (1.41±0.11, P=0.00007), while MG1655-mutSa was greater under rifampicin stress (1.43±0.09, P=0.02) demonstrating that these strains were selected for over MG1655-mCherry only during stress exposure. P-values were calculated using a two-tailed type II t-test, and are in relation to the no stress-condition for each competition experiment. Error bars represent s.d. of biological triplicates.

### Example 4—Phenotypic Reversibility and Evidence of Non-Genetic Impact of Transcriptional Modulation

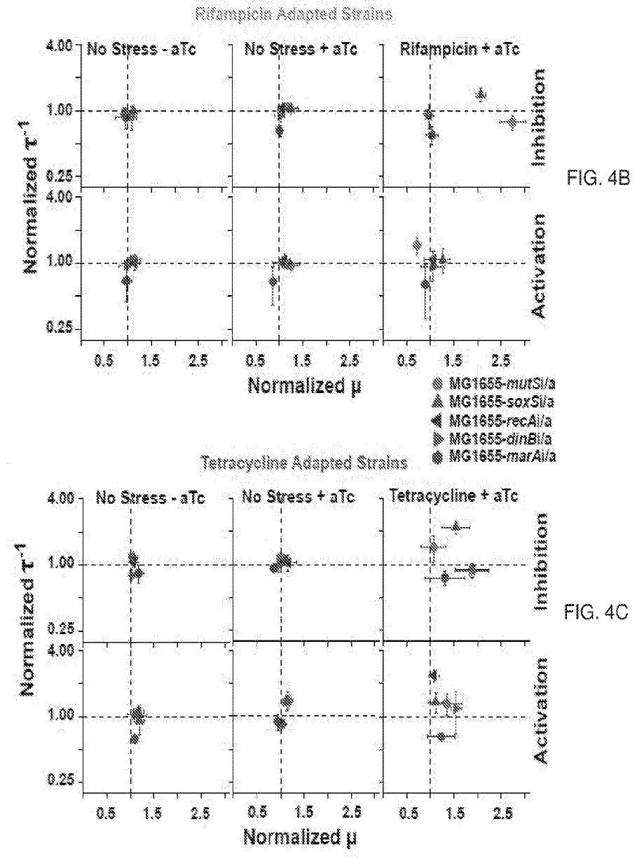

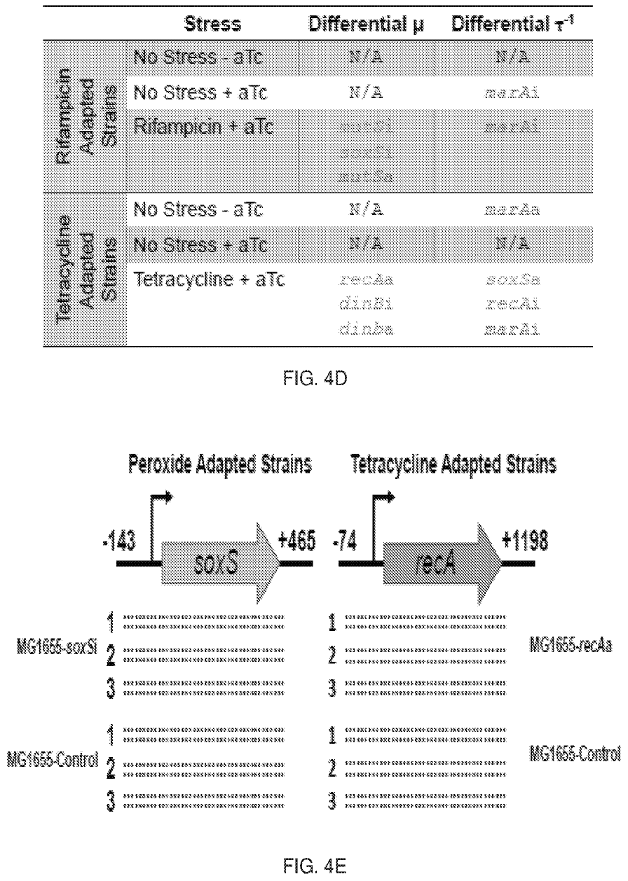

The reversibility of the phenotypic deviations of these experimental strains from MG1655-Control was tested; that is, whether or not the strains demonstrated similar growth characteristics as the control upon removal of stress. Such reversion under no stress is indicative the non-genetic nature of the observed changes in growth characteristics and fitness. Analysis was performed on a subset of biological triplicates collected at the end of three days of exposure to tetracycline and rifampicin stress, as depicted in the schematic of FIG. 4. These strains were grown for one day in the absence of stress and aTc induction. Afterwards, each strain was re-exposed to the three day adaptation experiment in the absence of stress both with and without aTc induction, or the same initial stress and aTc. For rifampicin adapted strains, a return to the wild-type phenotype was observed in no stress both in the absence (Di=0.60, Da=0.96) and presence (Di=0.99, Da=0.75) of aTc induction of gene modulation (FIG. 4B; error bars represent s.d. of biological triplicates). A similar result was observed in tetracycline adapted strains under no stress in the absence (Di=0.90, Da=1.08) and presence (Di=1.24, Da=0.66) of aTc (FIG. 4C). Together, these data indicate that the phenotypic effects of gene expression modulations were reversible upon stress removal, indicating their non-genetic nature.

When stress was maintained, modulation strains continued to demonstrate deviations in μ and τ−1 under rifampicin exposure (Di=3.49, Da=1.38) as well as tetracycline exposure (Di=3.18, Da=3.93). As before, marA modulation increased lag times under no stress alone. A majority of strains exhibited similar phenotypes during both the first and second round of rifampicin exposure. MG1655-marAi again demonstrated a reduced τ⁻¹ₙₒᵣₘ (0.66±0.08, P=0.02). The previously identified lag time impacts of MG1655-recAi, MG1655-recAa and MG1655-dinBa became less pronounced. Two new phenotypes were observed only during the second round of rifampicin exposure: MG1655-mutSi μₙₒᵣₘ (2.74±0.27, P=0.0009) and MG1655-soxSi μₙₒᵣₘ (2.07±0.11, P=0.0004). MG1655-mutSa demonstrated a reduced μₙₒᵣₘ (0.72±0.08, P=0.03), opposite what it had exhibited during the initial round of rifampicin exposure. The difference in phenotypes between the first and second rounds of adaptation can be explained by an altered epigenetic state or accrual of specific mutations over an extended period of adaptation.

Under the second round of tetracycline exposure, no such reversals in phenotype were observed. Six previous statistically significant results remained; MG1655-recAa, MG1655-dinBi and MG1655-dinBa exhibited increased μₙₒᵣₘ (1.89±0.35, P=0.03, 1.55±0.11, P=0.006 and 1.88±0.36, P=0.03), MG1655-soxSa and MG1655-recAi exhibited increased τ⁻¹ₙₒᵣₘ (2.18±0.35, P=0.005 and 2.35±0.08, P=0.0001 respectively), and MG1655-marAi exhibited decreased τ⁻¹ₙₒᵣₘ (0.65±0.03, P=0.01).

The experimental strains were sequenced for mutations. Both MG1655-Control and gene modulation strains received the same basal level of selection pressure to accumulate alterations at the genomic level. Thus, any mutations which may have arisen (or were prevented from arising) were directly influenced by the modulations themselves. However, it is possible that CRISPR modulations could have artificially induced mutations in their genomic targets to circumvent the synthetically induced gene expression changes, undermining the observed phenotypic changes in modulation strains. To test for this possibility, genes directly influenced by modulation in a subset of strains were sequenced (FIG. 4E). The region of soxS in MG1655-soxSi was sequenced after exposure to peroxide stress, as well as the region of recA in MG1655-recAa after exposure to tetracycline stress, since these gene modulations had a significant impact on μ and τ⁻¹ respectively. MG1655-Control was also sequenced after exposure to each condition, to account for any mutations not influenced by gene modulation. Sequencing of six biological replicates per strain provided no evidence of mutations, indicating that these modulations did not induce mutations in these genomic regions.

### Example 5—Design and Characterization of Strains Having Multiple Targets Perturbed Simultaneously

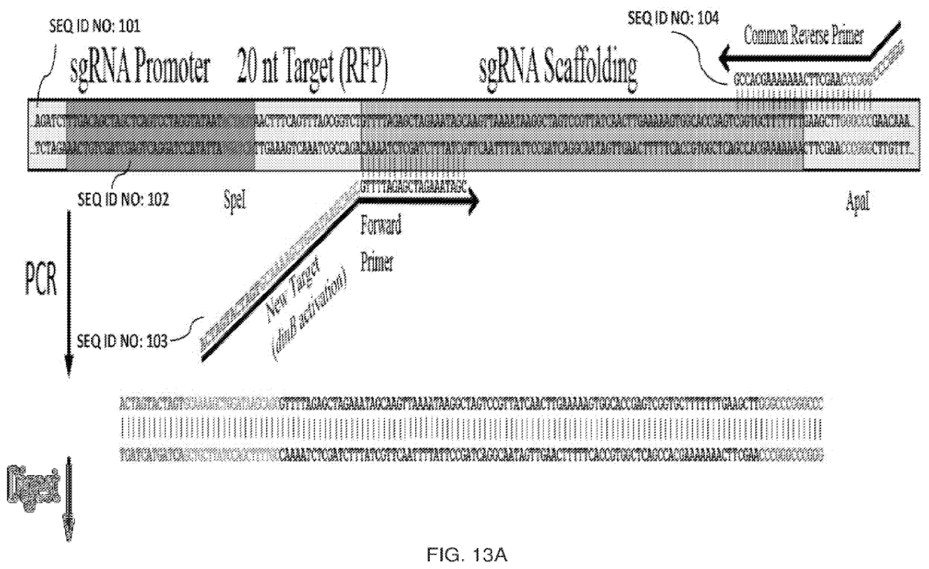

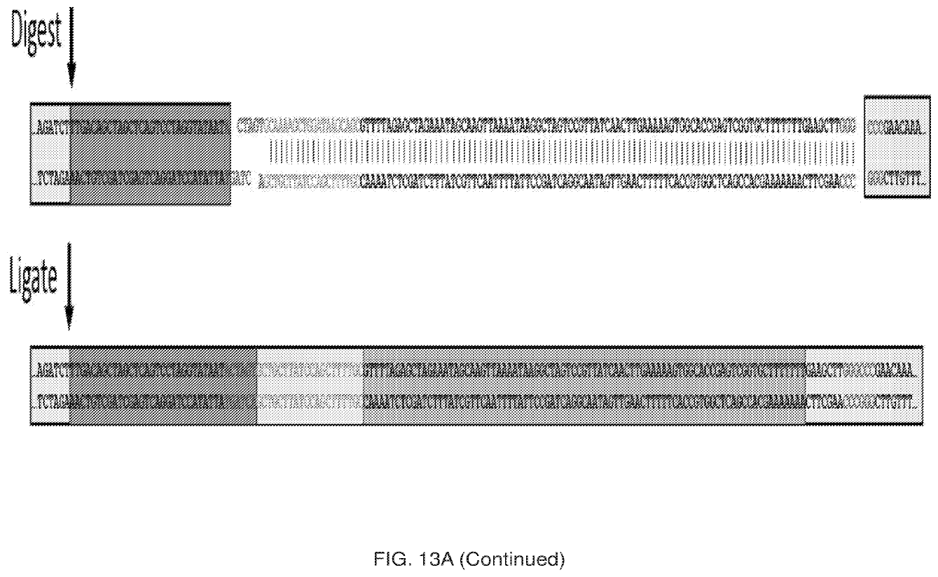

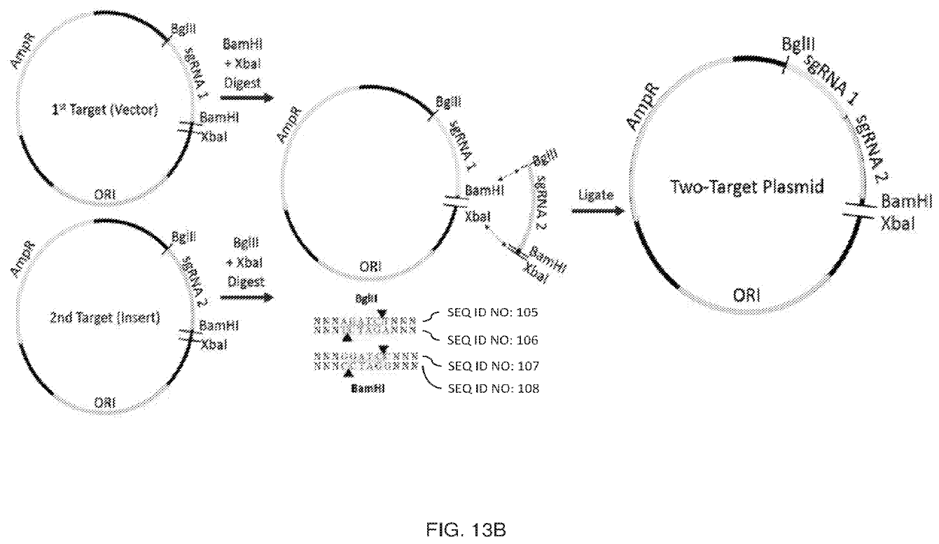

The combinatorial effects of regulating multiple genes simultaneously were explored. Combining several independent sgRNA targets into one construct could allow for controlled modulation of multiple genes' expression patterns in a more specific fashion than could be achieved through contemporary methods such as global transcription machinery engineering. Simultaneous induction of synthetic gene modulations was achieved using a modified cloning approach that introduced tandem independent sgRNAs onto one plasmid to combine in vivo with dCas9 or dCas9-ω (FIG. 13). Three constructs were designed in which the perturbed genes had known regulatory interactions: MG1655-tolCi-acrAi (which inhibited the AcrAB-TolC multidrug efflux pump), MG1655-mutSa-dinBi (which activated expression of a mismatch-repair protein and decreased expression of an error-prone polymerase, thereby likely decreasing mutation rates), and MG1655-recAa-dinBa (which increased expression of dinB both directly and indirectly by increasing expression of its upstream up-regulator recA). A fourth construct, MG1655-tolCi-acrAi-soxSi, which inhibited three genes simultaneously which displayed similar impacts on growth characteristics in FIGS. 2C-2D (tolCi and soxSi increased μ under glucose limitation and τ under rifampicin exposure, while acrAi and soxSi increased μ under tetracycline exposure). A fifth construct was designed, MG1655-tolCi-dinBi, which perturbed two genes in separately regulated pathways and have not been demonstrated to produce similar impacts on growth characteristics under the same stress condition.

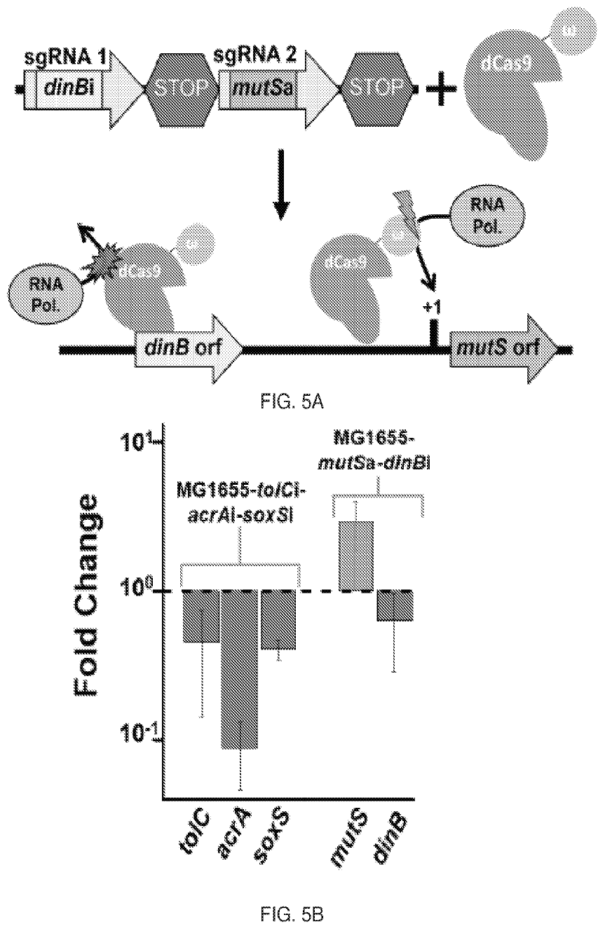

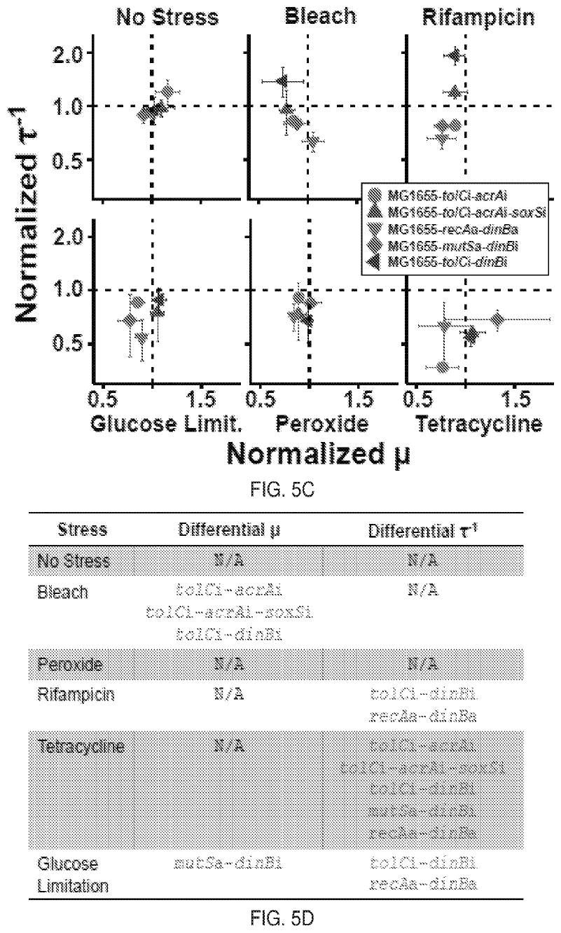

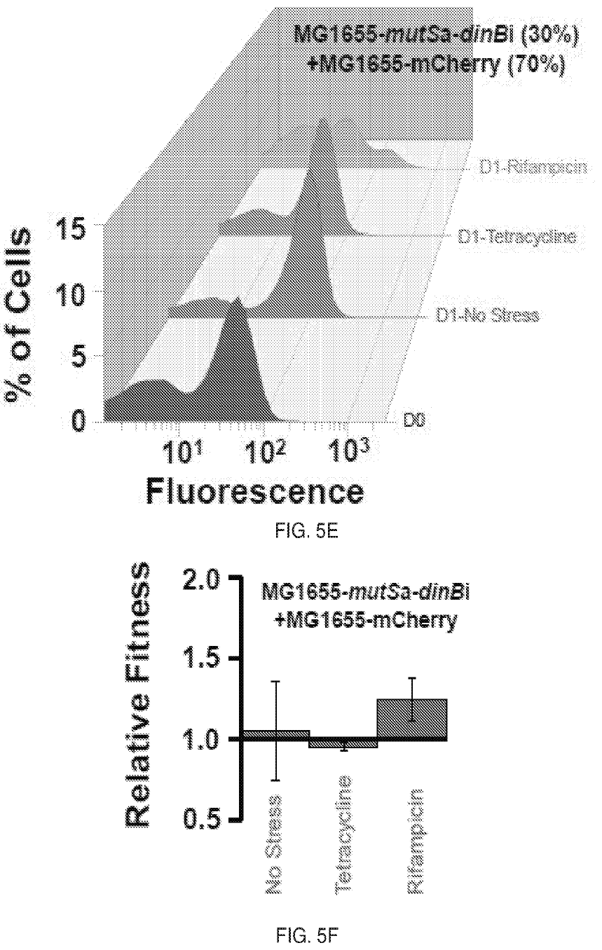

Strains engineered to only exhibit inhibited gene expression utilized dCas9, while strains engineered to exhibit activation of one or more genes utilized dCas9-w. Strain MG1655-mutSa-dinBi demonstrates that simultaneous activation and inhibition of gene expression is possible through the use of dCas9-ω in a fashion similar to that reported in yeast (FIG. 5A). RT-qPCR was used to verify that MG1655-mutSa-dinBi (as well as MG1655-tolCi-acrAi-soxSi) perturbed gene expression as intended (FIG. 5B). Growth for 72-hours under stress was repeated for strains harboring these multi-target synthetic constructs.

The effects of modulating gene expression on growth characteristics were quantified, again normalizing data against the MG1655-Control strain grown alongside the multi-target strains (FIG. 5C and Tables 7-10). 4 of 30 measured μ (5 modulation constructs×6 growth conditions×2 growth characteristics) and 9 of 30 measured τ−1 were significantly impacted by simultaneous gene modulations. Surprisingly, 11 of the 13 statistically different growth characteristics measured by these strains were detrimental, i.e. decreased μₙₒᵣₘ or τ-lnorm. The only two improvements were exhibited by a higher τ⁻¹ₙₒᵣₘ of MG1655-tolCi-dinBi under rifampicin stress and glucose limitation (FIG. 5D). Conversely, MG1655-recAa-dinBa demonstrated reduced τ⁻¹ₙₒᵣₘ under three different conditions: rifampicin stress (τ⁻¹ₙₒᵣₘ=0.66±0.09, P=0.02), tetracycline stress (τ⁻¹ₙₒᵣₘ=0.63±0.22, P=0.01) and glucose limitation (τ⁻¹ₙₒᵣₘ=0.54±0.14, P=0.003). The modulation of recAa and dinBa alone had reciprocal impacts under rifampicin and tetracycline stress, and no significant impact under glucose limitation. These results indicated that the growth of strains with simultaneously perturbed genes was diminished in relation to single target strains.

As before with single-gene modulation, competition assays between MG1655-mutSa-dinBi and MG1655-mCherrry were performed to further analyze the fitness impacts induced by simultaneous gene modulation. No selection was observed for MG1655-mutSa-dinBi after one day of stress exposure in either rifampicin or tetracycline (FIG. 5E). Furthermore, using Malthusian (m) parameters calculated from biological triplicates, there was no statistically significant difference between the relative fitness of MG1655-mutSa-dinBi under no stress and the relative fitness under either stress (FIG. 5F). This data contrasts the improved fitness of MG1655-mutSa and MG1655-dinBi in rifampicin and tetracycline respectively that was previously observed (FIG. 3D-F). This indicates that strains in which multiple genes are perturbed are less fit than would be expected based on results from single-gene modulation strains.

### Example 6—Simultaneous Gene Modulation Predominantly Yields Negative Epistatic Interactions

The epistasis induced by simultaneous gene modulation (recently characterized as ‘epigenetic’ epistatic interactions) was then examined, given that a large number of strains harboring multiple gene targeting constructs elicited a less-fit phenotype than predicted from the performance of strains harboring their single-target constituents. To do so, a multiplicative model was used to calculate epistasis in both μₙₒᵣₘ and τ⁻¹ₙₒᵣₘ for each of the five strains with simultaneous gene modulations. The expected growth rates (or inverse lag times) of these strains were calculated by multiplying together the μₙₒᵣₘ (or τ-lnorm) of each single gene modulation strain from which they were created, and epistasis was calculated as the difference between these expected values and those that were measured (see Methods).

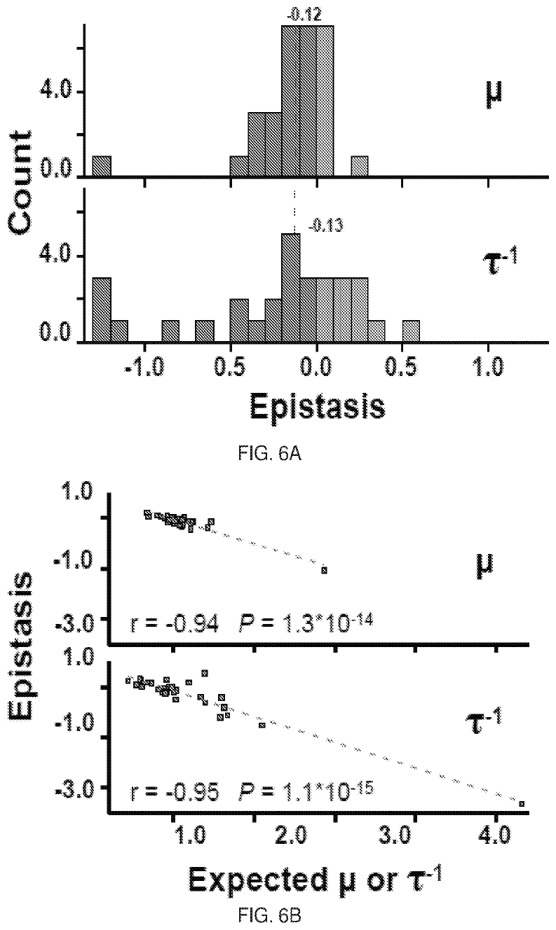

Epistasis in μₙₒᵣₘ and τ⁻¹ₙₒᵣₘ was quantified for each strain under each growth condition, and the distribution of epistasis was analyzed (FIG. 6A). A distinct pervasiveness of negative epistasis resulting from simultaneous gene modulation in both μₙₒᵣₘ (mean epistasis=−0.17±0.14 [95% confidence interval], P=0.02) and in τ⁻¹ₙₒᵣₘ (mean epistasis=−0.35±0.33 [95% confidence interval], P=0.04) was observed. A large majority of the data (73% of μₙₒᵣₘ epistasis and 63% of τ⁻¹ₙₒᵣₘ epistasis) falls into the category of negative epistasis, and both distributions are markedly skewed towards greater magnitudes of negative epistasis. The data indicate that when two or more genes are perturbed from their basal expression levels, their combinatorial fitness benefits are abated or their disadvantages amplified.

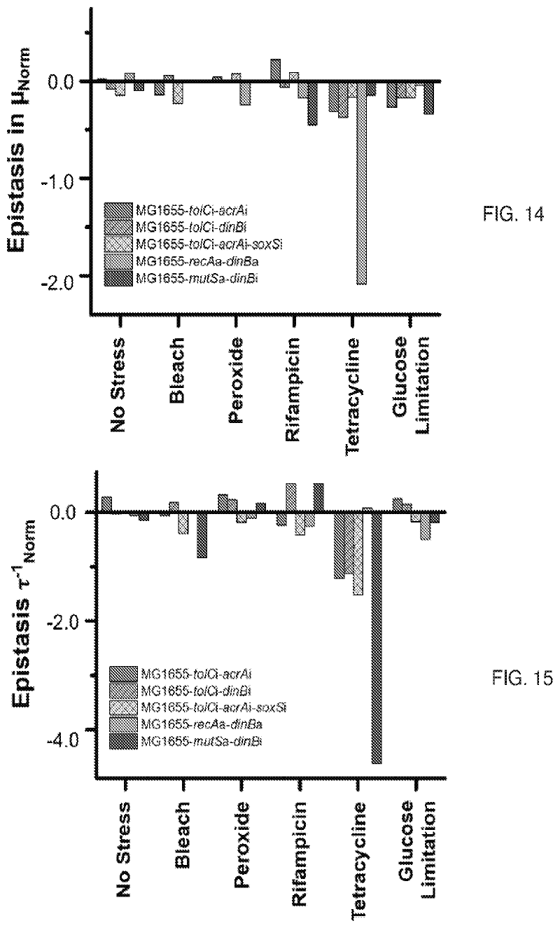

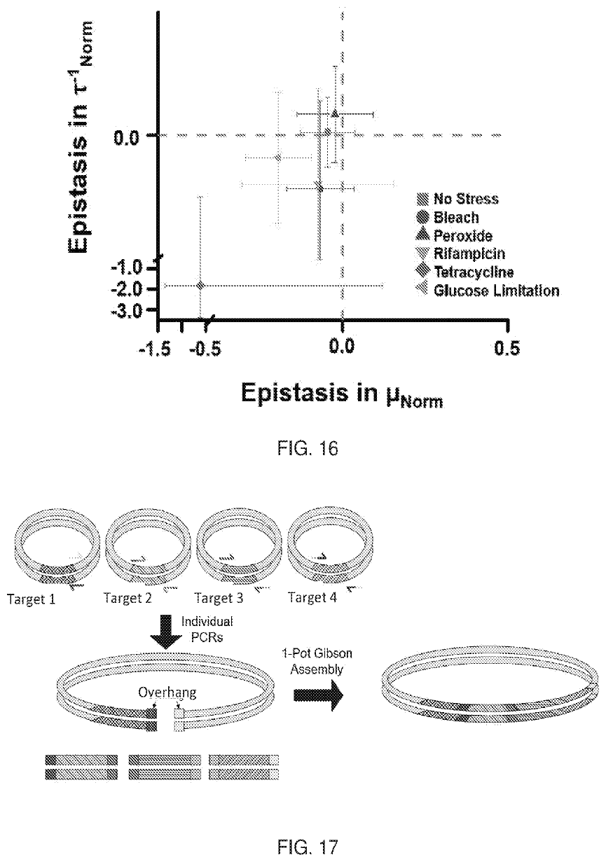

An analysis of epistasis in μₙₒᵣₘ (FIG. 14) and τ⁻¹ₙₒᵣₘ (FIG. 15) of each strain indicated that the magnitude of epistasis depended more heavily on the stress exposure, rather than the strain itself. Clustering analysis revealed that epistatic trends group by stress (FIG. 16). This was apparent especially under tetracycline exposure, which resulted in much larger degrees of negative epistasis in both μₙₒᵣₘ and τ-lnorm. These findings indicate disruption of stress-dependent adaptive pathways. Strong negative correlations between expected growth characteristics and their measured epistasis were also observed (FIG. 6B). Removing the most negative value of epistasis still resulted in significant negative correlations (r=−0.70, P=2.4*10-5 and r=−0.79, P=1.1*10-7 for μₙₒᵣₘ−1 norm respectively). This illustrates that negative epistatic effects are strongest whenever multiple gene modulations, which individually prove beneficial, are subsequently combined. This trend resembles diminishing returns epistasis, wherein the fitness gains of consecutive mutations decelerate during adaptation. This phenomenon has been reproduced across a number of studies, and has been correlated to mutations which specifically impact gene expression. The data presented here demonstrate that an inherent fitness cost is associated with excessive modulations of gene expression levels, and that epigenetic interactions can be subjected to the same diminishing returns epistasis typically associated with mutations.

### Example 7—Materials and Methods for Examples 1-6

### Bacterial Strains, Media and Culture Conditions.

E. coli cloning strains NEB 10-β (New England Biolabs) and DH5α (Zymo Research Corporation), as well as the final experimental strain K-12 MG1 (ATCC 700926) were cultured in Luria-Bertani Broth (LB) (Sigma-Aldrich®) unless otherwise noted. Colonies were grown on LB-agar plates supplemented with ampicillin (100 μg/mL) and chloramphenicol (25 μg/mL). For nutrient limiting conditions and growth of RT-qPCR biological triplicates, M9 minimal media (5×M9 minimal media salts solution from MP Biomedicals, 2.0 mM MgSO4, and 0.1 mM CaCl₂ in sterile water) was used in lieu of LB, supplemented with 0.4% vol/vol glucose (34.2 mM). Expression of dCas9 and dCas9-ω was induced from plasmids pdCas9 and pPO-das9ω respectively by adding aTc (10 ng/mL) to the media. During competition experiments, aTc concentration was increased to 25 ng/mL to assist in distinguishing fluorescent populations from non-fluorescent ones. Cloning strains were made chemically competent with the Mix & Go E. coli Transformation Kit & Buffer Set (Zymo Research Corporation), and the final sgRNA plasmids were transformed into electrocompotent MG1655 cells harboring either pdCas9 or pPO-das9ω for inhibition or activation of gene expression respectively. The final experimental strains are listed in Table 4. Cultures (2-5 mL) were grown at 37° C. with constant shaking at 225 r.p.m. All experiments used biological triplicates inoculated from individual colonies grown on LB-agar plates supplemented with ampicillin and chloramphenicol.

### Plasmid Assembly.

A list of plasmids used in this study is provided in Table 4. dCas9-ω from pWJ66 (Addgene plasmid 46570) was first cloned into the same vector as dCas9 from pdCas9 (Addgene plasmid 44249) under the same aTc inducible promoter, rrnB T1 terminator and chloramphenicol resistance marker to create plasmid pPO-dCas9ω. Single-target sgRNA plasmids were first derived from the RFP-targeting control sgRNA-RFPi (Addgene plasmid 44251) using the approach outlined in FIG. 13. A unique forward primer coding for the new 20 nt target, as well as overalap with the gRNA scaffolding, was used along with a common reverse primer to create new gRNA target inserts using PCR. These PCR products, along with the gRNA plasmid, were digested and ligated to form new gRNAs. Sequencing was used to confirm each sgRNA target after construction. As depicted in FIG. 13B, the unique sgRNA target “2” was cloned into the plasmid containing sgRNA target “1.” Both plasmids were digested with XbaI, while the first and second plasmids were digested with BamHI and BglI, respectively. Due to the compatible sticky ends that are generate, the second sgRNA can be ligated into the first plasmid without regenerating a restriction site. This process can be repeated to create sgRNA plasmids targeting “n” number of gens.

Primers were designed to replace the 44251 plasmid's RFP-targeting sgRNA using a common reverse primer (Reverse sgRNA) flanked with an ApaI restriction site and unique forward primers flanked with a SpeI restriction site, listed in Table 5. PCR with Phusion® High-Fidelity DNA Polymerase (New England Biolabs) was used to amplify these new target sgRNA-insert DNA fragments, which were subsequently gel-purified (Zymoclean™ Gel DNA Recovery Kit-Zymo Research Corporation), digested with ApaI and SpeI (FastDigest Enzymes-Thermo Scientific) as per the provided protocols and PCR-purified (GeneJET PCR Purification Kit-Thermo Scientific). The 44251 plasmid (Addgene) backbone was also digested with ApaI and SpeI and gel purified, and T4 DNA Ligase (Thermo Scientific) was used to ligate the new sgRNA target inserts into the 44251 backbone. Ligations were transformed into chemically competent DH5α or NEB 10-β cells. Plasmids minipreps were performed using Zyppy™ Plasmid Miniprep Kit (Zymo Research Corporation). Sequencing of final sgRNA constructs were performed for validation of correct assembly product (GENEWIZ).

To create the fluorescent control for competition assays, mCherry from pHL662 (Addgene plasmid 37636) was PCR amplified with AatII restriction sites on either end of the resulting fragment, and cloned onto sgRNA-RFPi under an aTc inducible promoter. This construct was transformed into MG1 harboring pdCas9 to create MG1655-mCherry. To create multi-gene targeting sgRNA plasmids, the above single gene targeting sgRNAs were combined using the procedure outlined in FIG. 13B. The first sgRNA target plasmid was digested with BamHI and XbaI and the 2569 bp vector was gel-purified. A second target plasmid was digested using BgIII and XbaI and the 527 bp insert was gel-purified. These were ligated and transformed into DH5α chemically competent cells and plated on LB-ampicillin plates. BamHI and BgIII digestion overhangs produce compatible sticky ends that, when ligated together, do not produce a new restriction enzyme site. After recovering these plasmids, a BamHI digestion was used to confirm the plasmids were the correct size on an agarose gel. To create more than two targets, the same approach was applied using the BamHI/XbaI digestion on the two gene targeting plasmid and the BgIII/XbaI digestion on the third target. All inserted fragments were confirmed by sequencing.

TABLE 5
Cloning and sequencing primers used. Bolded,
underlined sequence indicates target sequence.

| | | SEQ ID |
|---|---|---|
| Name | Sequence | NO: |
| Forward mutS-a | ACTAGTACTAGTGCAAGTACGC | 19 |
| | AAAATTGTATGTTTTAGAGCTA | |
| | GAAATAGC | |
| Forward mutS-i | ACTAGTACTAGTCTGCTGCATC | 20 |
| | ATGGGCGTATGTTTTAGAGCTA | |
| | GAAATAGC | |
| Forward soxS-a | ACTAGTACTAGTGCGTTTCGCC | 21 |
| | ACTTCGCCGGGTTTTAGAGCTA | |
| | GAAATAGC | |
| Forward soxS-i | ACTAGTACTAGTCTACATCAAT | 22 |
| | GTTAAGCGGCGTTTTAGAGCTA | |
| | GAAATAGC | |
| Forward tolC-a | ACTAGTACTAGTAGCAGTCATG | 23 |
| | TGTTAAATTGGTTTTAGAGCTA | |
| | GAAATAGC | |
| Forward tolC-i | ACTAGTACTAGTGGCTCAGGCC | 24 |
| | GATAAGAATGGTTTTAGAGCTA | |
| | GAAATAGC | |
| Forward acrA-a | ACTAGTACTAGTGAGCCACATC | 25 |
| | GAGGATGTGTGTTTTAGAGCTA | |
| | GAAATAGC | |
| Forward acrA-i | ACTAGTACTAGTAGCATCAGAA | 26 |
| | CGACCGCCAGGTTTTAGAGCTA | |
| | GAAATAGC | |
| Forward recA-a | ACTAGTACTAGTCCGTGATGCG | 27 |
| | GTGCGTCGTCGTTTTAGAGCTA | |
| | GAAATAGC | |
| Forward recA-i | ACTAGTACTAGTTACCAAATTG | 28 |
| | TTTCTCAATCGTTTTAGAGCTA | |
| | GAAATAGC | |
| Forward dmB-a | ACTAGTACTAGTGCAAAAGCTG | 29 |
| | GATAAGCAGCGTTTTAGAGCTA | |
| | GAAATAGC | |
| Forward dmB-i | ACTAGTACTAGTATTGTCGCGC | 30 |
| | ATCTCCACTGGTTTTAGAGCTA | |
| | GAAATAGC | |
| Forward marA-a | ACTAGTACTAGTGTTTTGTTCA | 31 |
| | ATGCGATGCAGTTTTAGAGCTA | |
| | GAAATAGC | |
| Forward marA-i | ACTAGTACTAGTCCAGTCCAAA | 32 |
| | ATGCTATGAAGTTTTAGAGCTA | |
| | GAAATAGC | |
| Forward dCas9-ω | AGATCTAGATCTAAAGAGGAGA | 33 |
| | AAGGATCTATGGATAAGAAATA | |
| | CTCAAT | |
| Reverse dCas9-ω | CTCGAGCTCGAGTTAACGACGA | 34 |
| | CCTTCAGCAA | |
| sgRNA | GGGGGGGACGTCTAAGAAACCA | 35 |
| sequencing | TTATTATCATG | |
| Reverse sgRNA | GGGCCCGGGCCCAAGCTTCAAA | 36 |
| | AAAAGCACCG | |
| Forward soxS | CTATTGCCAGGGATGGTTC | 37 |
| sequencing | | |
| Reverse soxS | TTTCATAGAAATGCAGCGCC | 38 |
| sequencing | | |
| Forward recA | GGATGTTGATTCTGTCATGG | 39 |
| sequencing | | |
| Reverse recA | TATGCATTGCAGACCTTGTG | 40 |
| sequencing 1 | | |
| Reverse recA | AGTAGACGTTATCGTCGTTG | 41 |
| sequencing 2 | | |

### RT-qPCR Analysis.

Biological triplicate cultures inoculated from individual colonies were grown with appropriate antibiotics overnight in 2.5 mL M9 minimal media. Cultures were induced with aTc the following morning for three hours, and cell pellets were subsequently collected and RNA extracted using the GeneJET RNA Purification Kit (Thermo Scientific) supplemented with lysozyme and proteinase K. Collected RNA was then purified using the TURBO DNA-free kit (Ambion) for heavy DNA contamination. cDNA was synthesized from these RNA samples using 10 μL reactions of the DyNAmo SYBR Green 2-Step qRT-PCR kit (Thermo Scientific). A control reverse-transcriptase-free reaction was included in tandem with all cDNA synthesis reactions. Technical duplicates of RT-qPCR reactions were performed in 10 μL reactions using 2 ng of cDNA and 0.5 μM primers listed in Table 6. Primer efficiency and specificity were previously confirmed. Samples were run on an Eco Real-Time PCR System (Illumina) in the CU Core Sequencing Facility operating the Eco Software v4.1.2.0. RT-qPCR reactions of neighboring genes' expression were performed in 20 μL using 2 ng of cDNA and 0.5 μM primers, and were run on a QuantStudio™ 6 Flex Real-Time PCR System (Thermo Scientific) in the CU Core Sequencing Facility. An initial 10 min polymerase activation at 95° C. was performed, followed by 40 cycles of 95° C. 15 second denaturation, 55° C. 30 second annealing, and 72° C. 30 second extension. Rox normalization was used to compare qPCR samples, and the average Cq values of technical duplicates were used to calculate the ΔΔCq values using rrsA gene expression as a housekeeping gene, which was also averaged. Fold change was calculated using 2-ΔΔCq 2-ΔΔCq values for individual biological triplicates, which were subsequently used to obtain averages and standard deviations.

### MIC Determination.

MICs were first determined via overnight growths of MG1655 harboring dCas9 and RFP-targeting sgRNA with no induction, and measuring the change in OD at 562 nm. A range of concentrations for the disinfectants (hydrogen peroxide and bleach) and the antibiotics (rifampicin and tetracycline) were tested, reducing the concentration by half between. MIC tests started at concentrations of 2% vol/vol, 1.25% vol/vol, 10 ng/μL and 100 ng/μL for bleach, hydrogen peroxide, rifampicin and tetracycline respectively. MICs were determined to be the lowest concentration which prevented a change of 0.1 OD between days. The final sub-MIC values used in this experiment, as well as a description of mechanisms of action, are presented FIG. 2A.

Stress conditions were selected to monitor a broad range of antimicrobials. Peroxide and bleach introduce oxidative stress by producing highly reactive superoxide or chlorine radicals respectively. Tetracycline inhibits protein synthesis by disrupting tRNA interactions with the ribosome, while rifampicin inhibits transcription by preventing the activity of RNA polymerase. These antibiotics avoided mechanistic overlap with the antibiotics required to maintain plasmid selection, ampicillin and chloramphenicol, which inhibit cell-wall-synthesis and peptide bond formation respectively.

TABLE 6
RT-qPCR primers used.

| | | SEQ ID |
|---|---|---|
| Primer Name | Sequence | NO: |
| mutS | ATGGAACGTGAGCAGGACAG | 42 |
| forward | | |
| mutS | CAGCCAGCGTTTCAGCATAC | 43 |
| reverse | | |
| soxS | TCTGCTGCGAGACATAACCC | 44 |
| forward | | |
| soxS | ACTTGCAACGAATGTTCCGC | 45 |
| reverse | | |
| tolC | ACGCACTACCACCAGTAACG | 46 |
| forward | | |
| tolC | TTTGTCTTCCGGGACCAGTG | 47 |
| reverse | | |
| acrA | AAGCCCTTCTTCCAGACGTG | 48 |
| forward | | |
| acrA | AACGGCAAAGCCAAAGTGTC | 49 |
| reverse | | |
| recA | ATCGCCTGGCTCATCATACG | 50 |
| forward | | |
| recA | GCACTGGAAATCTGTGACGC | 51 |
| reverse | | |
| dinB | GGCCAGTTTGTGATTACGCC | 52 |
| forward | | |
| dinB | CTACGCTCCCACAAAATGCG | 53 |
| reverse | | |
| marA | AATCGCGCAAAAGCTGAAGG | 54 |
| forward | | |
| marA | GCGATTCGCCCTGCATATTG | 55 |
| reverse | | |
| rrsA | AACACATGCAAGTCGAACGG | 56 |
| forward | | |
| rrsA | AATCCCATCTGGGCACATCC | 57 |
| reverse | | |
| nudF | CGCAGTCTTGCTACCCTTTG | 58 |
| forward | | |
| nudF | ACGGGCAACATCTTCCACAC | 59 |
| reverse | | |
| ygbA | GCAAGCGTATCTCTCGTGAA | 60 |
| forward | | |
| ygbA | CGCCGAACACACATTTATCC | 61 |
| reverse | | |
| lafU | TCGGACGCACTTTTGGTCAG | 62 |
| forward | | |
| lafU | CAACTGGAACCTTTCGGGTG | 63 |
| reverse | | |
| yafN | TGATCAACCGGTTGCGGTTC | 64 |
| forward | | |
| yafN | ATCTTGCAGCACTTGGACGG | 65 |
| reverse | | |
| ygiB | AATACGCCACCCGTGAAGAC | 66 |
| forward | | |
| ygiB | GACGCCCCATCATGTAACCG | 67 |
| reverse | | |
| pphB | GGAGAGCGAATTACTCTGGC | 68 |
| forward | | |
| pphB | GGTTAGCGAACGTCTGAATG | 69 |
| reverse | | |

### Stress Growth Conditions.

Biological triplicates were inoculated from individual colonies of MG1 cultures harboring both dCas9/dCas9-ω and sgRNA plasmids into 100 μL LB cultures supplemented with ampicillin, tetracycline and aTc and grown overnight to stationary phase. The next day, 2 μL was used to inoculate one 50 μL M9 culture and five 50 μL LB cultures in a 384-well microplate, all of which were supplemented with aTc and the appropriate antibiotics. Four of the LB cultures were supplemented with either 37.3 mM sodium hypochlorite (BLEACH-RITE®, Current Technologies), 0.3 mM hydrogen peroxide (Macron Fine Chemicals), 1.0 ng/μL tetracycline (Sigma-Aldrich®), or 10.0 ng/μL rifampicin (Sigma-Aldrich®) respectively. Bleach stress was increased to 74.6 mM and 149.2 mM on days two and three respectively, and peroxide stress was increased to 0.6 mM on day two and three to maintain selection pressure. Cultures for subsequent experimental days were created as described above and inoculated with 2 μL of the previous day's culture. Culture growth was monitored in 384 microplate wells in a GENios plate reader (Tecan Group Ltd.) operating under the Magellan™ software (version 7.2). Optical densities were measured at 562 nm absorbance in 20 minute intervals. Temperature was maintained at 37° C., and cultures were shaken for 16.6 minutes after each measurement with an additional 10 seconds of shaking before measurement. Data output was used to construct raw growth curves over multiple days (FIG. 9), and growth rates (μ) and lag times (τ) were determined using the GrowthRates version 1.869 and calculated for each day (Tables 7-9). This program estimates the period of exponential growth and excludes lag and stationary phases from calculation of μ. Data were normalized to the performance of strain MG1655-Control over the course of three days. After normalization, lag times were inverted to simplify analysis for the reader. As larger lag times are considered detrimental, inverting these values made values below 1.0 appear detrimental, as they are for growth rates. Distance from μₙₒᵣₘ=1.0, τ⁻ₙₒᵣₘ=1.0 on each graph was calculated as D=Σn\sqrt{(μₙ−1)²+(τ⁻¹ₙ−1)²}, where n is each of the seven inhibition or activation modulations strains from their respective graphs. At the end of adaptation experiments, glycerol stocks of cultures were saved. For re-adaptation experiments, glycerol stocks of the original biological triplicates were streaked onto LB agar plates containing antibiotics and grown overnight. Individual colonies were used to inoculate LB cultures containing antibiotics and grown for 16 hours. Afterwards, cultures were diluted 1/10 into fresh LB containing antibiotics and grown for 24 hours. From this point, the protocol for the original three-day adaptation experiment was performed for cultures under no-stress, no-stress and aTc induction, and the original stress condition (rifampicin or tetracycline) and aTc induction.

### Competitive Fitness Assays.

Gene modulation strains MG1655-mutSa, MG1655-dinBi, MG1655-acrAi and MG1655-mutSa-dinBi, as well as the control strain MG1655-Control, were competed against MG1655-mCherry. mCherry fluorescence measured at 610 nm was used to distinguish the two populations during competition experiments using FACS. To measure the fitness of experimental strains relative to MG1655-mCherry, 2 mL LB cultures supplemented with ampicillin, chloramphenicol and aTc were inoculated from individual colonies and grown overnight for 16 hours. The cultures were then diluted 1:10 in supplemented LB and grown for two hours. Culture ODs (at 562 nm) were then measured and used to mix the two cultures together at either 1:1 or 3:7 OD ratio as indicated in the figures and text. A total of 200 μL of cultures were mixed, out of which 10 μL was added to 190 μL of each stress or non-stress condition per biological replicate. The remaining volume was used for FACS analysis of pre-experiment starting population distributions (D0). Cultures were grown in 96 well microplates in the GENios plate reader as described previously for one day, and the final cultures were collected for FACS analysis.

Relative fitness was determined using the ratio of Malthusian parameters (m) of each experimental strain against m of competitor strain MG1655-mCherry. Malthusians were calculated as m=ln (Nf/Ni) where Nf and Ni are the number of final and initial cells in each mixture respectively. Initial and final cell counts were determined from FACS analysis, and adjusted to represent their respective dilutions.

### Flow Cytometry.

Samples for FACS analysis were washed twice in phosphate-buffered saline (PBS) and resuspended in PBS+4.0% para-formaldehyde (Fisher Scientific). Samples were diluted 1:10 in PBS and sorted using a CyAn ADP analyzer cytometer. Samples were kept on ice throughout the procedure. From each sample 100,000 cells were counted using a voltage of 920 V in a PE-Texas Red channel, a forward scatter gain of 40, and a side scatter voltage of 550 V for detection of mCherry fluorescence. Cells which fluoresced above an intensity of 20 were determined to be MG1655-mCherry, while those below 20 were determined to be the experimental strains. FACS data was analyzed using Matlab and Summit software.

Sequencing of experimental strains. Glycerol stocks of cultures saved at the end of three days of stress exposure were streaked onto LB agar plates containing antibiotics. Two colonies from each biological replicate were picked and used to perform colony PCR using Phusion high-fidelity DNA polymerase. Primers used for these reactions are listed in Table 5, and resulted in the fragments listed. PCR products were subsequently gel-purified. Sequencing reactions were performed using each reverse primer by GENEWIZ. Sequences were aligned to E. coli MG1655 NCBI reference genome NC_000913 using NCBI BLASTN.

### Determination of Epistasis.

Expected growth rates were calculated assuming a multiplicative model of deviations from the relative control values: for example, μ_{Expected,gene1,2}μ_{gene}1*μ_{gene}2, where μ_{gene}1 represents the growth rate observed in the strain targeting gene “1” individually. This provided the expected shift in growth rates (with respect to MG1655-Control) of the multi-gene targeting strains based on results from their individual gene targeting strains. Epistasis was calculated as the difference between the observed relative growth rate (μ_{observed}) of the multi-target strains and this calculated expected growth rate (Epistasis=μ_{observed}−μ_{expected}). Calculations of epistasis in inverse lag times were analogous to this procedure. Epistasis was calculated for all five strains with simultaneously perturbed gene expression under all six growth conditions based on the average values presented in Table 10. The 95% confidence interval for average fitness epistasis was calculated using standard error. A z-test was performed to calculate the probability that this deviated from the null hypothesis of no epistasis, and the resulting P-value was obtained assuming a two-tailed distribution.

TABLE 7
Day 1 calculations for average normalized growth rate and normalized inverse lag

| | Average Normalized | StDev Normalized | Average Normalized | StDev Normalized |
|---|---|---|---|---|
| | Growth Rate | Growth Rate | Inverse Lag Time | Inverse Lag Time |
| No Stress | | | | |
| MG1655-mutSi | 1.11 | 0.13 | 1.23 | 0.31 |
| MG1655-soxSi | 1.18 | 0.24 | 1.03 | 0.09 |
| MG1655-tolCi | 0.94 | 0.34 | 0.83 | 0.15 |
| MG1655-acrAi | 1.11 | 0.15 | 0.98 | 0.12 |
| MG1655-recAi | 1.00 | 0.23 | 0.90 | 0.13 |
| MG1655-dinBi | 1.00 | 0.32 | 1.15 | 0.21 |
| MG1655-marAi | 0.86 | 0.06 | 0.76 | 0.19 |
| MG1655-mutSa | 1.13 | 0.01 | 0.67 | 0.23 |
| MG1655-soxSa | 1.17 | 0.28 | 1.31 | 0.90 |
| MG1655-tolCa | 1.08 | 0.23 | 0.89 | 0.20 |
| MG1655-acrAa | 0.91 | 0.31 | 1.18 | 0.38 |
| MG1655-recAa | 1.11 | 0.22 | 1.09 | 0.26 |
| MG1655-dinBa | 1.16 | 0.31 | 1.10 | 0.30 |
| MG1655-marAa | 1.00 | 0.11 | 1.08 | 0.11 |
| MG1655-tolCi-acrAi | 1.05 | 0.23 | 1.23 | 0.24 |
| MG1655-tolCi-acrAi-soxSi | 1.00 | 0.07 | 0.89 | 0.06 |
| MG1655-recAa-dinBa | 1.15 | 0.19 | 1.02 | 0.40 |
| MG1655-mutSa-dinBi | 1.08 | 0.24 | 0.75 | 0.24 |
| MG1655-tolCi-dinBi | 1.11 | 0.07 | 0.86 | 0.08 |
| Bleach Stress | | | | |
| MG1655-mutSi | 0.71 | 0.53 | 0.91 | 0.24 |
| MG1655-soxSi | 0.91 | 0.50 | 0.78 | 0.18 |
| MG1655-tolCi | 0.61 | 0.64 | 1.31 | 0.93 |
| MG1655-acrAi | 0.88 | 0.29 | 0.31 | 0.18 |
| MG1655-recAi | 0.96 | 0.31 | 0.76 | 0.11 |
| MG1655-dinBi | 0.32 | 0.04 | 0.89 | 0.23 |
| MG1655-marAi | 0.58 | 0.26 | 0.32 | 0.13 |
| MG1655-mutSa | 1.22 | 0.25 | 1.30 | 0.66 |
| MG1655-soxSa | 1.23 | 0.26 | 1.07 | 0.17 |
| MG1655-tolCa | 1.14 | 0.23 | 0.73 | 0.09 |
| MG1655-acrAa | 0.87 | 0.33 | 0.90 | 0.42 |
| MG1655-recAa | 0.82 | 0.49 | 0.64 | 0.13 |
| MG1655-dinBa | 1.29 | 0.42 | 0.53 | 0.05 |
| MG1655-marAa | 0.99 | 0.37 | 0.24 | 0.06 |
| MG1655-tolCi-acrAi | 0.89 | 0.49 | 0.33 | 0.01 |
| MG1655-tolCi-acrAi-soxSi | 0.67 | 0.42 | 0.28 | 0.06 |
| MG1655-recAa-dinBa | 0.75 | 0.16 | 0.31 | 0.06 |
| MG1655-mutSa-dinBi | 0.81 | 0.27 | 0.20 | 0.02 |
| MG1655-tolCi-dinBi | 0.82 | 0.55 | 0.26 | 0.01 |
| Peroxide Stress | | | | |
| MG1655-mutSi | 0.99 | 0.13 | 0.26 | 0.07 |
| MG1655-soxSi | 1.21 | 0.23 | 1.04 | 0.75 |
| MG1655-tolCi | 1.05 | 0.23 | 0.77 | 0.07 |
| MG1655-acrAi | 0.91 | 0.19 | 0.90 | 0.26 |
| MG1655-recAi | 0.76 | 0.11 | 0.73 | 0.22 |
| MG1655-dinBi | 1.01 | 0.20 | 0.61 | 0.08 |
| MG1655-marAi | 0.94 | 0.14 | 0.28 | 0.11 |
| MG1655-mutSa | 1.10 | 0.20 | 1.52 | 0.44 |
| MG1655-soxSa | 1.17 | 0.13 | 1.47 | 0.96 |
| MG1655-tolCa | 1.12 | 0.17 | 1.00 | 0.26 |
| MG1655-acrAa | 1.21 | 0.24 | 0.55 | 0.21 |
| MG1655-recAa | 1.15 | 0.33 | 1.13 | 0.34 |
| MG1655-dinBa | 1.50 | 0.04 | 0.94 | 0.16 |
| MG1655-marAa | 0.92 | 0.02 | 0.41 | 0.13 |
| MG1655-tolCi-acrAi | 1.07 | 0.10 | 0.50 | 0.06 |
| MG1655-tolCi-acrAi-soxSi | 0.94 | 0.07 | 0.39 | 0.10 |
| MG1655-recAa-dinBa | 0.90 | 0.25 | 0.54 | 0.09 |
| MG1655-mutSa-dinBi | 1.21 | 0.33 | 0.73 | 0.02 |
| MG1655-tolCi-dinBi | 1.17 | 0.09 | 0.58 | 0.12 |
| Tetracycline Stress | | | | |
| MG1655-mutSi | 1.52 | 0.24 | 1.62 | 1.13 |
| MG1655-soxSi | 1.24 | 0.35 | 1.53 | 0.27 |
| MG1655-tolCi | 0.69 | 0.28 | 2.31 | 0.82 |
| MG1655-acrAi | 1.12 | 0.27 | 1.83 | 0.27 |
| MG1655-recAi | 0.73 | 0.20 | 2.62 | 1.75 |
| MG1655-dinBi | 0.60 | 0.38 | 2.68 | 0.65 |
| MG1655-marAi | 0.60 | 0.31 | 0.46 | 0.13 |
| MG1655-mutSa | 0.88 | 0.44 | 4.49 | 2.05 |
| MG1655-soxSa | 1.19 | 0.25 | 2.78 | 0.73 |
| MG1655-tolCa | 1.34 | 0.09 | 7.34 | 4.70 |
| MG1655-acrAa | 1.03 | 0.39 | 5.05 | 3.14 |
| MG1655-recAa | 1.38 | 0.15 | 0.25 | 0.10 |
| MG1655-dinBa | 1.04 | 0.38 | 0.72 | 0.08 |
| MG1655-marAa | 0.99 | 0.03 | 0.16 | 0.01 |
| MG1655-tolCi-acrAi | 0.61 | 0.08 | 0.52 | 0.06 |
| MG1655-tolCi-acrAi-soxSi | 0.67 | 0.06 | 1.18 | 0.97 |
| MG1655-recAa-dinBa | 1.04 | 0.24 | 0.49 | 0.10 |
| MG1655-mutSa-dinBi | 1.15 | 0.11 | 0.67 | 0.18 |
| MG1655-tolCi-dinBi | 0.92 | 0.36 | 0.59 | 0.16 |
| Rifampicin Stress | | | | |
| MG1655-mutSi | 0.95 | 0.11 | 0.81 | 0.22 |
| MG1655-soxSi | 0.70 | 0.10 | 1.09 | 0.43 |
| MG1655-tolCi | 0.49 | 0.30 | 1.11 | 0.82 |
| MG1655-acrAi | 0.68 | 0.15 | 1.26 | 0.32 |
| MG1655-recAi | 0.60 | 0.07 | 0.60 | 0.19 |
| MG1655-dinBi | 0.72 | 0.38 | 1.00 | 0.19 |
| MG1655-marAi | 0.60 | 0.26 | 0.35 | 0.15 |
| MG1655-matSa | 1.24 | 0.14 | 0.66 | 0.26 |
| MG1655-soxSa | 1.43 | 0.13 | 0.41 | 0.04 |
| MG1655-tolCa | 1.40 | 0.40 | 0.47 | 0.02 |
| MG1655-acrAa | 1.33 | 0.25 | 1.50 | 0.93 |
| MG1655-recAa | 0.91 | 0.16 | 1.95 | 1.13 |
| MG1655-dinBa | 0.95 | 0.06 | 0.67 | 0.10 |
| MG1655-marAa | 0.93 | 0.12 | 0.56 | 0.19 |
| MG1655-tolCi-acrAi | 0.69 | 0.15 | 0.57 | 0.09 |
| MG1655-tolCi-acrAi-soxSi | 0.73 | 0.15 | 0.85 | 0.21 |
| MG1655-recAa-dinBa | 0.77 | 0.16 | 0.95 | 0.26 |
| MG1655-mutSa-dinBi | 0.81 | 0.02 | 0.83 | 0.14 |
| MG1655-tolCi-dinBi | 0.74 | 0.36 | 1.52 | 0.41 |
| Glucose Limitation Stress | | | | |
| MG1655-mutSi | 1.00 | 0.23 | 1.10 | 0.16 |
| MG1655-soxSi | 1.00 | 0.20 | 1.19 | 0.32 |
| MG1655-tolCi | 0.75 | 0.14 | 0.61 | 0.12 |
| MG1655-acrAi | 0.88 | 0.16 | 1.12 | 0.67 |
| MG1655-recAi | 0.77 | 0.12 | 1.52 | 0.19 |
| MG1655-dinBi | 1.10 | 0.15 | 0.63 | 0.04 |
| MG1655-marAi | 0.95 | 0.16 | 1.83 | 0.86 |
| MG1655-mutSa | 1.03 | 0.34 | 1.07 | 0.16 |
| MG1655-soxSa | 1.02 | 0.42 | 1.15 | 0.06 |
| MG1655-tolCa | 0.93 | 0.35 | 1.22 | 0.35 |
| MG1655-acrAa | 0.66 | 0.21 | 1.07 | 0.61 |
| MG1655-recAa | 0.86 | 0.18 | 2.34 | 1.44 |
| MG1655-dinBa | 0.59 | 0.12 | 0.94 | 0.45 |
| MG1655-marAa | 0.78 | 0.22 | 0.98 | 0.31 |
| MG1655-tolCi-acrAi | 0.64 | 0.03 | 0.75 | 0.05 |
| MG1655-tolCi-acrAi-soxSi | 0.67 | 0.03 | 1.11 | 0.68 |
| MG1655-recAa-dinBa | 2.32 | 2.24 | 0.54 | 0.13 |
| MG1655-mutSa-dinBi | 0.75 | 0.14 | 1.00 | 0.31 |
| MG1655-tolCi-dinBi | 0.97 | 0.06 | 0.96 | 0.40 |

TABLE 8
Day 2 calculations for average normalized growth rate and normalized inverse lag

| | Average Normalized | StDev Normalized | Average Normalized | StDev Normalized |
|---|---|---|---|---|
| | Growth Rate | Growth Rate | Inverse Lag Time | Inverse Lag Time |
| No Stress | | | | |
| MG1655-mutSi | 0.85 | 0.22 | 1.04 | 0.19 |
| MG1655-soxSi | 0.87 | 0.23 | 1.24 | 0.13 |
| MG1655-tolCi | 0.99 | 0.02 | 0.88 | 0.22 |
| MG1655-acrAi | 1.11 | 0.15 | 1.03 | 0.10 |
| MG1655-recAi | 1.12 | 0.04 | 0.92 | 0.17 |
| MG1655-dinBi | 1.20 | 0.07 | 0.90 | 0.15 |
| MG1655-marAi | 1.15 | 0.19 | 0.90 | 0.19 |
| MG1655-mutSa | 1.08 | 0.05 | 0.98 | 0.23 |
| MG1655-soxSa | 1.20 | 0.15 | 0.98 | 0.24 |
| MG1655-tolCa | 1.04 | 0.20 | 0.80 | 0.08 |
| MG1655-acrAa | 0.93 | 0.14 | 1.12 | 0.30 |
| MG1655-recAa | 1.04 | 0.18 | 0.98 | 0.16 |
| MG1655-dinBa | 1.21 | 0.12 | 0.92 | 0.12 |
| MG1655-marAa | 0.99 | 0.10 | 0.81 | 0.13 |
| MG1655-tolCi-acrAi | 1.14 | 0.18 | 1.07 | 0.28 |
| MG1655-tolCi-acrAi-soxSi | 1.02 | 0.14 | 1.78 | 0.39 |
| MG1655-recAa-dinBa | 1.16 | 0.07 | 1.20 | 0.10 |
| MG1655-mutSa-dinBi | 1.16 | 0.11 | 1.31 | 0.15 |
| MG1655-tolCi-dinBi | 1.18 | 0.10 | 1.90 | 0.37 |
| Bleach Stress | | | | |
| MG1655-mutSi | 0.64 | 0.01 | 3.87 | 2.52 |
| MG1655-soxSi | 0.94 | 0.24 | 1.76 | 0.26 |
| MG1655-tolCi | 0.63 | 0.13 | 1.32 | 1.00 |
| MG1655-acrAi | 0.93 | 0.20 | 0.75 | 0.27 |
| MG1655-recAi | 0.92 | 0.16 | 0.92 | 0.20 |
| MG1655-dinBi | 0.58 | 0.28 | 1.38 | 0.42 |
| MG1655-marAi | 1.04 | 0.28 | 0.70 | 0.20 |
| MG1655-mutSa | 1.01 | 0.18 | 1.69 | 1.39 |
| MG1655-soxSa | 0.87 | 0.13 | 5.73 | 5.52 |
| MG1655-tolCa | 0.89 | 0.18 | 2.04 | 1.90 |
| MG1655-acrAa | 0.84 | 0.22 | 0.68 | 0.22 |
| MG1655-recAa | 1.08 | 0.23 | 1.04 | 0.13 |
| MG1655-dinBa | 0.64 | 0.02 | 1.46 | 0.27 |
| MG1655-marAa | 1.06 | 0.33 | 0.71 | 0.11 |
| MG1655-tolCi-acrAi | 1.04 | 0.33 | 0.97 | 0.09 |
| MG1655-tolCi-acrAi-soxSi | 1.08 | 0.39 | 0.67 | 0.19 |
| MG1655-recAa-dinBa | 1.17 | 0.28 | 1.05 | 0.21 |
| MG1655-mutSa-dinBi | 0.90 | 0.35 | 2.91 | 1.50 |
| MG1655-tolCi-dinBi | 1.12 | 0.32 | 5.89 | 3.42 |
| Peroxide Stress | | | | |
| MG1655-mutSi | 0.80 | 0.12 | 0.81 | 0.26 |
| MG1655-soxSi | 0.73 | 0.11 | 4.08 | 2.36 |
| MG1655-tolCi | 1.03 | 0.26 | 1.82 | 1.19 |
| MG1655-acrAi | 0.72 | 0.16 | 0.67 | 0.22 |
| MG1655-recAi | 0.63 | 0.15 | 1.42 | 1.32 |
| MG1655-dinBi | 0.96 | 0.22 | 2.20 | 1.68 |
| MG1655-marAi | 0.79 | 0.20 | 0.72 | 0.12 |
| MG1655-mutSa | 0.14 | 0.07 | 1.51 | 0.73 |
| MG1655-soxSa | 1.05 | 0.13 | 0.51 | 0.18 |
| MG1655-tolCa | 1.55 | 0.28 | 0.66 | 0.37 |
| MG1655-acrAa | 0.82 | 0.10 | 1.31 | 1.00 |
| MG1655-recAa | 0.91 | 0.23 | 0.88 | 0.44 |
| MG1655-dinBa | 1.08 | 0.18 | 0.97 | 0.15 |
| MG1655-marAa | 0.84 | 0.10 | 0.47 | 0.11 |
| MG1655-tolCi-acrAi | 0.64 | 0.18 | 2.13 | 1.02 |
| MG1655-tolCi-acrAi-soxSi | 0.68 | 0.01 | 0.94 | 0.38 |
| MG1655-recAa-dinBa | 0.95 | 0.19 | 0.94 | 0.28 |
| MG1655-mutSa-dinBi | 1.16 | 0.27 | 0.95 | 0.06 |
| MG1655-tolCi-dinBi | 1.06 | 0.17 | 1.91 | 0.92 |
| Tetracycline Stress | | | | |
| MG1655-mutSi | 1.90 | 0.86 | 0.40 | 0.05 |
| MG1655-soxSi | 1.10 | 0.78 | 1.25 | 0.51 |
| MG1655-tolCi | 0.69 | 0.36 | 0.92 | 0.17 |
| MG1655-acrAi | 1.05 | 0.37 | 1.18 | 0.33 |
| MG1655-recAi | 1.85 | 1.34 | 1.24 | 0.16 |
| MG1655-dinBi | 1.24 | 0.90 | 1.34 | 0.26 |
| MG1655-marAi | 0.88 | 0.08 | 0.31 | 0.05 |
| MG1655-mutSa | 0.83 | 0.49 | 7.83 | 4.91 |
| MG1655-soxSa | 0.73 | 0.24 | 1.62 | 0.83 |
| MG1655-tolCa | 2.11 | 0.93 | 1.34 | 0.56 |
| MG1655-acrAa | 1.07 | 0.39 | 0.96 | 0.49 |
| MG1655-recAa | 2.06 | 0.97 | 0.51 | 0.01 |
| MG1655-dinBa | 3.27 | 0.41 | 4.70 | 1.76 |
| MG1655-marAa | 1.16 | 0.19 | 0.27 | 0.02 |
| MG1655-tolCi-acrAi | 0.98 | 0.12 | 0.54 | 0.08 |
| MG1655-tolCi-acrAi-soxSi | 1.13 | 0.02 | 0.41 | 0.11 |
| MG1655-recAa-dinBa | 0.81 | 0.25 | 0.65 | 0.12 |
| MG1655-mutSa-dinBi | 0.75 | 0.35 | 0.54 | 0.03 |
| MG1655-tolCi-dinBi | 1.05 | 0.20 | 0.42 | 0.07 |
| Rifampicin Stress | | | | |
| MG1655-mutSi | 1.09 | 0.19 | 0.61 | 0.11 |
| MG1655-soxSi | 1.25 | 0.21 | 1.23 | 0.86 |
| MG1655-tolCi | 0.60 | 0.21 | 1.79 | 0.24 |
| MG1655-acrAi | 0.48 | 0.14 | 0.46 | 0.13 |
| MG1655-recAi | 0.48 | 0.17 | 1.04 | 0.45 |
| MG1655-dinBi | 0.70 | 0.37 | 0.98 | 0.82 |
| MG1655-marAi | 0.91 | 0.05 | 0.65 | 0.35 |
| MG1655-mutSa | 1.89 | 0.31 | 1.72 | 1.03 |
| MG1655-soxSa | 1.90 | 0.14 | 0.73 | 0.19 |
| MG1655-tolCa | 1.27 | 0.33 | 0.83 | 0.31 |
| MG1655-acrAa | 1.48 | 0.32 | 0.87 | 0.42 |
| MG1655-recAa | 1.35 | 0.26 | 0.56 | 0.16 |
| MG1655-dinBa | 1.52 | 0.12 | 0.43 | 0.13 |
| MG1655-marAa | 1.14 | 0.30 | 0.65 | 0.13 |
| MG1655-tolCi-acrAi | 1.06 | 0.33 | 0.92 | 0.20 |
| MG1655-tolCi-acrAi-soxSi | 1.46 | 0.22 | 2.78 | 0.95 |
| MG1655-recAa-dinBa | 0.74 | 0.22 | 0.67 | 0.27 |
| MG1655-mutSa-dinBi | 0.88 | 0.06 | 0.58 | 0.25 |
| MG1655-tolCi-dinBi | 0.74 | 0.18 | 2.63 | 1.66 |
| Glucose Limitation Stress | | | | |
| MG1655-mutSi | 1.34 | 0.13 | 3.21 | 1.91 |
| MG1655-soxSi | 0.85 | 0.12 | 1.69 | 0.70 |
| MG1655-tolCi | 1.13 | 0.23 | 0.69 | 0.15 |
| MG1655-acrAi | 0.75 | 0.07 | 0.42 | 0.05 |
| MG1655-recAi | 0.68 | 0.02 | 0.76 | 0.28 |
| MG1655-dinBi | 0.56 | 0.02 | 2.48 | 0.31 |
| MG1655-marAi | 1.16 | 0.46 | 1.67 | 1.18 |
| MG1655-mutSa | 1.29 | 0.12 | 0.94 | 0.18 |
| MG1655-soxSa | 0.78 | 0.25 | 1.22 | 0.08 |
| MG1655-tolCa | 1.44 | 0.08 | 0.73 | 0.32 |
| MG1655-acrAa | 0.50 | 0.20 | 0.35 | 0.14 |
| MG1655-recAa | 0.87 | 0.07 | 0.96 | 0.33 |
| MG1655-dinBa | 1.01 | 0.10 | 0.74 | 0.32 |
| MG1655-marAa | 0.97 | 0.35 | 0.24 | 0.08 |
| MG1655-tolCi-acrAi | 1.07 | 0.20 | 0.50 | 0.03 |
| MG1655-tolCi-acrAi-soxSi | 1.16 | 0.17 | 0.52 | 0.21 |
| MG1655-recAa-dinBa | 0.87 | 0.16 | 0.41 | 0.06 |
| MG1655-mutSa-dinBi | 0.93 | 0.45 | 0.34 | 0.13 |
| MG1655-tolCi-dinBi | 1.04 | 0.22 | 0.88 | 0.29 |

TABLE 9
Day 3 calculations for average normalized growth rate and normalized inverse lag

| | Average Normalized | StDev Normalized | Average Normalized | StDev Normalized |
|---|---|---|---|---|
| | Growth Rate | Growth Rate | Inverse Lag Time | Inverse Lag Time |
| No Stress | | | | |
| MG1655-mutSi | 0.93 | 0.26 | 0.84 | 0.12 |
| MG1655-soxSi | 0.96 | 0.31 | 1.00 | 0.15 |
| MG1655-tolCi | 0.91 | 0.17 | 0.86 | 0.06 |
| MG1655-acrAi | 1.01 | 0.20 | 1.05 | 0.02 |
| MG1655-recAi | 0.79 | 0.17 | 0.87 | 0.05 |
| MG1655-dinBi | 0.84 | 0.13 | 0.90 | 0.19 |
| MG1655-marAi | 0.74 | 0.22 | 1.05 | 0.15 |
| MG1655-mutSa | 1.00 | 0.16 | 1.04 | 0.04 |
| MG1655-soxSa | 1.10 | 0.15 | 1.11 | 0.45 |
| MG1655-tolCa | 1.04 | 0.18 | 0.97 | 0.09 |
| MG1655-acrAa | 1.02 | 0.11 | 1.06 | 0.29 |
| MG1655-recAa | 0.91 | 0.12 | 1.00 | 0.12 |
| MG1655-dinBa | 1.08 | 0.15 | 1.04 | 0.17 |
| MG1655-marAa | 1.05 | 0.07 | 1.11 | 0.18 |
| MG1655-tolCi-acrAi | 0.95 | 0.27 | 1.11 | 0.21 |
| MG1655-tolCi-acrAi-soxSi | 0.91 | 0.19 | 1.12 | 0.36 |
| MG1655-recAa-dinBa | 1.05 | 0.17 | 0.92 | 0.18 |
| MG1655-mutSa-dinBi | 1.00 | 0.12 | 1.33 | 0.17 |
| MG1655-tolCi-dinBi | 0.84 | 0.26 | 0.88 | 0.26 |
| Bleach Stress | | | | |
| MG1655-mutSi | 1.22 | 0.37 | 1.51 | 0.30 |
| MG1655-soxSi | 0.98 | 0.17 | 0.87 | 0.05 |
| MG1655-tolCi | 1.04 | 0.09 | 0.73 | 0.19 |
| MG1655-acrAi | 1.02 | 0.30 | 0.97 | 0.13 |
| MG1655-recAi | 0.89 | 0.20 | 1.03 | 0.37 |
| MG1655-dinBi | 0.62 | 0.12 | 0.99 | 0.36 |
| MG1655-marAi | 0.83 | 0.12 | 0.69 | 0.20 |
| MG1655-mutSa | 1.01 | 0.29 | 0.75 | 0.20 |
| MG1655-soxSa | 0.65 | 0.09 | 1.98 | 0.51 |
| MG1655-tolCa | 0.94 | 0.46 | 0.72 | 0.22 |
| MG1655-acrAa | 0.81 | 0.15 | 0.82 | 0.14 |
| MG1655-recAa | 0.97 | 0.22 | 0.86 | 0.35 |
| MG1655-dinBa | 0.92 | 0.07 | 1.07 | 0.44 |
| MG1655-marAa | 0.97 | 0.15 | 0.46 | 0.16 |
| MG1655-tolCi-acrAi | 0.89 | 0.22 | 0.90 | 0.27 |
| MG1655-tolCi-acrAi-soxSi | 0.79 | 0.10 | 0.69 | 0.15 |
| MG1655-recAa-dinBa | 1.07 | 0.19 | 0.67 | 0.29 |
| MG1655-mutSa-dinBi | 0.96 | 0.13 | 0.65 | 0.32 |
| MG1655-tolCi-dinBi | 0.70 | 0.26 | 0.73 | 0.45 |
| Peroxide Stress | | | | |
| MG1655-mutSi | 0.90 | 0.08 | 0.76 | 0.08 |
| MG1655-soxSi | 0.94 | 0.03 | 0.95 | 0.24 |
| MG1655-tolCi | 0.98 | 0.03 | 0.42 | 0.12 |
| MG1655-acrAi | 0.90 | 0.14 | 0.66 | 0.11 |
| MG1655-recAi | 0.76 | 0.16 | 0.58 | 0.06 |
| MG1655-dinBi | 0.92 | 0.04 | 0.56 | 0.10 |
| MG1655-marAi | 0.88 | 0.04 | 0.96 | 0.28 |
| MG1655-mutSa | 0.85 | 0.02 | 1.14 | 0.10 |
| MG1655-soxSa | 1.05 | 0.16 | 0.89 | 0.08 |
| MG1655-tolCa | 0.94 | 0.13 | 1.48 | 0.18 |
| MG1655-acrAa | 1.05 | 0.13 | 0.92 | 0.48 |
| MG1655-recAa | 1.09 | 0.16 | 1.15 | 0.26 |
| MG1655-dinBa | 0.82 | 0.05 | 1.14 | 0.31 |
| MG1655-marAa | 0.69 | 0.10 | 0.39 | 0.17 |
| MG1655-tolCi-acrAi | 0.67 | 0.12 | 0.90 | 0.34 |
| MG1655-tolCi-acrAi-soxSi | 0.78 | 0.19 | 0.89 | 0.09 |
| MG1655-recAa-dinBa | 0.81 | 0.14 | 0.65 | 0.25 |
| MG1655-mutSa-dinBi | 0.85 | 0.11 | 0.83 | 0.32 |
| MG1655-tolCi-dinBi | 0.75 | 0.03 | 0.60 | 0.16 |
| Tetracycline Stress | | | | |
| MG1655-mutSi | 1.61 | 0.17 | 0.76 | 0.32 |
| MG1655-soxSi | 1.07 | 0.33 | 0.39 | 0.14 |
| MG1655-tolCi | 0.59 | 0.20 | 0.72 | 0.22 |
| MG1655-acrAi | 1.18 | 0.09 | 0.79 | 0.17 |
| MG1655-recAi | 0.36 | 0.22 | 1.47 | 0.08 |
| MG1655-dinBi | 0.34 | 0.06 | 0.80 | 0.31 |
| MG1655-marAi | 0.35 | 0.08 | 0.97 | 0.02 |
| MG1655-mutSa | 0.42 | 0.46 | 1.52 | 0.51 |
| MG1655-soxSa | 1.97 | 0.43 | 1.59 | 0.35 |
| MG1655-tolCa | 2.14 | 0.92 | 0.99 | 0.01 |
| MG1655-acrAa | 1.08 | 0.80 | 1.40 | 0.25 |
| MG1655-recAa | 0.79 | 0.38 | 0.49 | 0.34 |
| MG1655-dinBa | 2.11 | 0.64 | 1.14 | 0.13 |
| MG1655-marAa | 1.37 | 0.22 | 0.69 | 0.55 |
| MG1655-tolCi-acrAi | 0.49 | 0.43 | 0.26 | 0.22 |
| MG1655-tolCi-acrAi-soxSi | 0.54 | 0.06 | 0.38 | 0.15 |
| MG1655-recAa-dinBa | 0.75 | 0.44 | 0.90 | 0.10 |
| MG1655-mutSa-dinBi | 0.98 | 0.48 | 0.64 | 0.32 |
| MG1655-tolCi-dinBi | 0.56 | 0.42 | 0.49 | 0.05 |
| Rifampicin Stress | | | | |
| MG1655-mutSi | 1.11 | 0.28 | 0.36 | 0.01 |
| MG1655-soxSi | 0.90 | 0.02 | 0.60 | 0.17 |
| MG1655-tolCi | 0.64 | 0.15 | 0.35 | 0.17 |
| MG1655-acrAi | 0.71 | 0.12 | 0.73 | 0.08 |
| MG1655-recAi | 1.10 | 0.16 | 0.49 | 0.12 |
| MG1655-dinBi | 0.97 | 0.12 | 1.28 | 0.19 |
| MG1655-marAi | 0.78 | 0.15 | 0.68 | 0.04 |
| MG1655-mutSa | 1.41 | 0.15 | 3.01 | 0.55 |
| MG1655-soxSa | 1.37 | 0.08 | 0.75 | 0.30 |
| MG1655-tolCa | 1.23 | 0.19 | 0.68 | 0.27 |
| MG1655-acrAa | 1.25 | 0.18 | 1.62 | 0.25 |
| MG1655-recAa | 0.87 | 0.02 | 0.86 | 0.36 |
| MG1655-dinBa | 1.05 | 0.11 | 0.31 | 0.20 |
| MG1655-marAa | 1.30 | 0.12 | 0.79 | 0.11 |
| MG1655-tolCi-acrAi | 0.88 | 0.11 | 0.62 | 0.31 |
| MG1655-tolCi-acrAi-soxSi | 1.00 | 0.17 | 0.81 | 0.20 |
| MG1655-recAa-dinBa | 0.83 | 0.10 | 0.49 | 0.25 |
| MG1655-mutSa-dinBi | 0.78 | 0.23 | 0.80 | 0.54 |
| MG1655-tolCi-dinBi | 0.90 | 0.31 | 0.47 | 0.25 |
| Glucose Limitation Stress | | | | |
| MG1655-mutSi | 1.28 | 0.21 | 1.00 | 0.09 |
| MG1655-soxSi | 1.29 | 0.21 | 0.86 | 0.11 |
| MG1655-tolCi | 1.11 | 0.10 | 1.11 | 0.07 |
| MG1655-acrAi | 1.24 | 0.18 | 0.73 | 0.27 |
| MG1655-recAi | 1.21 | 0.35 | 0.65 | 0.10 |
| MG1655-dinBi | 1.15 | 0.13 | 0.94 | 0.05 |
| MG1655-marAi | 1.12 | 0.19 | 0.65 | 0.26 |
| MG1655-mutSa | 0.87 | 0.12 | 0.89 | 0.33 |
| MG1655-soxSa | 1.21 | 0.01 | 0.64 | 0.14 |
| MG1655-tolCa | 0.83 | 0.04 | 0.87 | 0.18 |
| MG1655-acrAa | 0.93 | 0.20 | 0.93 | 0.14 |
| MG1655-recAa | 0.71 | 0.08 | 0.93 | 0.14 |
| MG1655-dinBa | 0.97 | 0.04 | 1.07 | 0.14 |
| MG1655-marAa | 0.96 | 0.05 | 1.20 | 0.10 |
| MG1655-tolCi-acrAi | 0.77 | 0.08 | 1.35 | 0.31 |
| MG1655-tolCi-acrAi-soxSi | 0.88 | 0.08 | 0.91 | 0.30 |
| MG1655-recAa-dinBa | 0.88 | 0.40 | 0.80 | 0.22 |
| MG1655-mutSa-dinBi | 0.85 | 0.24 | 0.83 | 0.12 |
| MG1655-tolCi-dinBi | 0.84 | 0.15 | 0.88 | 0.28 |

TABLE 10
3-day average of normalized growth rate and normalized inverse lag time.

| | Average Normalized | StDev Normalized | Average Normalized | StDev Normalized |
|---|---|---|---|---|
| | Growth Rate | Growth Rate | Inverse Lag Time | Inverse Lag Time |
| No Stress | | | | |
| MG1655-mutSi | 1.05 | 0.01 | 0.90 | 0.03 |
| MG1655-soxSi | 1.09 | 0.01 | 1.03 | 0.01 |
| MG1655-tolCi | 1.07 | 0.07 | 0.94 | 0.03 |
| MG1655-acrAi | 1.06 | 0.06 | 0.98 | 0.01 |
| MG1655-recAi | 1.11 | 0.10 | 0.99 | 0.14 |
| MG1655-dinBi | 1.04 | 0.08 | 1.05 | 0.17 |
| MG1655-marAi | 1.00 | 0.15 | 0.66 | 0.04 |
| MG1655-mutSa | 0.98 | 0.02 | 0.99 | 0.15 |
| MG1655-soxSa | 0.99 | 0.04 | 0.98 | 0.17 |
| MG1655-tolCa | 0.95 | 0.04 | 1.00 | 0.12 |
| MG1655-acrAa | 0.88 | 0.06 | 0.94 | 0.14 |
| MG1655-recAa | 0.91 | 0.07 | 1.11 | 0.07 |
| MG1655-dinBa | 1.02 | 0.09 | 0.86 | 0.14 |
| MG1655-marAa | 0.89 | 0.05 | 0.95 | 0.10 |
| MG1655-tolCi-acrAi | 1.16 | 0.12 | 1.20 | 0.19 |
| MG1655-tolCi-acrAi-soxSi | 1.10 | 0.14 | 0.97 | 0.10 |
| MG1655-recAa-dinBa | 1.01 | 0.08 | 0.90 | 0.08 |
| MG1655-mutSa-dinBi | 0.92 | 0.03 | 0.89 | 0.09 |
| MG1655-tolCi-dinBi | 1.03 | 0.12 | 0.95 | 0.17 |
| Bleach Stress | | | | |
| MG1655-mutSi | 1.04 | 0.11 | 1.40 | 0.02 |
| MG1655-soxSi | 1.02 | 0.06 | 1.52 | 0.19 |
| MG1655-tolCi | 0.90 | 0.12 | 0.83 | 0.10 |
| MG1655-acrAi | 1.10 | 0.05 | 1.06 | 0.17 |
| MG1655-recAi | 0.97 | 0.06 | 0.87 | 0.09 |
| MG1655-dinBi | 0.76 | 0.14 | 1.44 | 0.05 |
| MG1655-marAi | 0.85 | 0.07 | 0.80 | 0.06 |
| MG1655-mutSa | 1.18 | 0.10 | 1.13 | 0.11 |
| MG1655-soxSa | 1.03 | 0.06 | 1.54 | 0.22 |
| MG1655-tolCa | 1.07 | 0.11 | 0.60 | 0.10 |
| MG1655-acrAa | 0.90 | 0.19 | 0.70 | 0.20 |
| MG1655-recAa | 1.01 | 0.07 | 0.73 | 0.13 |
| MG1655-dinBa | 1.06 | 0.03 | 0.85 | 0.09 |
| MG1655-marAa | 1.01 | 0.04 | 0.46 | 0.01 |
| MG1655-tolCi-acrAi | 0.86 | 0.08 | 0.83 | 0.06 |
| MG1655-tolCi-acrAi-soxSi | 0.79 | 0.08 | 0.95 | 0.27 |
| MG1655-recAa-dinBa | 1.05 | 0.11 | 0.63 | 0.08 |
| MG1655-mutSa-dinBi | 0.89 | 0.13 | 0.80 | 0.06 |
| MG1655-tolCi-dinBi | 0.75 | 0.21 | 1.38 | 0.26 |
| Peroxide Stress | | | | |
| MG1655-mutSi | 0.90 | 0.07 | 0.47 | 0.06 |
| MG1655-soxSi | 0.96 | 0.09 | 1.56 | 0.23 |
| MG1655-tolCi | 1.02 | 0.14 | 0.71 | 0.06 |
| MG1655-acrAi | 0.83 | 0.08 | 0.83 | 0.07 |
| MG1655-recAi | 0.75 | 0.07 | 1.12 | 0.26 |
| MG1655-dinBi | 0.97 | 0.10 | 0.62 | 0.06 |
| MG1655-marAi | 0.92 | 0.04 | 0.45 | 0.07 |
| MG1655-mutSa | 1.04 | 0.12 | 1.11 | 0.30 |
| MG1655-soxSa | 0.91 | 0.14 | 0.85 | 0.25 |
| MG1655-tolCa | 0.94 | 0.10 | 0.94 | 0.34 |
| MG1655-acrAa | 0.97 | 0.08 | 0.99 | 0.15 |
| MG1655-recAa | 0.99 | 0.12 | 0.84 | 0.04 |
| MG1655-dinBa | 1.11 | 0.03 | 0.98 | 0.11 |
| MG1655-mt7r4a | 0.78 | 0.04 | 0.48 | 0.10 |
| MG1655-tolCi-acrAi | 0.89 | 0.02 | 0.91 | 0.19 |
| MG1655-tolCi-acrAi-soxSi | 0.89 | 0.07 | 0.74 | 0.21 |
| MG1655-recAa-dinBa | 0.84 | 0.03 | 0.72 | 0.12 |
| MG1655-mutSa-dinBi | 1.01 | 0.12 | 0.85 | 0.04 |
| MG1655-tolCi-dinnBi | 0.99 | 0.06 | 0.67 | 0.17 |
| Tetracycline Stress | | | | |
| MG1655-mutSi | 1.17 | 0.05 | 0.72 | 0.12 |
| MG1655-soxSi | 1.14 | 0.06 | 1.33 | 0.18 |
| MG1655-tolCi | 1.04 | 0.15 | 1.16 | 0.25 |
| MG1655-acrAi | 1.05 | 0.07 | 1.37 | 0.25 |
| MG1655-recAi | 1.02 | 0.15 | 1.44 | 0.22 |
| MG1655-dinBi | 1.38 | 0.16 | 1.45 | 0.47 |
| MG1655-marAi | 0.93 | 0.07 | 0.43 | 0.12 |
| MG1655-mutSa | 1.07 | 0.03 | 3.68 | 0.73 |
| MG1655-soxSa | 0.94 | 0.11 | 1.72 | 0.18 |
| MG1655-tolCa | 1.13 | 0.11 | 1.70 | 0.34 |
| MG1655-acrAa | 1.21 | 0.12 | 1.70 | 0.28 |
| MG1655-recAa | 1.72 | 0.26 | 0.46 | 0.12 |
| MG1655-dinBa | 1.67 | 0.20 | 1.19 | 0.11 |
| MG1655-marAa | 2.01 | 0.77 | 0.26 | 0.02 |
| MG1655-tolCi-acrAi | 0.77 | 0.17 | 0.37 | 0.02 |
| MG1655-tolCi-acrAi-soxSi | 1.07 | 0.13 | 0.58 | 0.06 |
| MG1655-recAa-dinBa | 0.79 | 0.27 | 0.63 | 0.22 |
| MG1655-mutSa-dinBi | 1.32 | 0.53 | 0.69 | 0.09 |
| MG1655-tolCi-dinBi | 1.05 | 0.04 | 0.54 | 0.06 |
| Rifampicin Stress | | | | |
| MG1655-mutSi | 1.04 | 0.21 | 0.94 | 0.04 |
| MG1655-soxSi | 1.18 | 0.11 | 1.59 | 0.55 |
| MG1655-tolCi | 1.09 | 0.15 | 1.40 | 0.10 |
| MG1655-acrAi | 0.62 | 0.07 | 0.72 | 0.05 |
| MG1655-recAi | 0.98 | 0.09 | 0.74 | 0.07 |
| MG1655-dinBi | 0.88 | 0.19 | 1.00 | 0.02 |
| MG1655-marAi | 0.89 | 0.06 | 0.55 | 0.16 |
| MG1655-mutSa | 1.38 | 0.12 | 1.40 | 0.16 |
| MG1655-soxSa | 1.35 | 0.10 | 1.03 | 0.73 |
| MG1655-tolCa | 1.03 | 0.02 | 0.65 | 0.10 |
| MG1655-acrAa | 0.86 | 0.07 | 1.58 | 0.32 |
| MG1655-recAa | 0.86 | 0.13 | 1.49 | 0.34 |
| MG1655-dinBa | 1.09 | 0.12 | 0.61 | 0.05 |
| MG1655-marAa | 0.80 | 0.09 | 0.70 | 0.26 |
| MG1655-tolCi-acrAi | 0.90 | 0.05 | 0.78 | 0.06 |
| MG1655-tolCi-acrAi-soxSi | 0.89 | 0.12 | 1.19 | 0.10 |
| MG1655-recAa-dinBa | 0.76 | 0.15 | 0.66 | 0.09 |
| MG1655-mutSa-dinBi | 0.77 | 0.08 | 0.77 | 0.05 |
| MG1655-tolCi-dinBi | 0.89 | 0.10 | 1.92 | 0.22 |
| Glucose Limitation Stress | | | | |
| MG1655-mutSi | 1.18 | 0.10 | 1.87 | 0.47 |
| MG1655-soxSi | 1.09 | 0.02 | 1.52 | 0.19 |
| MG1655-tolCi | 1.25 | 0.06 | 0.88 | 0.33 |
| MG1655-acrAi | 0.89 | 0.06 | 0.69 | 0.06 |
| MG1655-recAi | 0.92 | 0.04 | 0.77 | 0.09 |
| MG1655-dinBi | 1.00 | 0.03 | 0.84 | 0.02 |
| MG1655-marAi | 1.09 | 0.07 | 1.14 | 0.23 |
| MG1655-mutSa | 1.11 | 0.06 | 1.03 | 0.17 |
| MG1655-soxSa | 1.05 | 0.14 | 1.19 | 0.06 |
| MG1655-tolCa | 1.05 | 0.09 | 0.81 | 0.04 |
| MG1655-acrAa | 0.65 | 0.06 | 0.59 | 0.19 |
| MG1655-recAa | 0.93 | 0.10 | 1.21 | 0.17 |
| MG1655-dinBa | 1.01 | 0.12 | 0.86 | 0.10 |
| MG1655-marAa | 0.92 | 0.09 | 0.59 | 0.17 |
| MG1655-tolCi-acrAi | 0.85 | 0.07 | 0.86 | 0.04 |
| MG1655-tolCi-acrAi-soxSi | 1.05 | 0.07 | 0.75 | 0.23 |
| MG1655-recAa-dinBa | 0.89 | 0.05 | 0.54 | 0.14 |
| MG1655-mutSa-dinBi | 0.77 | 0.12 | 0.68 | 0.26 |
| MG1655-tolCi-dinBi | 1.07 | 0.07 | 0.88 | 0.14 |

### Example 8—Engineering Epigenetic Epistasis to Deter Bacterial Adaptation

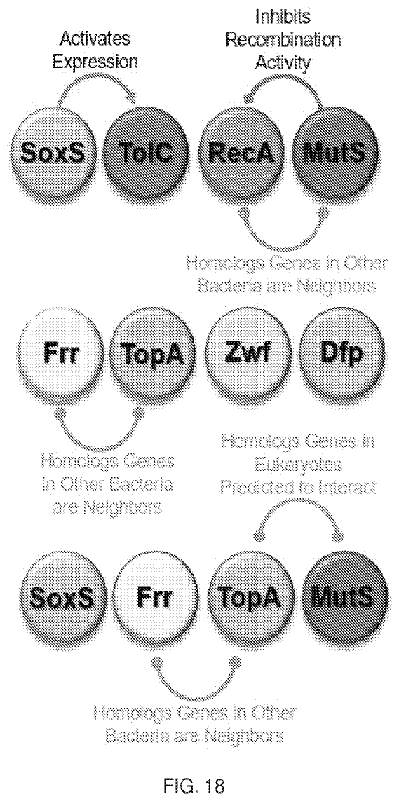

To investigate epistatic effects arising from changes in gene expression of genes that are not known to have any inherent interactions, a set of deactivated CRISPR-Cas9 devices were developed to selectively inhibit or activate expression of two sets of four genes each in Escherichia coli MG1655 (FIG. 17). The first set included activation of four genes commonly involved in bacterial stress response and identified as potentially important players in adaptation: mutS (DNA mismatch repair), soxS (SOX pathway regulator), tolC (multidrug efflux pump) and recA (SOS response activator). The second set included inhibition of genes “central” to different cellular pathways. For this, four highly conserved genes across all bacterial genomes were chosen: dfp (essential for Coenzyme A synthesis), topA (essential for relaxing DNA supercoiling), zwf (a key glycolysis enzyme) and frr (ribosome recycling factor). These genes are monocistronic (except for to/C and recA) and have no known interactions between each other (except for mutS/recA and soxS/tolC) (see FIG. 18). This approach captured the effect of stochastic expression increases.

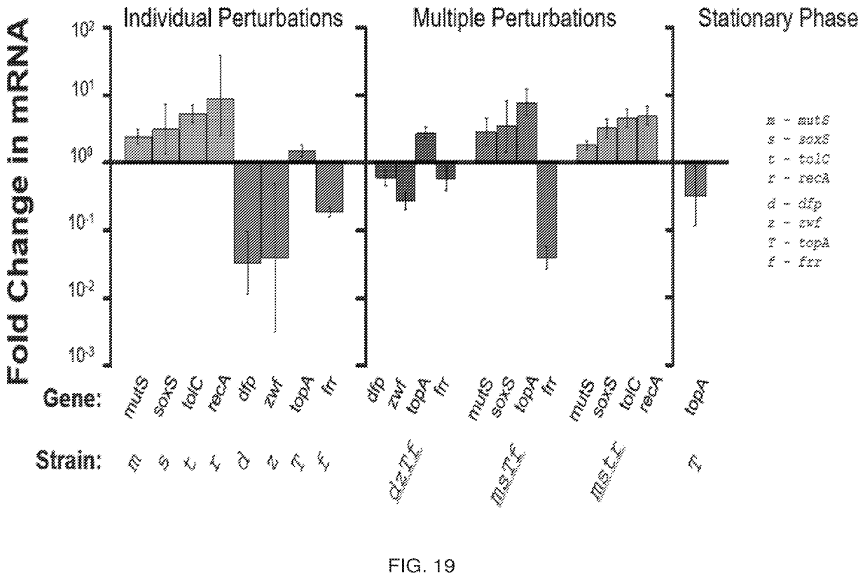

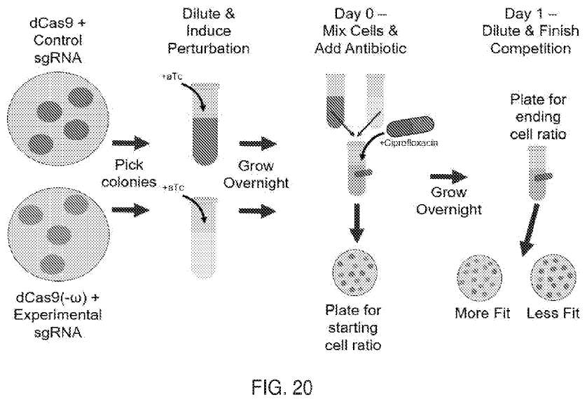

All possible combinations of gene perturbation in each set were constructed, and their influence on gene expression was verified using RT-qPCR (see FIG. 19). topA perturbation was observed to be phase-dependent, inhibiting only during stationary phase but activating during exponential phase inhibition and activation appears to only exhibit a decrease in gene expression during the stationary phase. This is likely due to interference with native phase-dependent Fis regulation of topA. Two control strains were constructed, where “nonsense” gene perturbations directed at one or four copies of rfp, as well as another control strain including constitutively expressed mCherry and rfp perturbation. This final control was competed independently against all 31 experimental strains and the other two controls during antibiotic exposure (see FIG. 20, which depicts the experimental procedure for determining fitness value). Ciprofloxacin was chosen as it is a common antibiotic treatment which also selects for resistant populations at very low concentrations and could demonstrate epigenetic epistasis in a clinically relevant setting.

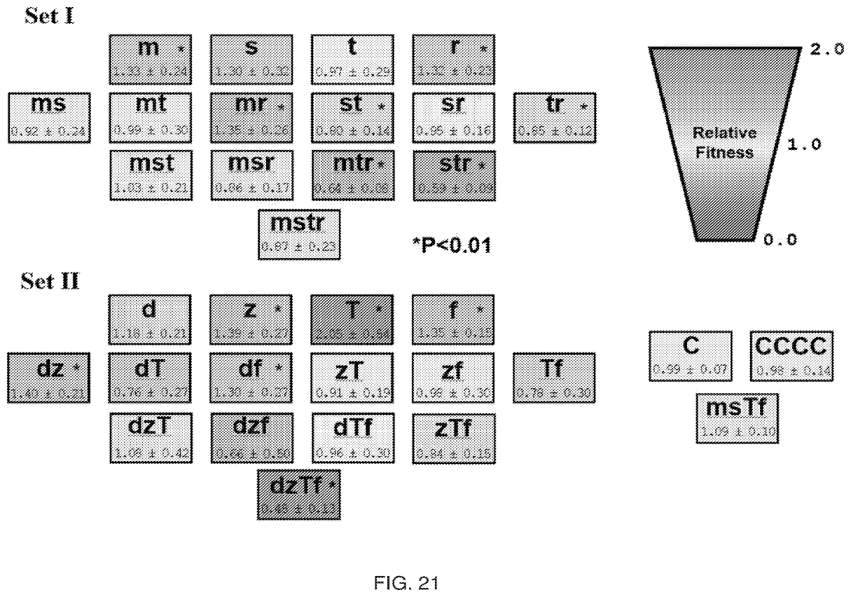

Competition revealed the overall fitness impacts of each possible combination of gene perturbation during sub-MIC ciprofloxacin exposure (FIG. 21). FIG. 21 depicts the fitness impacts of gene expression of gene expression perturbations. Strain names are abbreviated based on present gene perturbations as follows: m—mutS, s—soxS, t—to/C, r—recA, d—dfp, z—zwf, T—topA, f—frr, C—rfp. Gene expression was enhanced in set I and inhibited in set II (except topA whose perturbation was phase dependent, see FIG. 19). Relative finesses are listed below strain names, followed by s.d. (n=8). Asterisks indicate significant fitness differences in relation to strain “C” (P<0.01, two-tailed type II t-test).

Five individual perturbations resulted in an increase in fitness—topA perturbation in particular led to a ˜2-fold increase in fitness likely due to excess TopA acting to recover DNA unfolding inhibited by ciprofloxacin. There was a trend towards lower fitness with more perturbations—simultaneous perturbation of two genes produced a roughly even split of three beneficial and two detrimental significant fitness changes, while all three statistically significant fitness changes in three or four perturbation strains were detrimental. It was useful to look at the average fitness across the population of zero (controls) to four gene perturbations. In doing so, a notable shift in the data was observed as more perturbations were introduced. The average fitness of all single perturbations (1.36±0.40) was statistically greater than the control (0.99±0.10, P=0.0005), but two perturbations aggravated the average fitness back to control-level values (0.99±0.31, P=0.88). Further perturbations intensified this reduction in fitness (0.83±0.32, P=0.06 for three perturbations and 0.81±0.30, P=0.03 for four perturbations). This same trend was observed when dividing strains into the aforementioned sets, with set I (1.22±0.30, P=0.003; 0.98±0.27, P=0.88; 0.78±0.23, P=0.002; 0.87±0.23, P=0.10, one to four perturbations respectively) exhibiting a weaker degree of fitness deviations than set II (1.49±0.44, P=8*10⁻⁵; 1.02±0.34, P=0.71; 0.88±0.38, P=0.29; 0.48±0.13, P=1*10⁻¹¹, one to four perturbations respectively).

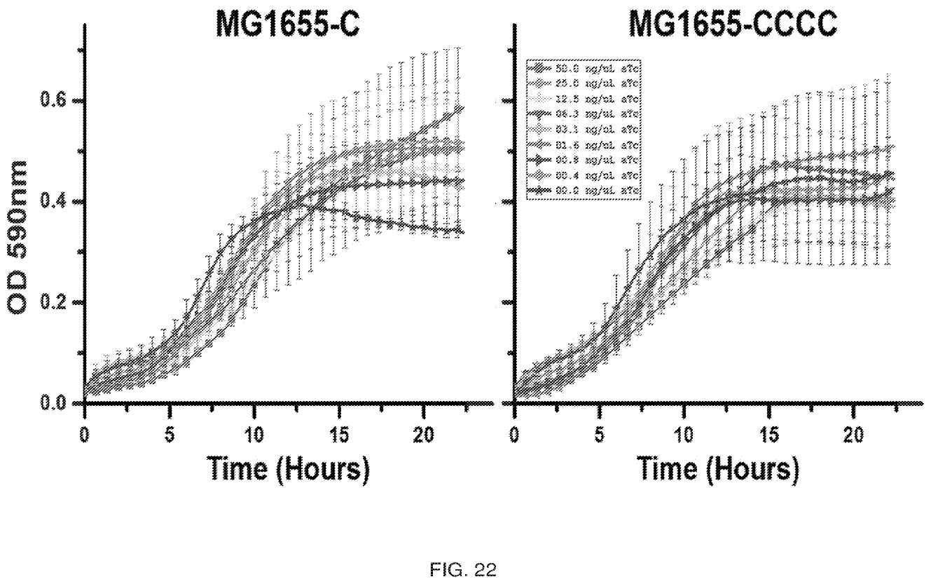

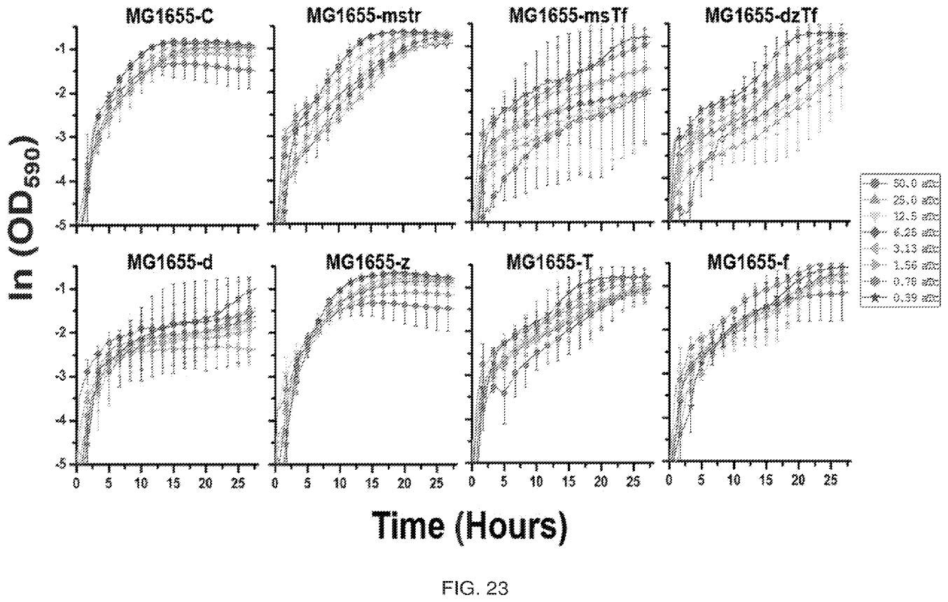

To test whether these fitness impacts arose in the absence of ciprofloxacin exposure, a subset of these strains were grown in the absence of ciprofloxacin at a range of aTc concentrations (FIGS. 22-23). FIG. 22 depicts growth curves of control strains under various concentrations of aTc, representing various levels of induction of dCas9 and the CRISPR perturbation system at large. A slight growth deficit was observed from aTc toxicity correlating with higher concentrations. Pervious work has quantified the relationship between aTc induction and dCas9 expression. Error bars represent sd of biological triplicates. FIG. 23 depicts growth curves of select strains under various levels of induction of the CRISPR perturbation system. Optical densities are converted to logarithmic form and normalized to the starting value to highlight the exponential phase of growth.

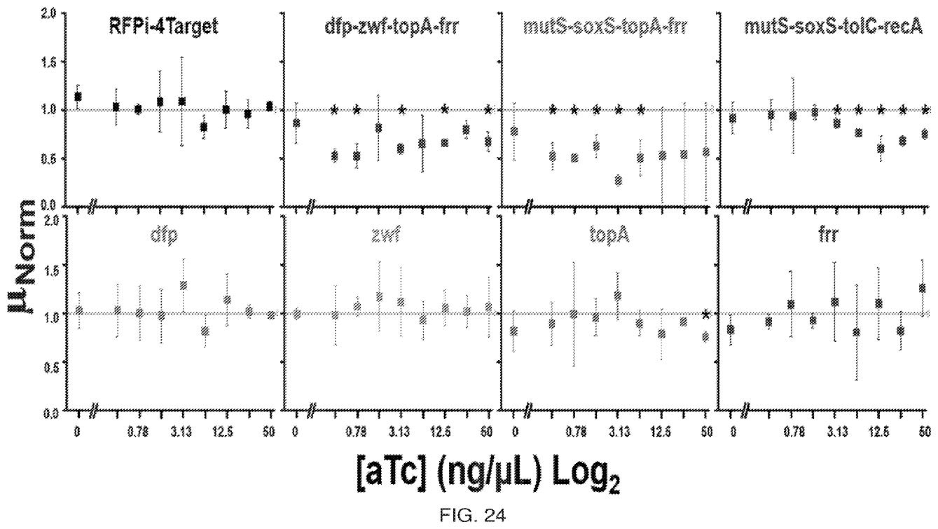

At higher aTc concentrations, a significant reduction of growth rates occurred in the four perturbation strains (FIG. 24). No significant changes in growth patterns of corresponding individual perturbations were observed. FIG. 24 depicts normalized growth rates of strains under different levels of induction of the CRISPR perturbation system. Growth rates are normalized to the RFPi-1 Target control under the same level of aTc. Astrisks indicate statistically significant differences in normalized growth rates form the RFPi-4 Target control under similar levels of aTc (P-value<0.05, two-tailed type II t-test).

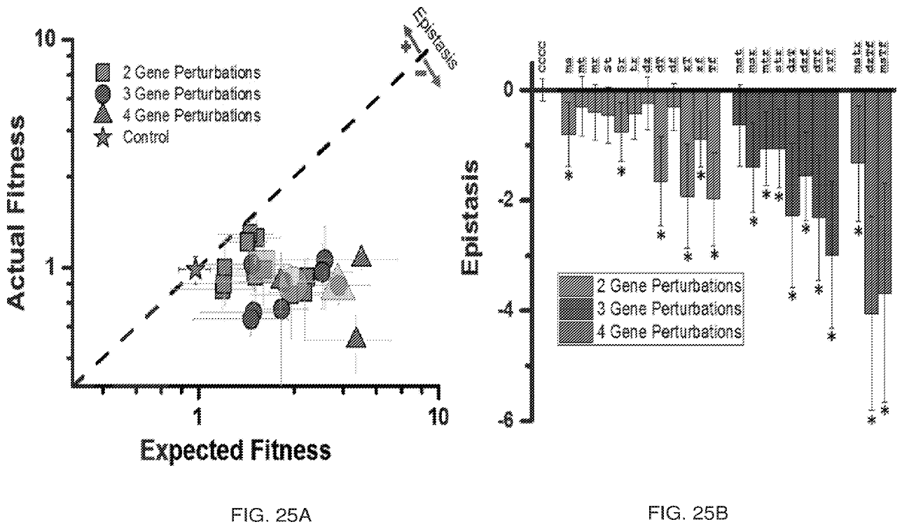

The epistatic interactions arising from two or more perturbations was then calculated (FIG. 25). FIG. 25 indicates the epistasis resulting from two or more gene perturbations. FIG. 25A indicates the relationship between expected and actual relative fitness. Centroid of each group (based on number of genes perturbed) is shown by larger bolded symbol. Dotted diagonal line indicates theoretical results if no epistasis was present. FIG. 25 B depicts the calculated epistasis of each strain. Error bars indicate s.d. (n=8). Asterisks indicate significant epistasis differences in relation to strain “CCCC” (P<0.01, two-tailed type II t-test).

In clustering the average values of expected versus actual fitness of each two, three, and four gene perturbation, a linear negative trends (r=−0.82) was observed, with no significant deviation between expected and actual fitness of the four gene perturbation control. These trends correlated into significant negative epistasis in half of two perturbation strains and all but one of three and four gene perturbation strains. The only gene pairs known to interact (mutS-recA and soxS-tolC) did not demonstrate significant epistasis, indicating that direct interaction is not required to produce negative epistatic effects. The degree of negative epistasis also increased between two and three perturbations (P=0.02) and three and four perturbations (P=0.07). Perturbations within set II demonstrated statistically greater levels of epistasis than those in set I (P=0.08 and P=0.01 for two and three perturbations respectively).

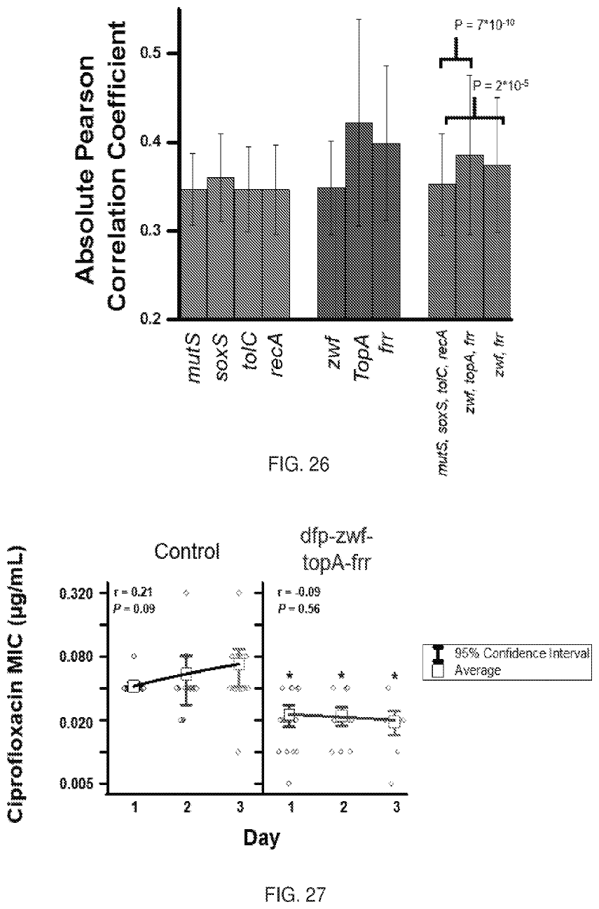

Analysis of available genome-wide interaction screens indicate that mutations of genes within set II induce stronger phenotypic interactions than mutations within set I (FIG. 26; P=7*10⁻¹⁰). The data presented here indicate that simultaneous perturbations produce detrimental fitness impacts that underlies gene expression trends and thus adaptation at large. FIG. 26 depicts the average absolute Pearson Correlation Coefficient (PCC) of all sets of statistically significant genetic interactions determined from a previous study. In this previous study, approximately 600,000 double-mutant strains were created from 163 gene knockouts crossed with 3,968 non-essential single gene deletions and 149 hypomorphic mutations. PCCs of phenotypes were calculated for every gene against all other genes, depicting the relative strength of that gene's genetic interaction with other genes. The average of the absolute value of each reported PCC of both individual genes was taken, as well as the average within each set of genes investigated. This study did not investigate dfp as it is an essential gene and not available as a knockout. topA is also an essential gene, but was included as a hypomorphic mutant, hence why a second calculation was included of just zwf and frr. Using both comparisons, we note that the average value of the PCC of the mutS-soxS-tolC-recA set is less than that of the dfp-zwf-topA-frr set, indicating that the genetic interactions are weaker.

To test whether such “emergent” negative epigenetic epistasis can artificially control adaptation rates, a subset of strains were exposed to ciprofloxacin over three days of exposure and changes in minimum inhibitory concentration (MIC) were quantified (FIG. 23) to serve as a proxy for how well each strain might adapt to clinical antibiotic treatment. A short term of adaptation was investigated to limit the possibility of inactivation of the CRISPR perturbation constructs through mutations. At the end of one day of exposure to ciprofloxacin, each four perturbation strain as well as the individual perturbation of topA exhibited statistically lower MICs than the control (P=5*10⁻¹⁵ 4*10⁻⁷, 5*10⁻⁴, 5*10⁻³ for T, dzTf, mstr and msTf respectively). The notable discrepancy between fitness and MIC impacts of strain T is likely due to the aforementioned dependency of topA gene perturbation on cell phase. At low ciprofloxacin exposure, strain T still expresses enough TopA to transition to log phase, at which point expression increases and a competitive advantage is afforded. At high ciprofloxacin concentrations, the cells never reach log phase and therefore are unable to proliferate.

After another day of exposure, the MIC of each strain increased with the exception of dzTf (0.022±0.10, P=0.0003) and msTf (0.022±0.17, P=0.0005). These strains also failed to achieve control MIC levels on day 3 (0.019±0.11, P=0.0004 and 0.031±0.17, P=0.0002 for dzTf and msTf respectively). One replicate of the control strain was found to grow at the highest concentration of ciprofloxacin (0.32 ng/mL) and was removed from data analysis using Grubb's test for outliers. A significant number of dzTf replicates failed to grow even under no ciprofloxacin exposure—three, six, and 15 replicates after one, two, and three days respectively. None of the other strains demonstrated this phenomenon, and the results were reproducible in a separate experiment (FIG. 24). The data presented in FIG. 27 represents only replicates which succeeded to grow in the absence ciprofloxacin, meaning that even if the strain did survive, its MIC failed to improve significantly (FIG. 26). Linear fits of MICs over each day represent the adaptive trajectory of each strain. The control strain, as well as strains z and T, demonstrated significant trends towards increasing MICs. Strains d, f and mstr demonstrated less significant trends of increasing MICs. Neither strain msTf nor dzTf exhibited positive correlations, indicating that these strains were less prone to adaptation than the control.

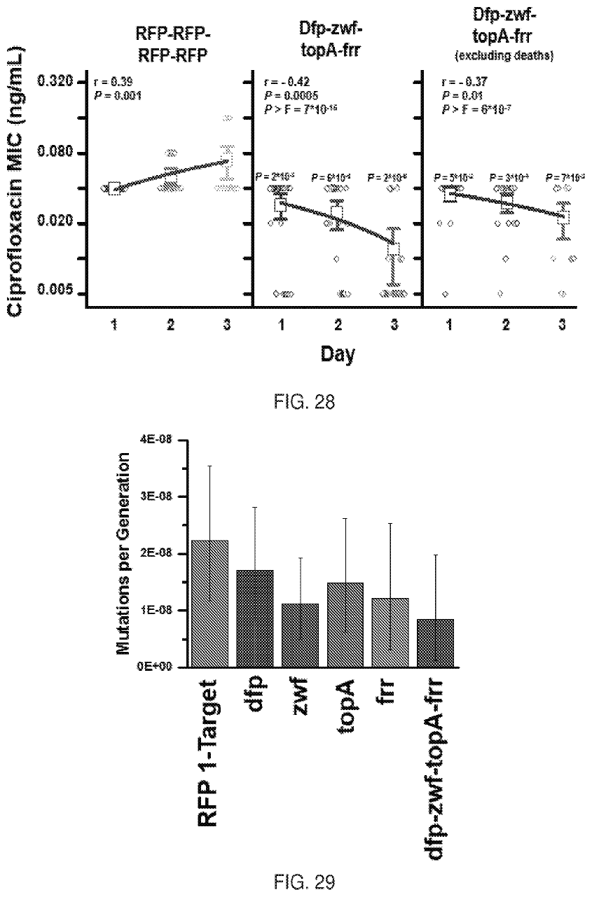

FIG. 28 indicates average MICs of strain dzTf compared to the control in a separate experiment to confirm the results of FIG. 27. Replicates of dzTf that failed to grow under 0 ciprofloxacin exposure were treated as having a MIC of 0.005 ng/mL (the lowest concentration tested) in the middle plot, or were excluded from analysis in the right plot. This affected five, five, and 13 replicates on days one, two, and three of the experiment respectively. Statistical significance of Pearson Correlation Coefficients are listed underneath their corresponding fits. The probability that the Pearson Correlation Coefficient is statistically different than the control is listed as P>F on the two graphs on the right. P values above each data average are in relation to the control from the same day of the experiment, using a two-tailed type II t-test.

To determine if these differences in MICs over time translated to the genetic level, two technical replicates of three biological replicates from each strain were sequenced at the end of three days of ciprofloxacin exposure. Mutations in gyrA were chosen, as S83L and D87Y have been demonstrated to confer resistance and is likely to be the first genetic change evolving E. coli will experience. Sequencing of gyrA revealed only two replicates with mutations; D87Y in one Control replicate with a MIC of 0.32 μg/mL and deletion of codon S83 in one T replicate with a MIC of 0.08 μg/mL. A mutation fluctuation assay also revealed similar mutation rates across a subset of strains, with the exception of dfp perturbation whose mutation rate was roughly doubled (FIG. 29).

FIG. 29 depicts mutation rates of strains as determined by the mutation fluctuation assay outlined by Luria and Delbruck. Values indicate the number of mutations per generation that arise during one day of CRIPSR perturbation. All comparisons were made in relation to the control strain from the same experimental run. Error bars indicate 95% confidence interval.

The data presented here demonstrates that epigenetic epistasis applies even to genes without direct genetic interactions (FIG. 25). The results demonstrate that one can engineer control over adaptation.

### Example 9—Materials and Methods for Example 8

### Plasmid and Strain Construction.

A list of plasmids and strains used in this study can be found in Tables 11 and 12 respectively. A two-plasmid system was utilized to induce native gene expression perturbation; the first plasmid encoded a sgRNA target sequence, while the second encoded either dCas9 or dCas9-w for gene inhibition and activation respectively. Addgene plasmid 44251 was used directly for targeting rfp inhibition (the “Control”—C), and also served as the starting plasmid for creating all subsequent sgRNA plasmids. Addgene plasmid 44251 was used directly for providing dCas9, while the previously constructed pPO-dCas9ω plasmid was used directly for providing dCas9-w. New sgRNA target plasmids were created by replacing the RFP-targeting sequence in 44251 with new gene sequences specific to the target of interest. This was accomplished by designing unique forward primers (listed in table 13) flanked with an ApaI restriction site and encoding the new target sequence. A common reverse primer flanked with XhoI was used alongside these primers to perform PCR amplification with Phusion High-Fidelity DNA Polymerase (New England Biolabs) of DNA inserts, which were subsequently digested with Cutsmart XhoI and ApaI (New England Biolabs) alongside 44251 backbone. Ligation of these pieces was performed using T4 DNA Ligase (Thermo Scientific), which were subsequently transformed into electrocompetent NEB 10-β. Transformants were minipreped using Zyppy Plasmid Miniprep Kit (Zymo Research Corporation) and submitted for sequencing to confirm successful insertion (GENEWIZ). As depicted by FIG. 17, sgRNA plasmids targeting individual genes were used to construct sgRNA plasmids targeting two or more genes via Gibson Assembly, for which a common forward and reverse primer was used to amplify the first sgRNA target plasmid while introducing overhangs downstream of the terminator sequence following the first target. A common set of primers were then utilized to amplify sgRNA targets to the second, third, and fourth targets depending on the intended number of final sgRNA targets. A batch Gibson reaction was performed at 50° C. for 3 h with T5 exonuclease (New England Biolabs), Phusion polymerase and Taq ligase (New England Biolabs) on this one backbone and one to three inserts to stitch all pieces together. sgRNA-C-mCherry was constructed by amplifying constitutively expressed mCherry from pFPV-mCherry (Addgene 20956) and inserting into sgRNA-C upstream of the sgRNA sequence. Final experimental sgRNA plasmids were transformed into chemically competent E. coli strain K-12 MG1655 (ATCC 700926) harboring either 44249 or pPO-dCas9ω if the target was meant to inhibit or activate expression respectively. This process was used to construct all 33 control and experimental strains used in the study.

TABLE 11
Strains used in this study. The host strain is MG1655 for all strains.

| Strain | Cas9 Phenotype | Guide-RNA |
|---|---|---|
| C | dCas9 | rfpi |
| CCCC | dCas9 | rfpi-rfpi-rfpi-rfpi |
| C-mCherry | dCas9 | rfpi + mCherry |
| m | dCas9 | mutSa |
| s | dCas9 | soxSa |
| t | dCas9 | tolCa |
| r | dCas9 | recAa |
| ms | dCas9 | mutSa-soxSa |
| mt | dCas9 | mutSa-tolCa |
| mr | dCas9 | mutSa-recAa |
| st | dCas9 | soxSa-tolCa |
| sr | dCas9 | soxSa-recAa |
| tr | dCas9 | tolCa-recAa |
| mst | dCas9 | mutSa-soxSa-tolCa |
| msr | dCas9 | mutSa-soxSa-recAa |
| mtr | dCas9 | mutSa-tolCa-recAa |
| str | dCas9 | soxSa-tolCa-recAa |
| mstr | dCas9 | mutSa-soxSa-tolCa-recAa |
| d | dCas9-ω | dfpi |
| z | dCas9-ω | zwfi |
| T | dCas9-ω | topAi |
| f | dCas9-ω | frri |
| dz | dCas9-ω | dfpi-zwfi |
| dT | dCas9-ω | dfpi-topAi |
| df | dCas9-ω | dfpi-frri |
| zT | dCas9-ω | zwfi-topAi |
| zf | dCas9-ω | zwfi-frri |
| Tf | dCas9-ω | topAi-frri |
| dzT | dCas9-ω | dfpi-zwfi-topAi |
| dzf | dCas9-ω | dfpi-zwfi-frri |
| dTf | dCas9-ω | dfpi-topAi-frri |
| zTf | dCas9-ω | zwfi-topAi-frri |
| dzTf | dCas9-ω | dfpi-zwfi-topAi-frri |
| msTf | dCas9-ω | mutSa-soxSa-topAi-frri |

TABLE 12
Raw epistasis calculations.

| | Expected Fitness | | Epistasis | | |
|---|---|---|---|---|---|
| Strain | Avg | StDev | Avg | StDev | P |
| CCCC | 0.97 | 0.15 | +0.01 | 0.20 | 0.912 |
| ms | 1.72 | 0.52 | −0.81 | 0.57 | 0.005 |
| mt | 1.29 | 0.46 | −0.29 | 0.55 | 0.174 |
| mr | 1.75 | 0.44 | −0.40 | 0.51 | 0.063 |
| st | 1.26 | 0.49 | −0.46 | 0.51 | 0.039 |
| sr | 1.71 | 0.51 | −0.76 | 0.54 | 0.005 |
| tr | 1.28 | 0.45 | −0.43 | 0.46 | 0.035 |
| mst | 1.67 | 0.72 | −0.64 | 0.75 | 0.047 |
| msr | 2.27 | 0.80 | −1.40 | 0.81 | 0.002 |
| mtr | 1.69 | 0.67 | −1.06 | 0.68 | 0.003 |
| str | 1.65 | 0.71 | −1.06 | 0.71 | 0.004 |
| mstr | 2.20 | 1.02 | −1.33 | 1.05 | 0.009 |
| dz | 1.64 | 0.43 | −0.24 | 0.48 | 0.201 |
| dT | 2.42 | 0.76 | −1.66 | 0.81 | 0.001 |
| df | 1.60 | 0.33 | −0.30 | 0.43 | 0.085 |
| zT | 2.84 | 0.93 | −1.93 | 0.95 | 0.001 |
| zf | 1.87 | 0.42 | −0.89 | 0.52 | 0.002 |
| Tf | 2.76 | 0.79 | −1.98 | 0.85 | 3E−4 |
| dzT | 3.36 | 1.24 | −2.28 | 1.31 | 0.002 |
| dzf | 2.22 | 0.63 | −1.56 | 0.80 | 0.001 |
| dTf | 3.27 | 1.10 | −2.31 | 1.14 | 0.001 |
| zTf | 3.83 | 1.32 | −2.99 | 1.33 | 4E−4 |
| dzTf | 4.53 | 1.75 | −4.06 | 1.76 | 3E−4 |
| msTf | 4.76 | 1.98 | −3.68 | 1.99 | 0.001 |

TABLE 13
Raw MIC values of Ciprofloxacin (ng/mL) at the end of each day (D).

| | d | | | z | | | T | | | f | | |
|---|---|---|---|---|---|---|---|---|---|---|---|---|
| Replicate | D 1 | D 2 | D 3 | D 1 | D 2 | D 3 | D 1 | D 2 | D 3 | D 1 | D 2 | D 3 |
| 1 | 0.04 | 0.04 | 0.04 | 0.04 | 0.08 | 0.08 | 0.02 | 0.04 | 0.04 | 0.04 | 0.04 | 0.08 |
| 2 | 0.02 | 0.04 | 0.08 | 0.04 | 0.04 | 0.04 | 0.02 | 0.02 | 0.04 | 0.04 | 0.01 | 0.04 |
| 3 | 0.04 | 0.04 | 0.04 | 0.04 | 0.08 | 0.08 | 0.01 | 0.02 | 0.04 | 0.04 | 0.02 | 0.04 |
| 4 | 0.04 | 0.04 | 0.08 | 0.04 | 0.04 | 0.04 | 0.02 | 0.02 | 0.04 | 0.04 | 0.08 | 0.08 |
| 5 | 0.04 | 0.08 | 0.08 | 0.04 | 0.04 | 0.08 | 0.02 | 0.04 | 0.08 | 0.04 | 0.04 | 0.04 |
| 6 | 0.04 | 0.04 | 0.04 | 0.04 | 0.08 | 0.08 | 0.02 | 0.02 | 0.02 | 0.04 | 0.04 | 0.04 |
| 7 | 0.04 | 0.04 | 0.04 | 0.04 | 0.04 | 0.08 | 0.02 | 0.04 | 0.04 | 0.04 | 0.04 | 0.08 |
| 8 | 0.04 | 0.04 | 0.04 | 0.04 | 0.04 | 0.04 | 0.02 | 0.04 | 0.02 | 0.04 | 0.01 | 0.04 |
| 9 | 0.02 | 0.04 | 0.04 | 0.04 | 0.04 | 0.04 | 0.02 | 0.04 | 0.04 | 0.04 | 0.04 | 0.04 |
| 10 | 0.04 | 0.04 | 0.04 | 0.04 | 0.04 | 0.04 | 0.02 | 0.04 | 0.02 | 0.04 | 0.04 | 0.08 |
| 11 | 0.02 | 0.02 | 0.04 | 0.04 | 0.04 | 0.16 | 0.02 | 0.02 | 0.02 | 0.04 | 0.04 | 0.04 |
| 12 | 0.04 | 0.04 | 0.04 | 0.04 | 0.08 | 0.08 | 0.02 | 0.02 | 0.02 | 0.04 | 0.04 | 0.04 |
| 13 | 0.04 | 0.04 | 0.04 | 0.04 | 0.04 | 0.04 | 0.02 | 0.02 | 0.04 | 0.04 | 0.01 | 0.01 |
| 14 | 0.04 | 0.04 | 0.01 | 0.04 | 0.04 | 0.08 | 0.02 | 0.02 | 0.08 | 0.04 | 0.04 | 0.04 |
| 15 | 0.04 | 0.08 | 0.08 | 0.04 | 0.04 | 0.04 | 0.02 | 0.04 | 0.08 | 0.04 | 0.04 | 0.08 |
| 16 | 0.04 | 0.04 | 0.08 | 0.04 | 0.04 | 0.04 | 0.02 | 0.04 | 0.08 | 0.04 | 0.04 | 0.04 |
| 17 | 0.02 | 0.04 | 0.04 | 0.04 | 0.02 | 0.04 | 0.02 | 0.04 | 0.04 | 0.04 | 0.04 | 0.04 |
| 18 | 0.02 | 0.04 | 0.08 | 0.04 | 0.04 | 0.04 | 0.02 | 0.04 | 0.08 | 0.04 | 0.04 | 0.04 |
| 19 | 0.04 | 0.04 | 0.04 | 0.02 | 0.04 | 0.08 | 0.02 | 0.02 | 0.02 | 0.04 | 0.04 | 0.04 |
| 20 | 0.04 | 0.04 | 0.04 | 0.04 | 0.04 | 0.04 | 0.02 | 0.04 | 0.04 | 0.04 | 0.16 | 0.16 |
| 21 | 0.04 | 0.04 | 0.04 | 0.04 | 0.04 | 0.04 | 0.02 | 0.02 | 0.04 | 0.04 | 0.04 | 0.04 |
| 22 | 0.04 | 0.04 | 0.01 | 0.04 | 0.04 | 0.08 | 0.02 | 0.04 | 0.04 | 0.02 | 0.01 | 0.04 |
| | C | | | mstr | | | msTf | | | dzTf | | |
| Replicate | D 1 | D 2 | D 3 | D 1 | D 2 | D 3 | D 1 | D 2 | D 3 | D 1 | D 2 | D 3 |
| 1 | 0.04 | 0.08 | 0.08 | 0.04 | 0.01 | 0.04 | 0.04 | 0.01 | 0.02 | .005 | 0.02 | 0.02 |
| 2 | 0.04 | 0.04 | 0.04 | 0.02 | 0.04 | 0.04 | 0.02 | 0.02 | 0.02 | 0.02 | 0.01 | .005 |
| 3 | 0.08 | 0.32 | 0.32 | 0.02 | 0.02 | 0.02 | 0.04 | 0.04 | 0.04 | 0.02 | 0.02 | |
| 4 | 0.04 | 0.04 | 0.08 | 0.02 | 0.04 | 0.04 | 0.02 | 0.02 | 0.04 | | | |
| 5 | 0.04 | 0.04 | 0.01 | 0.02 | 0.02 | 0.02 | 0.08 | 0.01 | 0.04 | 0.01 | 0.02 | |
| 6 | 0.04 | 0.04 | 0.08 | 0.02 | 0.02 | 0.02 | 0.04 | 0.01 | 0.04 | 0.04 | 0.02 | |
| 7 | 0.04 | 0.08 | 0.08 | 0.04 | 0.01 | 0.04 | 0.04 | 0.02 | 0.02 | 0.02 | 0.02 | |
| 8 | 0.04 | 0.04 | 0.04 | 0.02 | 0.02 | 0.02 | 0.02 | 0.04 | 0.04 | 0.04 | 0.04 | 0.01 |
| 9 | 0.04 | 0.02 | 0.04 | 0.08 | 0.16 | 0.16 | 0.04 | 0.01 | 0.04 | 0.02 | | |
| 10 | 0.04 | 0.04 | 0.08 | 0.02 | 0.02 | 0.04 | 0.02 | 0.02 | 0.02 | 0.01 | 0.02 | |
| 11 | 0.04 | 0.02 | 0.04 | 0.02 | 0.04 | 0.04 | 0.04 | 0.01 | 0.01 | | | |
| 12 | 0.04 | 0.04 | 0.04 | 0.02 | 0.02 | 0.04 | 0.02 | 0.01 | 0.04 | 0.02 | 0.04 | 0.04 |
| 13 | 0.04 | 0.02 | 0.04 | 0.02 | 0.04 | 0.02 | 0.04 | 0.08 | 0.01 | 0.02 | 0.02 | |
| 14 | 0.04 | 0.04 | 0.04 | 0.04 | 0.04 | 0.04 | 0.04 | 0.01 | 0.02 | 0.04 | | |
| 15 | 0.04 | 0.02 | 0.08 | 0.02 | 0.02 | 0.02 | 0.02 | 0.04 | 0.04 | 0.02 | 0.02 | 0.02 |
| 16 | 0.04 | 0.04 | 0.04 | 0.02 | 0.04 | 0.04 | 0.04 | 0.01 | 0.04 | 0.02 | 0.02 | |
| 17 | 0.04 | 0.04 | 0.08 | 0.02 | 0.04 | 0.02 | 0.02 | 0.01 | 0.04 | | | |
| 18 | 0.04 | 0.04 | 0.08 | 0.04 | 0.04 | 0.08 | 0.02 | 0.02 | 0.01 | 0.01 | 0.01 | |
| 19 | 0.04 | 0.04 | 0.04 | 0.04 | 0.08 | 0.04 | 0.02 | 0.02 | 0.01 | 0.02 | 0.04 | |
| 20 | 0.04 | 0.08 | 0.08 | 0.04 | 0.04 | 0.04 | 0.02 | 0.01 | 0.04 | 0.04 | 0.02 | 0.02 |
| 21 | 0.04 | 0.04 | 0.04 | 0.02 | 0.04 | 0.04 | 0.02 | 0.02 | 0.02 | 0.01 | | |
| 22 | 0.04 | 0.04 | 0.04 | 0.02 | 0.02 | 0.02 | 0.02 | 0.04 | 0.08 | 0.04 | 0.01 | 0.02 |

### Media and Culture Conditions.

All cultures were grown in Luria-Bertani Broth (LB) (Sigma-Aldrich), with the exception of RT-qPCR samples and samples for growth/lag time calculations which were grown in M9 minimal media (5×M9 minimal media salts solution from MP Biomedicals, 2.0 mM MgSO₄, and 0.1 mM CaCl₂ supplemented with 0.4% weight/vol glucose). Plates and media were supplemented with ampicillin (100 μg/mL) or chloramphenicol (35 μg/mL) to maintain selection of sgRNA plasmids or dCas9/dCas9-ω plasmids respectively. aTc was used to induce CRISPR expression at a final concentration of 10 ng/mL, except where otherwise noted. The authors also note that the aTc-inducible promoter driving expression of dCas9 is not P_{L}tetO-1 as originally reported (Qi), but rather a tet-promoter variant with only one Tet binding site highly similar to the original tet-promoter, indicating that slightly higher leaky expression is expected of dCas9 and dCas9-ω. All cultures were grown at 37° C., with shaking at 225 rpm unless otherwise noted. Cultures for competition were grown in 200 μL cultures in 96 well conical bottom microplates. Cultures for RT-qPCR were grown in 3 mL cultures. Cultures for CFU and MIC screens were grown in 100 μL cultures in 384 well microplates. Cultures for lag time and growth rate calculations were grown in 100 μL cultures in 384 well microplates in a GENios plate reader (Tecan Group Ltd.) operating under Magellan software (version 7.2) with 16.6 min of shaking before measurement of optical densities at 590 nm absorbance every 20 min.

### Quantitative Reverse Transcription PCR.

The degree of gene expression perturbation was confirmed by subjecting biological triplicates of each individual gene perturbation to RT-qPCR, as well as constructs perturbing four genes simultaneously. Cultures were inoculated from individual colonies and grown for 20 hours overnight in 3 mL M9 cultures and subsequently diluted 1:100 the following morning into 3 mL of fresh media containing aTc. These cultures were grown for 8 hours before RNA extraction using the GeneJet RNA Purification Kit (Thermo Scientific) and purification using Turbo DNA-free kit (Ambion). Purified RNA was used to create cDNA using the Maxima First Strand cDNA Synthesis Kit for RT-qPCR (Thermo Scientific). Technical duplicates of each replicate was subjected to RT-qPCR reactions from the Maxima SYBR Green qPCR Master Mix (Thermo Scientific) using 2 ng of cDNA in 20 μL reactions run on a QuantStudio 6 Flex Real-Time PCR System (Applied Biosystems) in the CU Core Sequencing Facility, in which reactions were allowed to run for 40 cycles with Rox normalization. Gene expression changes were calculated using ^{2-ΔΔCq} values calculated from averages of technical duplicates.

### Competition Fitness Assay.

Fitness of each perturbed strain was calculated by competing said strain against the red fluorescent dCas9-C-mCherry control strain. A total of eight biological replicates were inoculated from colonies into 200 μL in 96 well plates and grown overnight for 16 h with selection. Cultures were then diluted 1:100 into 200 μL of media with selection and 10 ng/μL aTc to induce gene perturbation, and grown for another 24 hours. Competition was initiated by diluting cultures 1:100 and mixing equal cell ratios of the red control strain with each experimental strain into 200 μL of media containing selection, aTc and 0.005 ng/μL ciprofloxacin. To determine starting ratios of each strain, two μL was used for plating of 50 μL of 1:10000 and 1:100000 dilutions. The remaining culture was grown for another 24 h, diluted 1:100 into the same media, and grown again for 24 h. At the end of this growth period, 50 μL of 1:10000 and 1:100000 dilutions were again prepared and plated to determine the ending cell ratios. Two plate images were taken with fluorescence activation at 540 nm, one with emission filtering at 590 nm and the other without, and these images were overlaid to facilitate colony counting. Colony counts were used to determine fitness values (w) using the standard Malthusian Fitness Equation (cite), using the formula ω=ln(N_{E1}*100²/N_{E0})/ln(N_{C1}*100²/N_{C0}) where the variables are defined as follows: “N”—CFU, “E”—experimental strain, “C”—control strain, “1”—after exposure, and “0”—before exposure. Fitness values were calculated as such for all 30 experimental strains, as well as the control strains targeting RFP with one or four sgRNAs and expressing no fluorescence. Expected fitness values (ω_{E}) for strains with perturbation of two or more genes was calculated using assuming a multiplicative model as follows:

$${ω}_{E} = {\prod }_{i = 1}^{n} {ω}_{i}$$

where n expands to all sets of genes perturbed. For instance, ω_{E} of MG1655-dzf would be calculated as the product of fitness from each individual gene perturbation (ω_{d}*ω_{z}*ω_{f}). Epistasis was calculated as the difference between measured fitness (ω) and expected fitness (ω_{E}). We then determined whether epistasis values deviated from the null hypothesis (no epistasis) using standard error to determine the 95% confidence interval and subsequently performing a z-test (assuming two-tailed distribution) to obtain P-values.

### Growth Assay.

To demonstrate growth phenotypes including lag times and growth rates, biological triplicates of each strain were inoculated from individual colonies into 150 μL of LB containing selection in a conical 96 well microplate and grown for seven h. After initial growth, 1 μL of each culture was used to inoculate 2 mL of M9 media containing selection and grown for 16 h. The follow morning, each culture was diluted 1:100 into 100 μL M9 media cultures containing selection and a variable concentration of aTc from 0 to 50 ng/μL. These cultures were grown in a 384 well microplate in a Tecan Genios reader for 24 hours, measuring OD 590 nm every 20 min.

### Minimum Inhibitory Concentration Assays.

MIC assays were performed using 22 biological replicates per strain. Individual colonies were inoculated into 100 μL LB cultures containing selection and grown for 16 h overnight. The following morning, cultures were diluted 1:50 into 100 μL of fresh media containing 10 ng/μL aTc in 384 well plates and grown another 24 h. The following day, each replicate was diluted 1:50 into fresh media containing selection, aTc, and a range of ciprofloxacin concentrations including 0, 0.005, 0.01, 0.02, 0.04, 0.08, and 0.16 ng/μL ciprofloxacin to begin the MIC screen. The new 384 well plate containing variable ciprofloxacin concentrations was grown for 24 h (Day 0 to Day 1), after which absorbance were measured at 590 nm. Cultures expressing ODs greater than 0.15 were determined to have survived. The highest concentration at which each replicate survived was used to inoculate the same plate setup as defined previously (Day 0 to Day 1), while the next highest concentration was determined to be the MIC. This process was repeated for one more day to obtain MICs for 22 cultures at the end of each day of growth for all three days. Three replicates of each strain were saved as glycerol stocks for subsequent sequencing.

### Mutation Sequencing Assay.

Glycerol stocks of strains saved after three days of ciprofloxacin exposure were streaked onto LB agar plates with selection and grown overnight. Two colonies from each plate were used to perform colony PCR amplification of gyrA in the 1200 bp region surrounding S83 and D87, the most likely regions for mutations conferring ciprofloxacin resistance to arise. PCR samples were purified and submitted for sequencing (GENEWIZ), for a total of six samples per strain.

### Mutation Fluctuation Assay.

Mutation rates were estimated using the rifampicin exposure approach outlined by Luria and Delbruck. Individual colonies were grown in 1 mL LB without selection for 16 hours and subsequently adjusted to normalized ODs with addition of LB to denser cultures. Each culture was used to diluted 1:10,000 into 33 parallel 100 μL cultures of LB supplemented with aTc and selection and grown for 24 hours. Colony forming units were estimated from three replicates on plain LB agar paltes, while the remaining 30 cultures were plated on LB agar containing 100 μg/mL rifampicin. Colonies were counted after 48 hours of exposure, and the FALCOR web tool was used to estimate mutation rates.

All of the compositions and methods disclosed and claimed herein can be made and executed without undue experimentation in light of the present disclosure. While the compositions and methods have been described in terms of particular embodiments, it is apparent to those of skill in the art that variations maybe applied to the compositions and methods and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

1. A method for altering bacterial fitness of a bacterium, the method comprising modulating gene expression of at least 3 genes in the bacterium, wherein the at least 3 genes comprise bacterial stress response genes, bacterial essential genes, or a combination of bacterial stress response genes and bacterial essential genes, and wherein modulating the gene expression of the at least 3 genes comprises modulating gene expression by modulating gene transcription, post-transcription modulation of gene expression, or both modulating gene transcription and post-transcription modulation of gene expression.

2.-4. (canceled)

5. The method according to claim 1, wherein the bacterial stress response genes comprise mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and the bacterial essential genes comprise dfp, topA, zwf, rfp, and frr.

6. (canceled)

7. The method according to claim 1, wherein the at least 3 genes comprise genes selected from the group consisting of mutS, soxS, and tolC; mutS, soxS, and recA; mutS, tolC, and recA; soxS, tolC, and recA; dfp, zwf, and topA; dfp, zwf, and frr; dfp, topA, and frr; mutS, soxS, tolC, and recA; dfp, zwf, topA, and frr; and mutS, soxS, topA, and frr.

8. (canceled)

9. The method according to claim 1, wherein modulating the gene expression comprises modulating the gene expression at a transcriptional level by delivering to the bacterium at least one of a CRISPR/Cas system, a transcription activator-like effector (TALE) system, a zinc-finger protein system, a synthetic polyamide system, and a meganuclease system.

10. The method according to claim 1, wherein modulating the gene expression comprises modulating the gene expression at a post-transcriptional level by delivering to the bacterium at least one of a morpholino-based system, a peptide nucleic acid-based system, and a locked nucleic acid-based system.

11. The method according to claim 1, wherein modulating the gene expression of the at least 3 genes comprises delivering to the bacterium a CRISPR/Cas system comprising a catalytically dead CRISPR-associated (dCas) protein and at least three guide RNA (gRNA) molecules or one or more expression vectors encoding the dCas and the at least three gRNAs, wherein each of the at least three gRNA molecules comprise a CRISPR-associated (Cas) protein binding site and a targeting RNA sequence, wherein the targeting RNA sequence of each of the at least three gRNA molecules targets one gene of the at least 3 genes.

12.-14. (canceled)

15. The method according to claim 11, wherein the targeting RNA sequence targets a regulatory region of the targeted gene.

16. The method according to claim 15, wherein the CRISPR/Cas system further comprises a transcriptional effector molecule associated with the dCas protein, the transcriptional effector molecule being selected from the group consisting of a DNA methylase, a histone acetylase, and an RNA polymerase ω-subunit.

17.-20. (canceled)

21. The method according to claim 15, wherein the dCas protein is encoded by a first nucleic acid sequence and each of the at least three gRNA molecules is encoded by an additional nucleic acid sequence.

22. (canceled)

23. The method according to claim 21, wherein a single expression vector comprises the first nucleic acid sequence and the additional nucleic acid sequences, or two or more expression vectors each comprise one of, or a combination of, the first nucleic acid sequence and one or more of the additional nucleic acid sequences.

24. (canceled)

25. (canceled)

26. The method according to claim 1, wherein modulating the gene expression of the at least 3 genes comprises delivering at least three peptide nucleic acids to the bacterium, wherein each of the at least three peptide nucleic acids comprises a sequence of 5 to 20 nucleic acids capable of hybridizing to a target sequence of one of the at least three genes.

27. (canceled)

28. (canceled)

29. The method according to claim 1, wherein the method is carried out in vivo or in vitro.

30. (canceled)

31. A CRISPR/Cas system for altering bacterial fitness of a bacterium comprising:
a catalytically-dead CRISPR-associated (dCas) protein; and
at least three guide RNA (gRNA) molecules, wherein each of the at least three gRNA molecules comprise a CRISPR-associated (Cas) protein binding site and a targeting RNA sequence specific for a different gene of the bacterium, and wherein the at least 3 genes comprise bacterial stress response genes, bacterial essential genes, or a combination of bacterial stress response genes and bacterial essential genes.

32. (canceled)

33. The CRISPR/Cas system of claim 31, wherein the stress response genes comprise mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, and mreD, and the bacterial essential genes comprise dfp, topA, zwf, rfp, and frr.

34. (canceled)

35. The CRISPR/Cas system of claim 31, wherein the at least three gRNA molecules target genes selected from the group consisting of mutS, soxS, and tolC; mutS, soxS, and recA; mutS, tolC, and recA; soxS, tolC, and recA; dfp, zwf, and topA; dfp, zwf, and frr; dfp, topA, and frr; mutS, soxS, tolC, and recA; dfp, zwf, topA, and frr; and mutS, soxS, topA, and frr.

36. The CRISPR/Cas system of claim 31, further comprising a transcriptional effector molecule associated with the dCas protein the transcriptional effector molecule being selected from the group consisting of a DNA methylase, a histone acetylase, and an RNA polymerase ω-subunit.

37. (canceled)

38. The CRISPR/Cas system of claim 31, wherein the CRISPR/Cas system is encapsulated in one or more nanoparticles, optionally wherein a surface of the one or more nanoparticles comprises at least one cell-specific targeting ligand for the bacterium selected from the group of an antibody, a cell-penetrating peptide, and a combination thereof.

39. (canceled)

40. A CRISPR/Cas system for altering bacterial fitness of a bacterium, comprising at least one expression vector, the at least one expression vector comprising:
a first nucleic acid sequence encoding a catalytically-dead CRISPR-associated (dCas) protein; and
at least three additional nucleic acid sequences, wherein each of the at least three additional nucleic acid sequences encodes a unique guide RNA (gRNA) molecule, wherein each unique gRNA molecule comprise a CRISPR-associated (Cas) protein binding site and a targeting RNA sequence specific for a unique gene of the bacterium wherein the at least three additional nucleic acid sequences each encode a unique gRNA that targets a stress response gene or a bacterial essential gene.

41. (canceled)

42. The CRISPR/Cas system of claim 40, wherein the stress response gene comprises mutS, soxS, tolC, acrA, recA, dinB, marA, folC, cdsA, msbA, lptA, sgrT, secA, secD, secE, secF, secM, secY, adk, coaD, eno, ispA, ispB, ispD, ispE, ispF, ispG, ispH, ispU, can, grpE, lexA, rseP, rpoE, ffh, ffs, lepB, lspA, odgE, ftsA, ftsB, ftsE, ftsI, ftsK, ftsL, ftsQ, ftsW, ftsZ, holA, holB, bamA, bamD, gyrA, gyrB, prfA, rpsA, rpsB, rpsC, rpsD, rpsE, rpsH, rpsJ, rpsK, rpsL, rpsN, rpsP, rpsR, rpsS, ligA, prmC, trmD, fnrS, ilvX, apbE, nusA, rpoD, nusE, ffh, rpsU, accD, degS, ftsN, lolA, hflB, mraY, rsG, rplV, nadD, murF, murA, or mreD, and the bacterial essential genes comprises dfp, topA, zwf, rfp, and frr.

43. (canceled)

44. The CRISPR/Cas system of claim 40, wherein the at least three additional nucleic acid sequences encode a group of unique gRNAs that target genes selected from the group consisting of mutS, soxS, and tolC; mutS, soxS, and recA; mutS, tolC, and recA; soxS, tolC, and recA; dfp, zwf, and topA; dfp, zwf, and frr; dfp, topA, and frr; mutS, soxS, tolC, and recA; dfp, zwf, topA, and frr; and mutS, soxS, topA, and frr.

45. The CRISPR/Cas system of claim 40, wherein the first nucleic acid encodes a dCas fusion protein, wherein dCas is fused to a transcriptional effector molecule, the transcriptional effector molecule selected from the group consisting of a DNA methylase, a histone acetylase, and an RNA polymerase ω-subunit.

46. (canceled)

47. The CRISPR/Cas system of claim 40, wherein a single expression vector comprises the first nucleic acid sequence and the at least three additional nucleic acid sequences, or wherein each of two or more expression vectors comprise one of, or a combination of, the first nucleic acid sequence and one or more of the at least three additional nucleic acid sequences.

48. (canceled)

49. The CRISPR/Cas system of claim 40, wherein the at least one expression vector is encapsulated in one or more nanoparticles, incorporated into a bacteriophage, or incorporated into a donor cell.

50.-62. (canceled)
